SEQUENCE LISTING (18 sequences)

SEQ ID NO: 1 - protein, 8 aa, Artificial Sequence
  Synthetic Construct
GFTFSNYA

SEQ ID NO: 2 - protein, 8 aa, Artificial Sequence
  Synthetic Construct
ISGSGDYT

SEQ ID NO: 3 - protein, 11 aa, Artificial Sequence
  Synthetic Construct
ARSPWGYYLDS

SEQ ID NO: 4 - protein, 6 aa, Artificial Sequence
  Synthetic Construct
QGISSR

SEQ ID NO: 5 - protein, 3 aa, Artificial Sequence
  Synthetic Construct
AAS

SEQ ID NO: 6 - protein, 9 aa, Artificial Sequence
  Synthetic Construct
QQYNSYPYT

SEQ ID NO: 7 - protein, 118 aa, Artificial Sequence
  Synthetic Construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 8 - protein, 107 aa, Artificial Sequence
  Synthetic Construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 9 - protein, 25 aa, Artificial Sequence
  Synthetic Construct
EVQLLESGGGLVQPGGSLRLSCAAS

SEQ ID NO: 10 - protein, 17 aa, Artificial Sequence
  Synthetic Construct
MSWVRQAPGKGLEWVSS

SEQ ID NO: 11 - protein, 38 aa, Artificial Sequence
  Synthetic Construct
YYTDSVKGRFTISRDNSKNTLYLQMNSLRAEDTAVYYC

SEQ ID NO: 12 - protein, 11 aa, Artificial Sequence
  Synthetic Construct
WGQGTLVTVSS

SEQ ID NO: 13 - protein, 26 aa, Artificial Sequence
  Synthetic Construct
DIQMTQSPPSLSASAGDRVTITCRAS

SEQ ID NO: 14 - protein, 17 aa, Artificial Sequence
  Synthetic Construct
LAWYQQKPEKAPKSLIY

SEQ ID NO: 15 - protein, 36 aa, Artificial Sequence
  Synthetic Construct
SLQSGVPSRFSGSGSGTDFTLTISSLQPEDFATYYC

SEQ ID NO: 16 - protein, 10 aa, Artificial Sequence
  Synthetic Construct
FGQGTKLEIK

SEQ ID NO: 17 - protein, 448 aa, Artificial Sequence
  Synthetic Construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, Artificial Sequence
  Synthetic Construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0263902 A1
(43) Pub. Date: Aug. 24, 2023

(54) ANTI-TISSUE FACTOR ANTIBODY-DRUG CONJUGATES AND THEIR USE IN THE TREATMENT OF CANCER

(71) Applicant: Genmab A/S, OPENHAGEN V (DK)

(72) Inventor: Reshma A. RANGWALA, Philadelphia, PA (US)

(21) Appl. No.: 18/010,783

(22) PCT Filed: Jun. 29, 2021
(86) PCT No.: PCT/EP2021/067855

§ 371 Date: Dec. 15, 2022

Related U.S. Application Data
(60) Provisional application No. 63/094,571, filed on Oct. 21, 2020.
(60) Provisional application No. 63/045,448, filed on Jun. 29, 2020.

(51) Int. Cl. A61K 47/68; A61P 35/00; A61K 9/00

(52) U.S. Cl. CPC A61K 47/6803; A61K 47/6849; A61P 35/00; A61K 9/0019

(57) ABSTRACT

The invention provides methods and compositions for treating cancer, such as advanced cervical cancer, in a subject, such as by the administration of antibody-drug conjugates that bind to tissue factor (TF). The invention also provides articles of manufacture and compositions comprising said antibody drug-conjugates that bind to TF for use in treating cancer (e.g., advanced cervical cancer).

### FIELD OF THE INVENTION

The present invention relates to anti-tissue factor (TF) antibody-drug conjugates and methods of using the same to treat cancer, such as advanced cervical cancer.

### SUBMISSION OF SEQUENCE LISTING ON ASCII TEXT FILE

The content of the following submission on ASCII text file is incorporated herein by reference in its entirety: a computer readable form (CRF) of the Sequence Listing.

### BACKGROUND

Tissue factor (TF), also called thromboplastin, factor III, or CD142, is a transmembrane glycoprotein that functions in the coagulation pathway under normal physiological conditions. See Lwaleed et al., 2007, Biol. Res. Nurs., 9:97-107. TF is necessary for the initiation of thrombin formation from the zymogen prothrombin. Thrombin formation ultimately leads to the coagulation of blood. TF enables cells to initiate the blood coagulation cascades, and it functions as the high-affinity receptor for the coagulation factor VII (FVII), a serine protease. The resulting complex provides a catalytic event that is responsible for initiation of the coagulation protease cascades by specific limited proteolysis. Unlike the other cofactors of these protease cascades, which circulate as nonfunctional precursors, TF is a potent initiator that is fully functional when expressed on cell surfaces. In oncogenesis, TF plays a role in tumor-associated angiogenesis, progression, and metastasis. See Anand et al., 2012, Thromb. Res., 129 Suppl 1:S46-9 and Foster et al., 2006, Clin. Chim. Acta., 364:12-21. TF expression has been associated with poor clinical outcomes in solid tumors, and TF is highly expressed in cervical cancer (see Zhao et al., 2018, Exp. Ther. Med., 16:4075-81).

Cervical cancer is the fourth most common and fourth-leading cause of cancer-related death in women globally. Patients with recurrent or metastatic cervical cancer (r/mCC) have a poor prognosis, and women diagnosed with metastatic disease have a 5-year survival rate of only 17%. Doublet chemotherapy (paclitaxel-platinum or paclitaxel-topotecan) in combination with bevacizumab, if eligible, is the current first-line (1L) standard-of-care (SOC) for patients with r/mCC. See Tewari et al., 2014, N. Engl. J. Med., 370:734-43. However, the majority of patients relapse after 1L treatment and currently no SOC is established for treatment in the second-line or later (2L+) setting. Prior to the adoption of doublet chemotherapy plus bevacizumab in the 1L setting (see Miller et al., 2008, Gynecol. Oncol., 110:65-70), existing 2L+ treatment options (i.e., single-agent chemotherapy, bevacizumab) have demonstrated response rates of 4.5% to 15% and a median survival of <8 months. Data on 2L+ treatment outcomes following the current 1L SOC are limited. Single-institution studies have shown low response rates of ≤6% for 2L treatment and approximately 3% for third-line (3L) treatment. See McLachlan et al., 2017, Clin Oncol (R. Coll. Radiol.), 28:153-60. In 2018, pembrolizumab (anti-programmed death 1 antibody) received accelerated approval in the United States for the 2L+ treatment of patients with programmed death-ligand 1 (PD-L1)-positive (combined positive score ≥1%) r/mCC. The objective response rate (ORR) of pembrolizumab was 14% in this setting where 42% of patients had been previously treated with bevacizumab. See Corp. MSD. KEYTRUDA® (pembrolizumab) for injection, for intravenous use. Whitehouse Station, NJ: Merck & Co., Inc.; 06/2018. Most patients enrolled in the study had squamous histology (92%) (Id.), and therefore, little is known on the efficacy of pembrolizumab in patients with nonsquamous histology. The majority of second-line (and beyond) patients with recurrent or metastatic cervical cancer do not benefit from treatment with pembrolizumab. These data highlight the immediate need for more effective therapies that provide clinical benefits across a broader r/mCC patient population previously treated with doublet chemotherapy with or without bevacizumab and not restricted by biomarker expression.

The present invention meets this need by providing highly specific and effective anti-TF antibody-drug conjugates, in particular for the use in the treatment of cervical cancer.

All references cited herein, including patent applications, patent publications, and scientific literature, are herein incorporated by reference in their entirety, as if each individual reference were specifically and individually indicated to be incorporated by reference.

### SUMMARY

Provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject has been previously treated with bevacizumab. In some embodiments, the subject has an ECOG score of 0. In some embodiments, the subject has an ECOG score of 1. In some embodiments, the subject is less than 65 years old. In some embodiments, the subject has been previously treated with:
a) paclitaxel and cisplatin,
b) paclitaxel and carboplatin, or
c) paclitaxel and topotecan.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject has not been previously treated with bevacizumab. In some embodiments, the subject has an ECOG score of 0. In some embodiments, the subject has an ECOG score of 1. In some embodiments, the subject is less than 65 years old. In some embodiments, the subject has been previously treated with:
a) paclitaxel and cisplatin,
b) paclitaxel and carboplatin, or
c) paclitaxel and topotecan.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject has an Eastern Cooperative Oncology Group (ECOG) score of 0. In some embodiments, the subject is less than 65 years old. In some embodiments, the subject has been previously treated with:
a) paclitaxel and cisplatin,
b) paclitaxel and carboplatin, or
c) paclitaxel and topotecan.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject has an Eastern Cooperative Oncology Group (ECOG) score of 1. In some embodiments, the subject is less than 65 years old. In some embodiments, the subject has been previously treated with:
a) paclitaxel and cisplatin,
b) paclitaxel and carboplatin, or
c) paclitaxel and topotecan.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject is less than 65 years old. In some embodiments, the subject has an ECOG score of 0. In some embodiments, the subject has an ECOG score of 1. In some embodiments, the subject is less than 65 years old. In some embodiments, the subject has been previously treated with:
a) paclitaxel and cisplatin,
b) paclitaxel and carboplatin, or
c) paclitaxel and topotecan.

In some embodiments of any of the above methods or embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%. In some embodiments, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate. In some embodiments, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate. In some embodiments, the time to response is less than about 6 months after administration of the antibody-drug conjugate, optionally, wherein the time to response is less than about 4 months, about 2 months, 1.4 months, or about 1.2 months after administration of the antibody-drug conjugate.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the objective response rate is between about 13% and about 35%, optionally, wherein the objective response rate is at least about 14%, about 19%, about 21%, 23.8%, about 24%, about 25%, about 26%, about 28%, about 30%, or about 33%. In some embodiments, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate. In some embodiments, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate. In some embodiments, the time to response is less than about 6 months after administration of the antibody-drug conjugate, optionally, wherein the time to response is less than about 4 months, about 2 months, 1.4 months, or about 1.2 months after administration of the antibody-drug conjugate.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%. In some embodiments, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate. In some embodiments, the time to response is less than about 6 months after administration of the antibody-drug conjugate, optionally, wherein the time to response is less than about 4 months, about 2 months, 1.4 months, or about 1.2 months after administration of the antibody-drug conjugate.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%. In some embodiments, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate. In some embodiments, the time to response is less than about 6 months after administration of the antibody-drug conjugate, optionally, wherein the time to response is less than about 4 months, about 2 months, 1.4 months, or about 1.2 months after administration of the antibody-drug conjugate.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate. In some embodiments, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate. In some embodiments, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%. In some embodiments, the time to response is less than about 6 months after administration of the antibody-drug conjugate, optionally, wherein the time to response is less than about 4 months, about 2 months, 1.4 months, or about 1.2 months after administration of the antibody-drug conjugate.

Also provided herein is a method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the time to response is less than about 6 months after administration of the antibody-drug conjugate, optionally, wherein the time to response is less than about 4 months, about 2 months, 1.4 months, or about 1.2 months after administration of the antibody-drug conjugate. In some embodiments, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate. In some embodiments, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate. In some embodiments, the objective response rate is at least about between about 13% and about 35%, optionally, wherein the objective response rate is at least about 14%, about 19%, about 21%, 23.8%, about 24%, about 25%, about 26%, about 28%, about 30%, or about 33%.

In some embodiments of any of the above methods or embodiments, the subject has been previously treated with bevacizumab. In some embodiments, the subject has not been previously treated with bevacizumab. In some embodiments, the subject has experienced disease progression during or after treatment with:
a) paclitaxel and cisplatin,
b) paclitaxel and carboplatin,
c) paclitaxel and topotecan,
d) bevacizumab, paclitaxel, and cisplatin,
e) bevacizumab, paclitaxel, and carboplatin, or
f) bevacizumab, paclitaxel, and topotecan. In some embodiments, the subject is less than 65 years old. In some embodiments, the subject has an ECOG score of 0. In some embodiments, the subject has an ECOG score of 1. In some embodiments, the cervical cancer is an adenocarcinoma. In some embodiments, the cervical cancer is an adenosquamous carcinoma. In some embodiments, the cervical cancer is a squamous cell carcinoma. In some embodiments, the cervical cancer is a non-squamous cell carcinoma. In some embodiments, the cervical cancer is recurrent or metastatic cervical cancer. In some embodiments, the subject has been previously treated with one or more therapeutic agents and did not respond to the treatment, wherein the one or more therapeutic agents is not the antibody-drug conjugate. In some embodiments, the subject has been previously treated with one or more therapeutic agents and relapsed after the treatment, wherein the one or more therapeutic agents is not the antibody-drug conjugate. In some embodiments, the subject has been previously treated with one or more therapeutic agents and has experienced disease progression during treatment, wherein the one or more therapeutic agents is not the antibody-drug conjugate. In some embodiments, the one or more therapeutic agents is a platinum-based therapeutic agent. In some embodiments, the one or more therapeutic agents is selected from the group consisting of: paclitaxel, cisplatin, carboplatin, topotecan, gemcitabine, fluorouracil, ixabepilone, imatinib mesylate, docetaxel, gefitinib, nab-paclitaxel, pemetrexed, vinorelbine, doxil, cetuximab, pembrolizumab, nivolumab and bevacizumab. In some embodiments, the subject is not a candidate for curative therapy. In some embodiments, the curative therapy comprises radiotherapy and/or exenterative surgery. In some embodiments, the subject has received prior radiation to the pelvis. In some embodiments, the subject has not received prior radiation to the pelvis. In some embodiments, the subject has received one prior line of systemic therapy for relapsed, recurrent or metastatic cancer. In some embodiments, the subject has received two prior lines of systemic therapy for relapsed, recurrent or metastatic cancer. In some embodiments, the subject did not respond to treatment with the prior systemic therapy. In some embodiments, the subject relapsed after treatment with the prior systemic therapy. In some embodiments, the cervical cancer is an advanced stage cervical cancer, such as a stage 3 or stage 4 cervical cancer, such as metastatic cervical cancer. In some embodiments, the cervical cancer is recurrent cervical cancer. In some embodiments, the route of administration for the antibody-drug conjugate is intravenous. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF. In some embodiments, the subject has a TF histology score (H-score) of at least 1. In some embodiments, the subject has one or more adverse events and is further administered an additional therapeutic agent to eliminate or reduce the severity of the one or more adverse events. In some embodiments, the subject is at risk of developing one or more adverse events and is further administered an additional therapeutic agent to prevent or reduce the severity of the one or more adverse events. In some embodiments, the subject has one or more adverse events and the dose of the antibody drug conjugate is reduced following the one or more adverse events. In some embodiments, the dose is reduced from 2.0 mg/kg to 1.3 mg/kg. In some embodiments, the dose is reduced from 1.3 mg/kg to 0.9 mg/kg. In some embodiments, the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration. In some embodiments, the one or more adverse events is a grade 3 or greater adverse event. In some embodiments, the one or more adverse events is a serious adverse event. In some embodiments, the one or more adverse events is conjunctivitis and/or keratitis and the additional agent is a preservative-free lubricating eye drop, an ocular vasoconstrictor and/or a steroid eye drop. In some embodiments, the antibody-drug conjugate is administered as a monotherapy. In some embodiments, the subject is a human. In some embodiments, the antibody-drug conjugate is in a pharmaceutical composition comprising the antibody-drug conjugate and a pharmaceutical acceptable carrier.

Also provided herein is an antibody-drug conjugate that binds to TF for use in any of the methods or embodiments provided herein.

Also provided herein is the use of an antibody-drug conjugate that binds to TF for the manufacture of a medicament for use in any of the methods or embodiments provided herein.

### BRIEF DESCRIPTION OF THE DRAWINGS

FIG. 1 is a diagram showing the mechanism of action (MOA) of the antibody-drug conjugate tisotumab vedotin.

FIG. 2 is a diagram showing the Phase II study design for treatment with tisotumab vedotin in patients with previously treated, recurrent or metastatic cancer who have received at least one prior line of systemic therapy. ^{a} indicates tisotumab vedotin 2.0 mg/kg infusion on day 1 of each cycle until disease progression. Each treatment cycle was 3 weeks (Q3W). ^{b} indicates CT or MRI scan every 6 weeks (±7 days) for the first 30 weeks of treatment and every 12 weeks (±7 days) thereafter regardless of treatment delays.

FIG. 3 is a patient flowchart and disposition of the Phase II study.

FIG. 4 is a diagram showing target lesion response in each subject. Bars indicates the best percent change in lesion size relative to baseline for each subject.

FIG. 5 is a graph showing the percent of subjects remaining in response over time.

FIG. 6 is a graph showing the percent of subjects exhibiting progression free survival over time.

FIG. 7 is a graph showing the percent of subjects surviving over time.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “tissue factor”, “TF”, “CD142”, “tissue factor antigen”, “TF antigen” and “CD142 antigen” are used interchangeably herein, and, unless specified otherwise, include any variants, isoforms and species homologs of human tissue factor which are naturally expressed by cells or are expressed on cells transfected with the tissue factor gene. Tissue factor may be the sequence Genbank accession NP_001984.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to TF is substantially free of antibodies that bind specifically to antigens other than TF). An isolated antibody that binds specifically to TF can, however, have cross-reactivity to other antigens, such as TF molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-TF antibody includes a conjugate of an anti-TF antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-TF antibody is an antibody that binds to the antigen TF. In another example, an anti-VEGF antibody is an antibody that binds to the antigen VEGF.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “k_{d}” (sec⁻¹), as used herein, refers to the dissociation rate constant of a particular antibody-antigen interaction. Said value is also referred to as the k_{off} value.

The term “kₐ” (M⁻¹×sec⁻¹), as used herein, refers to the association rate constant of a particular antibody-antigen interaction.

The term “K_{A}” (M⁻¹), as used herein, refers to the association equilibrium constant of a particular antibody-antigen interaction and is obtained by dividing the kₐ by the k_{d}.

The term “ADC” refers to an antibody-drug conjugate, which in the context of the present invention refers to an anti-TF antibody, which is coupled to another moiety (e.g., MMAE or MMAF) as described in the present application.

The abbreviations “vc” and “val-cit” refer to the dipeptide valine-citrulline.

The abbreviation “PAB” refers to the self-immolative spacer:

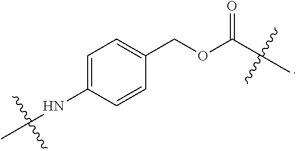 [SMILES: *Nc1ccc(COC(*)=O)cc1]

The abbreviation “MC” refers to the stretcher maleimidocaproyl:

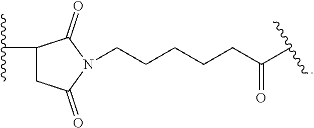 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a cervical cancer refers to a tumor that is the result of a metastasized cervical cancer.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” refers to an amount effective, at dosages and for periods of time necessary, to achieve a desired therapeutic result. Such desired therapeutic results include protecting a subject against the onset of a disease or promoting disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. A therapeutically effective amount of an anti-TF antibody-drug conjugate may vary according to factors such as the disease state, age, sex, and weight of the individual, and the ability of the anti-TF antibody-drug conjugate to elicit a desired response in the individual. A therapeutically effective amount is also one in which any toxic or detrimental effects of the anti-TF antibody-drug conjugate are outweighed by the therapeutically beneficial effects.

A therapeutically effective amount of a drug (e.g., anti-TF antibody-drug conjugate) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an antibody-drug conjugate) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for “progressive disease” or “PD”, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “objective response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a patient is calculated based on the weight of the patient. For example, when a patient with 60 kg body weight requires 2 mg/kg of an anti-TF antibody-drug conjugate, one can calculate and use the appropriate amount of the anti-TF antibody-drug conjugate (i.e., 120 mg) for administration.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-TF antibody-drug conjugate). For example, a 60 kg person and a 100 kg person would receive the same dose of an antibody-drug conjugate (e.g., 240 mg of an anti-TF antibody-drug conjugate).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” refers to the physical introduction of a therapeutic agent to a subject, using any of the various methods and delivery systems known to those skilled in the art. Exemplary routes of administration for the anti-TF antibody-drug conjugate include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “baseline” or “baseline value” used interchangeably herein can refer to a measurement or characterization of a symptom before the administration of the therapy (e.g., an antibody-drug conjugate as described herein) or at the beginning of administration of the therapy. The baseline value can be compared to a reference value in order to determine the reduction or improvement of a symptom of a TF-associated disease contemplated herein (e.g., cervical cancer). The terms “reference” or “reference value” used interchangeably herein can refer to a measurement or characterization of a symptom after administration of the therapy (e.g., an antibody-drug conjugate as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the antibody drug conjugate is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically important”
Requires inpatient hospitalization or prolongation of existing hospitalization, excluding the following: 1) routine treatment or monitoring of the underlying disease, not associated with any deterioration in condition, 2) elective or pre-planned treatment for a pre-existing condition that is unrelated to the indication under study and has not worsened since signing the informed consent, and social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” “once about every three weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Antibody-Drug Conjugates

The present invention provides for anti-TF antibody-drug conjugates that are useful for the treatment of cancer in a subject. In some embodiments, the cancer is cervical cancer. In some embodiments, the cervical cancer is an advanced stage cervical cancer (e.g., stage 3 cervical cancer or stage 4 cervical cancer or metastatic cervical cancer). In some embodiments, the advanced cervical cancer is a metastatic cancer. In some embodiments, the subject has relapsed, recurrent and/or metastatic cervical cancer. In some embodiments the anti-TF antibody-drug conjugate is tisotumab vedotin. As described in the Phase II clinical trial in the Example herein, tisotumab vedotin is effective for the treatment of relapsed, recurrent and/or metastatic cervical cancer. Responses to treatment were observed in various subgroups of interest, including cancer histology, line of therapy, previous treatment with cisplatin and radiation, previous treatment with bevacizumab in combination with chemotherapy doublet, and ECOG performance status. Surprisingly, tisotumab vedotin was able to effectively treat subjects that had previously been treated with bevacizumab, which has not been demonstrated for other treatments, such as pembrolizumab. Tisotumab vedotin was also effective in treating both squamous and non-squamous cervical cancers, whereas pembrolizumab has not been demonstrated to be effective for the treatment of non-squamous cervical cancers. Furthermore, treatment with tisotumab vedotin resulted in a clinically meaningful confirmed ORR per IRC of 24% (CI: 15.9%-33.3%) in subjects with previously treated recurrent or metastatic cervical cancer, with 7 subjects achieving a CR (6.9%). Responses were durable, with a median DOR of 8.3 months [95% CI 4.3, NR]. Overall survival was 12.1 months. Additionally, an estimated 67% of responses to tisotumab vedotin remain ongoing at 6 months. Results for other monotherapy treatments for 2L+ cervical cancers are summarized in the following table:

| | | | | | Median | Median | Median |
|---|---|---|---|---|---|---|---|
| Study | | Number | ORR %* | | DOR | PFS | Os |
| reference | Intervention | of patients | (95% CI) | CR | (months) | (months) | (months) |
| Alberts 2012 | Nab-Paclitaxel | 35 | 28.6 (14.6- | 0 | 6 | 5 | 9.4 |
| | | | 46.3) | | | | |
| Garcia 2007 | Docetaxel | 23 | 8.7 (1.1- | 0 | 4.7 | 3.8 | 7 |
| | | | 28.0) | | | | |
| Angioli 2007 | Liposomal | 10 | 10 (0.3- | 0 | NR | NR | NR |
| | doxorubicin | | 44.5) | | | | |
| Rose 2006 | Liposomal | 27 | 11.1 (2.4- | 0 | 2.9 | 3.2 | 8.9 |
| | doxorubicin | | 29.2) | | | | |
| Coronel 2009 | Topotecan | 18 | 0 | 0 | NA | 3.5 | 7 |
| Fiorica 2009 | Topotecan | 25 | 0 | 0 | NA | 2.4 (BOR | NR |
| | | | | | | PD) | |
| | | | | | | 6.2 (BOR | |
| | | | | | | SD) | |
| Miller 2008 | Pemetrexed | 27 | 14.8 (4.2- | 0 | 4.4 | 3.1 | 7.4 |
| | | | 33.7) | | | | |
| Schilder 2005 | Gemcitabine | 22 | 4.5 (0.1- | 0 | 4.7 | 2.1 | 6.5 |
| | | | 22.8) | | | | |
| Schilder 2009 | Erlotinib | 25 | 0 | 0 | NA | 1.9 | 5.0 |
| Monk 2009 | Bevacizumab | 46 | 10.9 (3.6- | 0 | 6.2 | 3.4 | 7.3 |
| | | | 23.6) | | | | |
| Chung 2019 | Pembrolizumab | 98 | 12.2 (All) | 3 | Not | 2.1 (All) | 9.4 (All) |
| | | | (6.5-20.4) | (3.1%) | reached | | |
| | | | 14.3 | | | | |
| | | | (n = 77) | | | | |
| | | | (7.4-24.1) | | | | |
| | | | N = 77: PD- | | | | |
| | | | L1+, prev. | | | | |
| | | | treated | | | | |
| Verschraegen | Irinotecan | 42 | 21 (NR) | NR | 3 | NR | 6.4 |
| 1997 | | | | | | | |
| Bookman | Topotecan | 45 | 12.5 (NR) | NR | 4.0 | 2.1 | 6.6 |
| 2000 | | | | | | | |
| Muggia 2004 | Vinorelbine | 44 | 13.7 (5.2- | NR | NR | NR | NR |
| | | | 27.4) | | | | |

NR = not reported; NA = not applicable
Citations: Alberts, et al., (2012) Gynecol Oncol. 127(3): 451-5; Garcia, et al., (2007) Am J Clin Oncol. 30: 428-31; Angioli et al., (2007) Int J Gynecol Cancer 17(1): 88-93; Rose, et al. (2006) Gynecol. Oncol. 102(2): 210-3; Coronel, et al. (2009) Med Oncol. 26(2): 210-4; Fiorica, et al. (2009) Gynecol Oncol. 115(2): 285-9; Schilder, et al., (2005) Gynecol Oncol 96: 103-7; Schilder, et al. (2009) Int J Gynecol Cancer 19(5): 929-33; Monk, et al., (2009) J Clin Oncol. 27: 1069-1074; Chung, et al. (2019) J. Clin. Oncol. 37(17): 1470-78; Verschraegen, et al., (1997) (2): 625-31; Bookman, et al., (2000) Gynecol Oncol. 77: 446-9; Muggia et al., (2004) Gynecol Oncol. 92: 639-43.

A. Anti-TF Antibody

Generally, anti-TF antibodies of the disclosure bind TF, e.g., human TF, and exert cytostatic and cytotoxic effects on malignant cells, such as cervical cancer cells. Anti-TF antibodies of the disclosure are preferably monoclonal, and may be multispecific, human, humanized or chimeric antibodies, single chain antibodies, Fab fragments, F(ab′) fragments, fragments produced by a Fab expression library, and TF binding fragments of any of the above. In some embodiments, the anti-TF antibodies of the disclosure specifically bind TF. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-TF antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-TF antibodies or antigen-binding fragments thereof are human, murine (e.g., mouse and rat), donkey, sheep, rabbit, goat, guinea pig, camelid, horse, or chicken.

Anti-TF antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 June 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

CDR sequences of the anti-TF antibodies of the anti-TF antibody-drug conjugates provided herein are according to the IMGT numbering scheme as described in Lefranc, M. P. et al., Dev. Comp. Immunol., 2003, 27, 55-77.

In certain embodiments antibodies of the disclosure comprise one or more CDRs of the antibody 011. See WO 2011/157741 and WO 2010/066803. The disclosure encompasses an antibody or derivative thereof comprising a heavy or light chain variable domain, said variable domain comprising (a) a set of three CDRs, in which said set of CDRs are from monoclonal antibody 011, and (b) a set of four framework regions, in which said set of framework regions differs from the set of framework regions in monoclonal antibody 011, and in which said antibody or derivative thereof binds to TF. In some embodiments, said antibody or derivative thereof specifically binds to TF. In certain embodiments, the anti-TF antibody is 011. The antibody 011 is also known as tisotumab.

In one aspect, anti-TF antibodies that compete with tisotumab binding to TF are also provided herein. Anti-TF antibodies that bind to the same epitope as tisotumab are also provided herein.

In one aspect, provided herein is an anti-TF antibody comprising 1, 2, 3, 4, 5, or 6 of the CDR sequences of tisotumab.

In one aspect, provided herein is an anti-TF antibody comprising a heavy chain variable region and a light chain variable region, wherein the heavy chain variable region comprises (i) CDR-H1 comprising the amino acid sequence of SEQ ID NO: 1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6, wherein the CDRs of the anti-TF antibody are defined by the IMGT numbering scheme.

An anti-TF antibody described herein may comprise any suitable framework variable domain sequence, provided that the antibody retains the ability to bind TF (e.g., human TF). As used herein, heavy chain framework regions are designated “HC-FR1-FR4,” and light chain framework regions are designated “LC-FR1-FR4.” In some embodiments, the anti-TF antibody comprises a heavy chain variable domain framework sequence of SEQ ID NO:9, 10, 11, and 12 (HC-FR1, HC-FR2, HC-FR3, and HC-FR4, respectively). In some embodiments, the anti-TF antibody comprises a light chain variable domain framework sequence of SEQ ID NO: 13, 14, 15, and 16 (LC-FR1, LC-FR2, LC-FR3, and LC-FR4, respectively).

In some embodiments of the anti-TF antibodies described herein, the heavy chain variable domain comprises the amino acid sequence of

EVQLLESGGGLVQPGGSLRLSCAASGFTFSNYAMSWVRQAPGKGLEWVSSISGSGDYT YYTDSVKGRFTISRDNSKNTLYLQMNSLRAEDTAVYYCARSPWGYYLDSWGQGTLVT VSS (SEQ ID NO:7) and the light chain variable domain comprises the amino acid sequence of DIQMTQSPPSLSASAGDRVTITCRASQGISSRLAWYQQKPEKAPKSLIYAASSLQSGVPSR FSGSGSGTDFTLTISSLQPEDFATYYCQQYNSYPYTFGQGTKLEIK (SEQ ID NO:8).

In some embodiments of the anti-TF antibodies described herein, the heavy chain CDR sequences comprise the following:

a) CDR-H1
(SEQ ID NO: 1)
GFTFSNYA;
b) CDR-H2
(SEQ ID NO: 2)
ISGSGDYT;
and
c) CDR-H3
(SEQ ID NO: 3)
ARSPWGYYLDS.

In some embodiments of the anti-TF antibodies described herein, the heavy chain FR sequences comprise the following:

a) HC-FR1
(SEQ ID NO: 9)
EVQLLESGGGLVQPGGSLRLSCAAS;
b) HC-FR2
(SEQ ID NO: 10)
MSWVRQAPGKGLEWVSS;
c) HC-FR3
(SEQ ID NO: 11)
YYTDSVKGRFTISRDNSKNTLYLQMNSLRAEDTAVYYC;
and
d) HC-FR4
(SEQ ID NO: 12)
WGQGTLVTVSS.

In some embodiments of the anti-TF antibodies described herein, the light chain CDR sequences comprise the following:

a) CDR-L1
(SEQ ID NO: 4)
QGISSR;
b) CDR-L2
(SEQ ID NO: 5)
AAS;
and
c) CDR-L3
(SEQ ID NO: 6)
QQYNSYPYT.

In some embodiments of the anti-TF antibodies described herein, the light chain FR sequences comprise the following:

a) LC-FR1
(SEQ ID NO: 13)
DIQMTQSPPSLSASAGDRVTITCRAS;
b) LC-FR2
(SEQ ID NO: 14)
LAWYQQKPEKAPKSLIY;
c) LC-FR3
(SEQ ID NO: 15)
SLQSGVPSRFSGSGSGTDFTLTISSLQPEDFATYYC;
and
d) LC-FR4
(SEQ ID NO: 16)
FGQGTKLEIK.

In some embodiments, provided herein is an anti-TF antibody that binds to TF (e.g., human TF), wherein the antibody comprises a heavy chain variable region and a light chain variable region, wherein the antibody comprises:
(a) heavy chain variable domain comprising:
   (1) an HC-FR1 comprising the amino acid sequence of SEQ ID NO: 9;
   (2) an CDR-H1 comprising the amino acid sequence of SEQ ID NO: 1;
   (3) an HC-FR2 comprising the amino acid sequence of SEQ ID NO: 10;
   (4) an CDR-H2 comprising the amino acid sequence of SEQ ID NO: 2;
   (5) an HC-FR3 comprising the amino acid sequence of SEQ ID NO: 11;
   (6) an CDR-H3 comprising the amino acid sequence of SEQ ID NO: 3; and
   (7) an HC-FR4 comprising the amino acid sequence of SEQ ID NO: 12, and/or
(b) a light chain variable domain comprising:
   (1) an LC-FR1 comprising the amino acid sequence of SEQ ID NO: 13;
   (2) an CDR-L1 comprising the amino acid sequence of SEQ ID NO:4;
   (3) an LC-FR2 comprising the amino acid sequence of SEQ ID NO: 14;
   (4) an CDR-L2 comprising the amino acid sequence of SEQ ID NO: 5;
   (5) an LC-FR3 comprising the amino acid sequence of SEQ ID NO: 15;
   (6) an CDR-L3 comprising the amino acid sequence of SEQ ID NO: 6; and
   (7) an LC-FR4 comprising the amino acid sequence of SEQ ID NO: 16.

In one aspect, provided herein is an anti-TF antibody comprising a heavy chain variable domain comprising the amino acid sequence of SEQ ID NO:7 or comprising a light chain variable domain comprising the amino acid sequence of SEQ ID NO:8. In one aspect, provided herein is an anti-TF antibody comprising a heavy chain variable domain comprising the amino acid sequence of SEQ ID NO:7 and comprising a light chain variable domain comprising the amino acid sequence of SEQ ID NO: 8.

In some embodiments, provided herein is an anti-TF antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO: 1, (b) CDR-H2 comprising the amino acid sequence of SEQ ID NO:2, and (c) CDR-H3 comprising the amino acid sequence of SEQ ID NO:3.

In some embodiments, provided herein is an anti-TF antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a light chain variable domain sequence of SEQ ID NO:8 including post-translational modifications of that sequence. In a particular embodiment, the light chain variable domain comprises one, two or three CDRs selected from: (a) CDR-L1 comprising the amino acid sequence of SEQ ID NO:4, (b) CDR-L2 comprising the amino acid sequence of SEQ ID NO:5, and (c) CDR-L3 comprising the amino acid sequence of SEQ ID NO:6.

In some embodiments, the anti-TF antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-TF antibody of the anti-TF antibody-drug conjugate comprises: i) a heavy chain CDR1 comprising the amino acid sequence of SEQ ID NO: 1, a heavy chain CDR2 comprising the amino acid sequence of SEQ ID NO: 2, a heavy chain CDR3 comprising the amino acid sequence of SEQ ID NO: 3; and ii) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 6, wherein the CDRs of the anti-TF antibody are defined by the IMGT numbering scheme.

In some embodiments, the anti-TF antibody of the anti-TF antibody-drug conjugate comprises: i) an amino acid sequence having at least 85% sequence identity to a heavy chain variable region comprising the amino acid sequence of SEQ ID NO: 7, and ii) an amino acid sequence having at least 85% sequence identity to a light chain variable region comprising the amino acid sequence of SEQ ID NO: 8.

In some embodiments, the anti-TF antibody of the anti-TF antibody-drug conjugate comprises: i) an amino acid sequence having at least 85% sequence identity to a heavy chain comprising the amino acid sequence of SEQ ID NO: 17, and ii) an amino acid sequence having at least 85% sequence identity to a light chain comprising the amino acid sequence of SEQ ID NO: 18.

In one embodiment, the antibody comprises the heavy chain sequence of SEQ ID NO: 17 and the light chain sequence of SEQ ID NO:18, including post-translational modifications of those sequences. In one embodiment, the antibody comprises the heavy chain sequence of SEQ ID NO:17 and the light chain sequence of SEQ ID NO:18.

In some embodiments, the anti-TF antibody of the anti-TF antibody-drug conjugate is a monoclonal antibody.

In some embodiments, the anti-TF antibody of the anti-TF antibody-drug conjugate is tisotumab, which is also known as antibody 011 as described in WO 2011/157741 and WO 2010/066803.

Anti-TF antibodies of the present invention may also be described or specified in terms of their binding affinity to TF (e.g., human TF). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to TF or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

| Antibody Region | Amino Acid Sequence |
|---|---|
| CDR-H1 | GFTFSNYA (SEQ ID NO: 1) |
| CDR-H2 | ISGSGDYT (SEQ ID NO: 2) |
| CDR-H3 | ARSPWGYYLDS (SEQ ID NO: 3) |
| CDR-L1 | QGISSR (SEQ ID NO: 4) |
| CDR-L2 | AAS (SEQ ID NO: 5) |
| CDR-L3 | QQYNSYPYT (SEQ ID NO: 6) |
| Heavy chain | EVQLLESGGGLVQPGGSLRLSCAASGFTFSNYAMSWVRQA |
| variable domain | PGKGLEWVSSISGSGDYTYYTDSVKGRFTISRDNSKNTLYL |
| (VH) | QMNSLRAEDTAVYYCARSPWGYYLDSWGQGTLVTVSS (SEQ ID NO: 7) |
| Light chain | DIQMTQSPPSLSASAGDRVTITCRASQGISSRLAWYQQKPE |
| variable domain | KAPKSLIYAASSLQSGVPSRFSGSGSGTDFTLTISSLQPEDF |
| (VL) | ATYYCQQYNSYPYTFGQGTKLEIK (SEQ ID NO: 8) |
| HC-FR1 | EVQLLESGGGLVQPGGSLRLSCAAS (SEQ ID NO: 9) |
| HC-FR2 | MSWVRQAPGKGLEWVSS (SEQ ID NO: 10) |
| HC-FR3 | YYTDSVKGRFTISRDNSKNTLYLQMNSLRAEDTAVYYC (SEQ ID NO: 11) |
| HC-FR4 | WGQGTLVTVSS (SEQ ID NO: 12) |
| LC-FR1 | DIQMTQSPPSLSASAGDRVTITCRAS (SEQ ID NO: 13) |
| LC-FR2 | LAWYQQKPEKAPKSLIY (SEQ ID NO: 14) |
| LC-FR3 | SLQSGVPSRFSGSGSGTDFTLTISSLQPEDFATYYC (SEQ ID NO: 15) |
| LC-FR4 | FGQGTKLEIK (SEQ ID NO: 16) |
| Heavy chain | EVQLLESGGG LVQPGGSLRL SCAASGFTFS NYAMSWVRQA |
| | PGKGLEWVSS ISGSGDYTYY TDSVKGRFTI SRDNSKNTLY |
| | LQMNSLRAED TAVYYCARSP WGYYLDSWGQ GTLVTVSSAS |
| | TKGPSVFPLA PSSKSTSGGT AALGCLVKDY FPEPVTVSWN |
| | SGALTSGVHT FPAVLQSSGL YSLSSVVTVP SSSLGTQTYI CNVNHKPSNT |
| | KVDKRVEPKS CDKTHTCPPC PAPELLGGPS VFLFPPKPKD TLMISRTPEV |
| | TCVVVDVSHE DPEVKFNWYV DGVEVHNAKT KPREEQYNST |
| | YRVVSVLTVL HQDWLNGKEY KCKVSNKALP APIEKTISKA |
| | KGQPREPQVY TLPPSREEMT KNQVSLTCLV KGFYPSDIAV |
| | EWESNGQPEN NYKTTPPVLD SDGSFFLYSK LTVDKSRWQQ |
| | GNVFSCSVMH EALHNHYTQK SLSLSPGK (SEQ ID NO: 17) |
| Light chain | DIQMTQSPPS LSASAGDRVT ITCRASQGIS SRLAWYQQKP EKAPKSLIYA |
| | ASSLQSGVPS RFSGSGSGTD FTLTISSLQP EDFATYYCQQ YNSYPYTFGQ |
| | GTKLEIKRTV AAPSVFIFPP SDEQLKSGTA SVVCLLNNFY |
| | PREAKVQWKV DNALQSGNSQ ESVTEQDSKD STYSLSSTLT |
| | LSKADYEKHK VYACEVTHQG LSSPVTKSFN RGEC (SEQ ID NO: 18) |

B. Antibody-Drug Conjugate Structure

In some aspects, the anti-TF antibody-drug conjugates described herein comprise a linker between an anti-TF antibody or antigen-binding fragment thereof as described herein and a cytostatic or cytotoxic drug. In some embodiments the linker is a non-cleavable linker. In some embodiments the linker is a cleavable linker.

In some embodiments, the linker is a cleavable peptide linker comprising maleimido caproyl (MC), the dipeptide valine-citrulline (vc) and p-aminobenzylcarbamate (PAB). In some embodiments, the cleavable peptide linker has the formula: MC-vc-PAB-, wherein:
a) MC is:

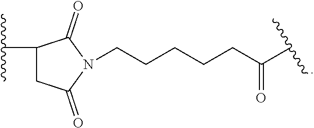 [SMILES: *C(=O)CCCCCN1C(=O)CC(*)C1=O]

b) vc is the dipeptide valine-citrulline, and
c) PAB is:

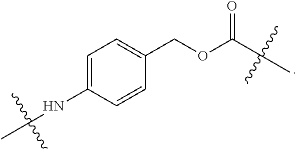 [SMILES: *Nc1ccc(COC(*)=O)cc1]

In some embodiments, the linker is a cleavable peptide linker comprising maleimido caproyl (MC). In some embodiments, the cleavable peptide linker has the formula: MC-, wherein:
a) MC is:

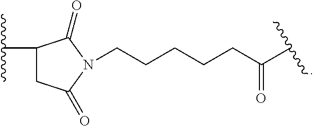 [SMILES: *C(=O)CCCCCN1C(=O)CC(*)C1=O]

In some embodiments, the linker is attached to sulphydryl residues of the anti-TF antibody or antigen-binding fragment thereof obtained by partial or full reduction of the anti-TF antibody or antigen-binding fragment thereof. In some embodiments, the linker is attached to sulphydryl residues of the anti-TF antibody or antigen-binding fragment thereof obtained by partial reduction of the anti-TF antibody or antigen-binding fragment thereof. In some embodiments, the linker is attached to sulphydryl residues of the anti-TF antibody or antigen-binding fragment thereof obtained by full reduction of the anti-TF antibody or antigen-binding fragment thereof.

In some aspects, the anti-TF antibody-drug conjugates described herein comprise a linker as described herein between an anti-TF antibody or antigen-binding fragment thereof as described herein and a cytostatic or cytotoxic drug. Auristatins have been shown to interfere with microtubule dynamics, GTP hydrolysis and nuclear and cellular division (See Woyke et al (2001) Antimicrob. Agents and Chemother. 45(12): 3580-3584) and have anti-cancer (See U.S. Pat. Nos. 5663149) and antifungal activity (See Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023. In some embodiments of the anti-TF antibody-drug conjugates described herein, the cytostatic or cytotoxic drug is an auristatin or a functional analog thereof (e.g., functional peptide thereof) or a functional derivative thereof. In some embodiments, the auristatin is a monomethyl auristatin or a functional analog thereof (e.g., functional peptide thereof) or a functional derivative thereof.

In one embodiment, the auristatin is monomethyl auristatin E (MMAE):

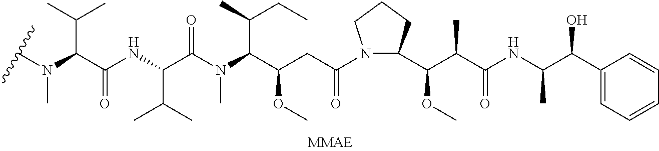 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

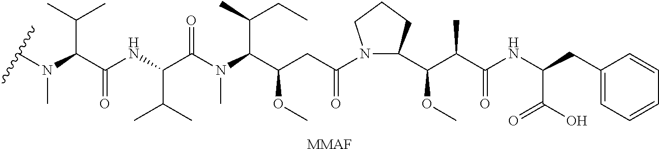

wherein the wavy line indicates the attachment site for the linker.

In one embodiment, the cleavable peptide linker has the formula: MC-vc-PAB-, and is attached to MMAE. The resulting linker-auristatin, MC-vc-PAB-MMAE is also designated vcMMAE. The vcMMAE drug linker moiety and conjugation methods are disclosed in WO2004010957, U.S. Pat. Nos. 7,659,241, 7,829,531 and 7,851,437. When vcMMAE is attached to an anti-TF antibody or antigen-binding fragment thereof as described herein, the resulting structure is:

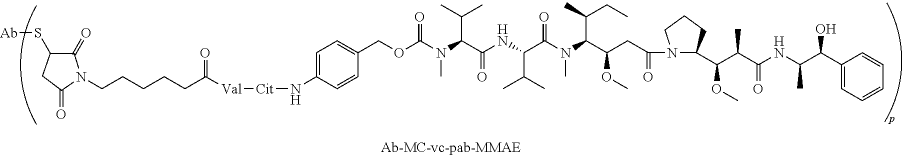

wherein p denotes a number from 1 to 8, e.g., 1, 2, 3, 4, 5, 6, 7 or 8, e.g., p may be from 3-5, S represents a sulphydryl residue of the anti-TF antibody and Ab designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the cleavable peptide linker has the formula: MC-vc-PAB-, and is attached to MMAF. The resulting linker-auristatin, MC-vc-PAB-MMAF is also designated vcMMAF. In another embodiment, a non-cleavable linker MC is attached to MMAF. The resulting linker-auristatin MC-MMAF is also designated mcMMAF. Both the vcMMAF and mcMMAF drug linker moieties and conjugation methods are disclosed in WO2005081711 and U.S. Pat. No. 7,498,298. When vcMMAF or mcMMAF is attached to an anti-TF antibody or antigen-binding fragment thereof as described herein, the resulting structure is:

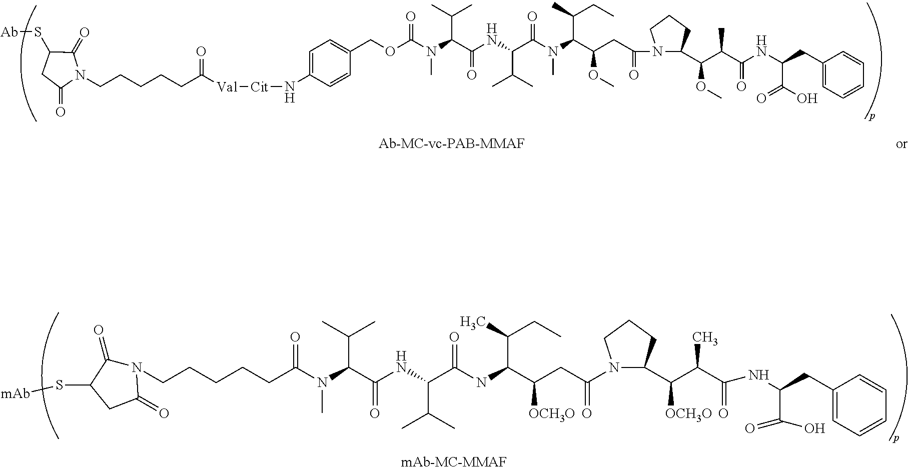

wherein p denotes a number from 1 to 8, e.g., 1, 2, 3, 4, 5, 6, 7 or 8, e.g., p may be from 3-5, S represents a sulphydryl residue of the anti-TF antibody and Ab or mAb designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the antibody-drug conjugate is tisotumab vedotin or a biosimilar thereof.

In one embodiment, the antibody-drug conjugate is tisotumab vedotin.

C. Nucleic Acids, Host Cells and Methods of Production

In some aspects, also provided herein are nucleic acids encoding an anti-TF antibody or antigen-binding fragment thereof as described herein. Further provided herein are vectors comprising the nucleic acids encoding an anti-TF antibody or antigen-binding fragment thereof as described herein. Further provided herein are host cells expressing the nucleic acids encoding an anti-TF antibody or antigen-binding fragment thereof as described herein. Further provided herein are host cells comprising the vectors comprising the nucleic acids encoding an anti-TF antibody or antigen-binding fragment thereof as described herein. Methods of producing the anti-TF antibody, linker and antibody-drug conjugate are described in U.S. Pat. No. 9,168,314.

The anti-TF antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, 5,891,693.

After isolating and purifying the antibodies from the cell media using well known techniques in the art, they are conjugated with an auristatin via a linker as described in U.S. Pat. No. 9,168,314.

Monoclonal anti-TF antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., JMol, Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody of the invention is a human antibody. Human monoclonal antibodies directed against tissue factor may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and κ light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG, κ monoclonal antibodies (Lonberg, N. et al. (1994), supra; reviewed in Lonberg, N. Handbook of Experimental Pharmacology 113, 49-101 (1994), Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a −460 kb yeast artificial chromosome fragment of the yIgH24 chromosome. This line was designated (HCo17) 25950. The (HCo17) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain trans genes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pV×6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KCO5) 9272 trans gene (Fishwild eta). (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KCO05 [MIK] (Balb) mice which were generated by backcrossing the KC05 strain (as described in Fishwild et (1996) Nature Biotechnology, 14:845-851) to wild-type Balb/c mice to generate mice as described in WO09097006. Using this crossing Balb/c hybrids were created for HCo12, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques, Human monoclonal or polyclonal antibodies of the present invention, or antibodies of the present invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the present invention or antibodies of the present invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### III. Method of Treatment

A. Cervical Cancer

Cervical cancer remains to be one of the leading causes of cancer-related death in women despite advances in screening, diagnosis, prevention, and treatment. It accounts for −4% of the total newly diagnosed cancer cases and 4% of the total cancer deaths. See Zhu et al., 2016, Drug Des. Devel. Ther. 10:1885-1895. Cervical cancer is the 7^{th} most common female cancer worldwide and the 16^{th} most common cancer in the European Union. Depending on the stage at initial presentation, cervical cancer will recur in 25-61% of women. See Tempfer et al., 2016, Oncol. Res. Treat. 39:525-533. In most cases, recurrent disease is diagnosed within 2 years of the initial treatment and may be observed in various sites. Chemotherapy is the standard treatment for these patients. See Zhu et al., 2016, Drug Des. Devel. Ther. 10:1885-1895. The median overall survival exceeds one year now, however, the five year relative survival for stage IV cervical cancer is only 15%, demonstrating the high need for improved methods of treating cervical cancer.

The invention provides methods for treating cervical cancer with an antibody-drug conjugate described herein. In a preferred aspect, the antibody-drug conjugate is tisotumab vedotin. In one aspect, the antibody-drug conjugates described herein are for use in a method of treating cervical cancer in a subject. In some embodiments, the subject has not previously received treatment for the cervical cancer. In some embodiments, the subject has received at least one previous treatment for the cervical cancer. In some embodiments, the subject was previously treated with bevacizumab. In some embodiments, the subject has been previously treated with paclitaxel and cisplatin. In some embodiments, the subject has been previously treated with paclitaxel and carboplatin. In some embodiments, the subject has been previously treated with paclitaxel and topotecan. In some embodiments, the subject has been previously treated with bevacizumab, paclitaxel, and cisplatin. In some embodiments, the subject has been previously treated with bevacizumab, paclitaxel, and carboplatin. In some embodiments, the subject has been previously treated with bevacizumab, paclitaxel, and topotecan. In some embodiments, the subject was not previously treated with bevacizumab. In some embodiments, the subject is ineligible for treatment with bevacizumab. In some embodiments, the subject is not a candidate for curative therapy. In some embodiments, the curative therapy is radiotherapy and/or exenterative therapy. In some embodiments, the curative therapy is radiotherapy. In some embodiments, the curative therapy is exenterative therapy. In some embodiments, the subject is between 15 and 25 years old. In some embodiments, the subject is between 20 and 25 years old. In some embodiments, the subject is between 25 and 30 years old. In some embodiments, the subject is between 30 and 35 years old. In some embodiments, the subject is between 35 and 44 years old. In some embodiments, the subject is between 35 and 40 years old. In some embodiments, the subject is between 40 and 45 years old. In some embodiments, the subject is between 45 and 50 years old. In some embodiments, the subject is between 50 and 55 years old. In some embodiments, the subject is between 55 and 60 years old. In some embodiments, the subject is between 60 and 65 years old. In some embodiments, the subject is between 65 and 70 years old. In some embodiments, the subject is between 70 and 75 years old. In some embodiments, the subject is between 75 and 80 years old. In some embodiments, the subject is less than 65 years old. In some embodiments, the subject is greater than or equal to 65 years old. In some embodiments, the subject has a TF histology score of at least 1. The TF histology score is determined by analyzing membrane and cytoplasmic TF expression in a biopsy sample from the subject using an analytically validated immunohistochemistry assay. The TF histology-score (H-score) is calculated based on the percentage of tumor tissue that have membrane or cytoplasmic TF expression intensity of low (1+), intermediate (2+), and high (3+) on evaluable samples using the following equation: H-score=(1×[% cells 1+])+(2×[% cells 2+])+(3×[% cells 3+]). In some embodiments, the subject has an Eastern Cooperative Oncology Group (ECOG) score of 0. In some embodiments, the subject has an ECOG score of 1. In some embodiments, the subject has an ECOG score of 2. In some embodiments, the subject has an ECOG score of 3. In some embodiments, the subject has an ECOG score of 4. In a particular embodiment, the subject is a human.

In some embodiments of the methods or uses provided herein, the cervical cancer is an adenocarcinoma, an adenosquamous carcinoma, a squamous cell carcinoma, a small cell carcinoma, a neuroendocrine tumor, a glassy cell carcinoma or a villoglandular adenocarcinoma. In some embodiments, the cervical cancer is an adenocarcinoma, an adenosquamous carcinoma or a squamous cell carcinoma. In some embodiments, the cervical cancer is an adenocarcinoma. In some embodiments, the cervical cancer is an adenosquamous carcinoma. In some embodiments, the cervical cancer is a squamous cell carcinoma. In some embodiments, the cervical cancer is a non-squamous cell carcinoma. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

In some embodiments of the methods or uses provided herein, the cervical cancer is a stage 0, 1, 2, 3, or 4 cervical cancer. In some embodiments, the cervical cancer is a stage 0, 1A, 1B, 2A, 2B, 3A, 3B, 4A or 4B cervical cancer. In some embodiments, the cervical cancer is staged by the International Federation of Gynecology and Obstetrics (FIGO) staging system. In some embodiments, the staging is based on clinical examination. In some embodiments, in stage 0 cervical cancer the carcinoma is confined to the surface layer (cells lining) the cervix. In some embodiments, in stage 1 cervical cancer the carcinoma has grown deeper into the cervix but has not yet spread beyond it. In some embodiments, in stage 1A cervical cancer the invasive carcinoma can be diagnosed only by microscopy and the deepest invasion is less than 5 mm and the largest extension is less than 7 mm. In some embodiments, in stage 1B cervical cancer the lesions are clinically visible and are limited to the cervix uteri. In some embodiments, in stage 2 cervical cancer the cervical carcinoma has invaded beyond the uterus, but not to the pelvic wall or to the lower third of the vagina. In some embodiments, in stage 2A cervical cancer there is no parametrial invasion. In some embodiments, in stage 2B cervical cancer there is parametrial invasion. In some embodiments, in stage 3 cervical cancer the tumor extends to the pelvic wall and/or involves the lower third of the vagina and/or causes hydronephrosis or non-functioning kidney. In some embodiments, in stage 3A cervical cancer the tumor involves the lower third of the vagina, with no extension to the pelvic wall. In some embodiments, in stage 3B cervical cancer extends to the pelvic wall and/or cause hydronephrosis or non-functioning kidney. In some embodiments, in stage 4 cervical cancer, the carcinoma has extended beyond the true pelvis or has involved the mucosa of the bladder or rectum. In some embodiments, in stage 4A cervical cancer the tumor has spread to adjacent organs. In some embodiments, in stage 4B cervical cancer the tumor has spread to distant organs. In some embodiments, the cervical cancer is an advanced cervical cancer such as a grade 3 or grade 4 cervical cancer. In some embodiments, the advanced cervical cancer is metastatic cervical cancer. In some embodiments, the cervical cancer is metastatic cervical cancer and recurrent cervical cancer. In some embodiments, the cervical cancer is metastatic cervical cancer. In some embodiments, the cervical cancer is recurrent cervical cancer.

In some embodiments of the methods or uses provided herein, the subject has been previously treated for the cervical cancer. In some embodiments, the subject did not respond to the treatment (e.g., the subject experienced disease progression during treatment). In some embodiments, the one or more therapeutic agents administered to the subject was not an anti-TF antibody-drug conjugate as described herein. In some embodiments, the one or more therapeutic agents administered to the subject was paclitaxel, cisplatin, carboplatin, topotecan, gemcitabine, fluorouracil, ixabepilone, imatinib mesylate, docetaxel, gefitinib, nab-paclitaxel, pemetrexed, vinorelbine, doxil, cetuximab, pembrolizumab, nivolumab, bevacizumab, or any combination thereof. In some embodiments, the one or more therapeutic agents administered to the subject was gemcitabine. In some embodiments, the one or more therapeutic agents administered to the subject was fluorouracil. In some embodiments, the one or more therapeutic agents administered to the subject was ixabepilone. In some embodiments, the one or more therapeutic agents administered to the subject was imatinib mesylate. In some embodiments, the one or more therapeutic agents administered to the subject was docetaxel. In some embodiments, the one or more therapeutic agents administered to the subject was gefitinib. In some embodiments, the one or more therapeutic agents administered to the subject was nab-paclitaxel. In some embodiments, the one or more therapeutic agents administered to the subject was pemetrexed. In some embodiments, the one or more therapeutic agents administered to the subject was vinorelbine. In some embodiments, the one or more therapeutic agents administered to the subject was doxil. In some embodiments, the one or more therapeutic agents administered to the subject was cetuximab. In some embodiments, the one or more therapeutic agents administered to the subject was pembrolizumab. In some embodiments, the one or more therapeutic agents administered to the subject was nivolumab. In some embodiments, the one or more therapeutic agents administered to the subject was bevacizumab. In some embodiments, the one or more therapeutic agents administered to the subject was a platinum-based therapeutic agent. In some embodiments, the one or more therapeutic agents administered to the subject were gemcitabine and fluorouracil. In some embodiments, the one or more therapeutic agents administered to the subject were paclitaxel and cisplatin. In some embodiments, the one or more therapeutic agents administered to the subject were paclitaxel and carboplatin. In some embodiments, the one or more therapeutic agents administered to the subject were paclitaxel and topotecan. In some embodiments, the one or more therapeutic agents administered to the subject was bevacizumab. In some embodiments, the one or more therapeutic agents administered to the subject was selected from the group consisting of a chemotherapeutic agent, pemetrexed, nab-paclitaxel, vinorelbine, bevacizumab, cisplatin, carboplatin, paclitaxel, topotecan, a combination of bevacizumab and paclitaxel, a combination of bevacizumab and cisplatin, a combination of bevacizumab and carboplatin, a combination of paclitaxel and topotecan, a combination of bevacizumab and topotecan, a combination of bevacizumab, cisplatin and paclitaxel, a combination of bevacizumab, carboplatin and paclitaxel, and a combination of bevacizumab, paclitaxel and topotecan. In some embodiments, the one or more therapeutic agents administered to the subject was a chemotherapeutic agent. In some embodiments, the one or more therapeutic agents administered to the subject was cisplatin. In some embodiments, the one or more therapeutic agents administered to the subject was carboplatin. In some embodiments, the one or more therapeutic agents administered to the subject was paclitaxel. In some embodiments, the one or more therapeutic agents administered to the subject was topotecan. In some embodiments, the one or more therapeutic agents administered to the subject was a combination of bevacizumab and paclitaxel. In some embodiments, the one or more therapeutic agents administered to the subject was a combination of bevacizumab and cisplatin. In some embodiments, the one or more therapeutic agents administered to the subject was a combination of bevacizumab and carboplatin. In some embodiments, the one or more therapeutic agents administered to the subject was a combination of paclitaxel and topotecan. In some embodiments, the one or more therapeutic agents administered to the subject was a combination of bevacizumab and topotecan. In some embodiments, the one or more therapeutic agents administered to the subject was a combination of bevacizumab, cisplatin and paclitaxel. In some embodiments, the one or more therapeutic agents administered to the subject was a combination of bevacizumab, carboplatin and paclitaxel. In some embodiments, the one or more therapeutic agents administered to the subject was a combination of bevacizumab, paclitaxel and topotecan. In some embodiments, the subject received treatment for the cervical cancer with irradiation and did not respond to the irradiation. In some embodiments, the subject did not respond to treatment with no more than two prior systemic treatment regiments. In some embodiments, the subject did not respond to treatment with one or two prior systemic treatment regimens. In some embodiments, the subject did not respond to treatment with one prior systemic treatment regimen. In some embodiments, the subject did not respond to treatment with two prior systemic treatment regimens.

In some embodiments of the methods or uses provided herein, the subject has been previously treated for the cervical cancer with one or more therapeutic agents. In some embodiments, the subject relapsed after the treatment. In some embodiments, the one or more therapeutic agents administered to the subject was not an anti-TF antibody-drug conjugate as described herein. In some embodiments, the one or more therapeutic agents administered to the subject was paclitaxel, cisplatin, carboplatin, topotecan, gemcitabine, fluorouracil, ixabepilone, imatinib mesylate, docetaxel, gefitinib, nab-paclitaxel, pemetrexed, vinorelbine, doxil, cetuximab, pembrolizumab, nivolumab, bevacizumab, or any combination thereof. In some embodiments, the one or more therapeutic agents administered to the subject was gemcitabine. In some embodiments, the one or more therapeutic agents administered to the subject was fluorouracil. In some embodiments, the one or more therapeutic agents administered to the subject was ixabepilone. In some embodiments, the one or more therapeutic agents administered to the subject was imatinib mesylate. In some embodiments, the one or more therapeutic agents administered to the subject was docetaxel. In some embodiments, the one or more therapeutic agents administered to the subject was gefitinib. In some embodiments, the one or more therapeutic agents administered to the subject was nab-paclitaxel. In some embodiments, the one or more therapeutic agents administered to the subject was pemetrexed. In some embodiments, the one or more therapeutic agents administered to the subject was vinorelbine. In some embodiments, the one or more therapeutic agents administered to the subject was doxil. In some embodiments, the one or more therapeutic agents administered to the subject was cetuximab. In some embodiments, the one or more therapeutic agents administered to the subject was pembrolizumab. In some embodiments, the one or more therapeutic agents administered to the subject was nivolumab. In some embodiments, the one or more therapeutic agents administered to the subject was bevacizumab. In some embodiments, the one or more therapeutic agents administered to the subject was a platinum-based therapeutic agent. In some embodiments, the one or more therapeutic agents administered to the subject were gemcitabine and fluorouracil. In some embodiments, the one or more therapeutic agents administered to the subject were paclitaxel and cisplatin. In some embodiments, the one or more therapeutic agents administered to the subject were paclitaxel and carboplatin. In some embodiments, the one or more therapeutic agents administered to the subject were paclitaxel and topotecan. In some embodiments, the one or more therapeutic agents administered to the subject was bevacizumab. In some embodiments, the one or more therapeutic agents administered to the subject was selected from the group consisting of a chemotherapeutic agent, pemetrexed, nab-paclitaxel, vinorelbine, bevacizumab, cisplatin, carboplatin, paclitaxel, topotecan, a combination of bevacizumab and paclitaxel, a combination of bevacizumab and cisplatin, a combination of bevacizumab and carboplatin, a combination of paclitaxel and topotecan, a combination of bevacizumab and topotecan, a combination of bevacizumab, cisplatin and paclitaxel, a combination of bevacizumab, carboplatin and paclitaxel, and a combination of bevacizumab, paclitaxel and topotecan. In some embodiments, the one or more therapeutic agents administered to the subject was a chemotherapeutic agent. In some embodiments, the one or more therapeutic agents administered to the subject was cisplatin. In some embodiments, the one or more therapeutic agents administered to the subject was carboplatin. In some embodiments, the one or more therapeutic agents administered to the subject was paclitaxel. In some embodiments, the one or more therapeutic agents administered to the subject was topotecan. In some embodiments, the one or more therapeutic agents administered to the subject was a combination of bevacizumab and paclitaxel. In some embodiments, the one or more therapeutic agents administered to the subject was a combination of bevacizumab and cisplatin. In some embodiments, the one or more therapeutic agents administered to the subject was a combination of bevacizumab and carboplatin. In some embodiments, the one or more therapeutic agents administered to the subject was a combination of paclitaxel and topotecan. In some embodiments, the one or more therapeutic agents administered to the subject was a combination of bevacizumab and topotecan. In some embodiments, the one or more therapeutic agents administered to the subject was a combination of bevacizumab, cisplatin and paclitaxel. In some embodiments, the one or more therapeutic agents administered to the subject was a combination of bevacizumab, carboplatin and paclitaxel. In some embodiments, the one or more therapeutic agents administered to the subject was a combination of bevacizumab, paclitaxel and topotecan. In some embodiments, the subject received treatment for the cervical cancer with irradiation and relapsed after treatment with irradiation. In some embodiments, the subject relapsed after treatment with no more than two prior systemic treatment regiments. In some embodiments, the subject relapsed after treatment with one or two prior systemic treatment regimens. In some embodiments, the subject relapsed after treatment with one prior systemic treatment regimen. In some embodiments, the subject relapsed after treatment with two prior systemic treatment regimens.

In some embodiments of the methods or uses provided herein, the subject has been previously treated for the cervical cancer with one or more therapeutic agents. In some embodiments, the subject experienced disease progression after the treatment. In some embodiments, the one or more therapeutic agents administered to the subject was not an anti-TF antibody-drug conjugate as described herein. In some embodiments, the one or more therapeutic agents administered to the subject was paclitaxel, cisplatin, carboplatin, topotecan, gemcitabine, fluorouracil, ixabepilone, imatinib mesylate, docetaxel, gefitinib, nab-paclitaxel, pemetrexed, vinorelbine, doxil, cetuximab, pembrolizumab, nivolumab, bevacizumab, or any combination thereof. In some embodiments, the one or more therapeutic agents administered to the subject was gemcitabine. In some embodiments, the one or more therapeutic agents administered to the subject was fluorouracil. In some embodiments, the one or more therapeutic agents administered to the subject was ixabepilone. In some embodiments, the one or more therapeutic agents administered to the subject was imatinib mesylate. In some embodiments, the one or more therapeutic agents administered to the subject was docetaxel. In some embodiments, the one or more therapeutic agents administered to the subject was gefitinib. In some embodiments, the one or more therapeutic agents administered to the subject was nab-paclitaxel. In some embodiments, the one or more therapeutic agents administered to the subject was pemetrexed. In some embodiments, the one or more therapeutic agents administered to the subject was vinorelbine. In some embodiments, the one or more therapeutic agents administered to the subject was doxil. In some embodiments, the one or more therapeutic agents administered to the subject was cetuximab. In some embodiments, the one or more therapeutic agents administered to the subject was pembrolizumab. In some embodiments, the one or more therapeutic agents administered to the subject was nivolumab. In some embodiments, the one or more therapeutic agents administered to the subject was bevacizumab. In some embodiments, the one or more therapeutic agents administered to the subject was a platinum-based therapeutic agent. In some embodiments, the one or more therapeutic agents administered to the subject were gemcitabine and fluorouracil. In some embodiments, the one or more therapeutic agents administered to the subject were paclitaxel and cisplatin. In some embodiments, the one or more therapeutic agents administered to the subject were paclitaxel and carboplatin. In some embodiments, the one or more therapeutic agents administered to the subject were paclitaxel and topotecan. In some embodiments, the one or more therapeutic agents administered to the subject was bevacizumab. In some embodiments, the one or more therapeutic agents administered to the subject was selected from the group consisting of a chemotherapeutic agent, pemetrexed, nab-paclitaxel, vinorelbine, bevacizumab, cisplatin, carboplatin, paclitaxel, topotecan, a combination of bevacizumab and paclitaxel, a combination of bevacizumab and cisplatin, a combination of bevacizumab and carboplatin, a combination of paclitaxel and topotecan, a combination of bevacizumab and topotecan, a combination of bevacizumab, cisplatin and paclitaxel, a combination of bevacizumab, carboplatin and paclitaxel, and a combination of bevacizumab, paclitaxel and topotecan. In some embodiments, the one or more therapeutic agents administered to the subject was a chemotherapeutic agent. In some embodiments, the one or more therapeutic agents administered to the subject was cisplatin. In some embodiments, the one or more therapeutic agents administered to the subject was carboplatin. In some embodiments, the one or more therapeutic agents administered to the subject was paclitaxel. In some embodiments, the one or more therapeutic agents administered to the subject was topotecan. In some embodiments, the one or more therapeutic agents administered to the subject was a combination of bevacizumab and paclitaxel. In some embodiments, the one or more therapeutic agents administered to the subject was a combination of bevacizumab and cisplatin. In some embodiments, the one or more therapeutic agents administered to the subject was a combination of bevacizumab and carboplatin. In some embodiments, the one or more therapeutic agents administered to the subject was a combination of paclitaxel and topotecan. In some embodiments, the one or more therapeutic agents administered to the subject was a combination of bevacizumab and topotecan. In some embodiments, the one or more therapeutic agents administered to the subject was a combination of bevacizumab, cisplatin and paclitaxel. In some embodiments, the one or more therapeutic agents administered to the subject was a combination of bevacizumab, carboplatin and paclitaxel. In some embodiments, the one or more therapeutic agents administered to the subject was a combination of bevacizumab, paclitaxel and topotecan. In some embodiments, the subject previously received treatment for the cervical cancer with irradiation and experienced disease progression after treatment with irradiation. In some embodiments, the subject experienced disease progression after treatment with no more than two prior systemic treatment regiments. In some embodiments, the subject experienced disease progression after treatment with one or two prior systemic treatment regimens. In some embodiments, the subject experienced disease progression after treatment with one prior systemic treatment regimen. In some embodiments, the subject experienced disease progression after treatment with two prior systemic treatment regimens.

In some embodiments, eligible subjects had second- or third-line recurrent or metastatic cervical cancer and had experienced disease progression on or after receiving a chemotherapy doublet (paclitaxel+cisplatin/carboplatin OR paclitaxel+topotecan) in combination with bevacizumab, if eligible to receive bevacizumab according to local standards. Prior chemoradiation was not considered a line of therapy. Bevacizumab in combination with cisplatin/carboplatin+paclitaxel OR paclitaxel+topotecan is hereafter referred to as “bevacizumab+chemotherapy doublet”.

B. Routes of Administration

An antibody-drug conjugate or antigen-binding fragment thereof described herein can be administered by any suitable route and mode. Suitable routes of administering antibody-drug conjugate of the present invention are well known in the art and may be selected by those of ordinary skill in the art. In one embodiment, the antibody-drug conjugate is administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an antibody-drug conjugate or antigen-binding fragment described herein is intravenous injection or infusion. In some embodiments, the route of administration of an antibody-drug conjugate or antigen-binding fragment described herein is intravenous infusion.

C. Dosage and Frequency of Administration

In one aspect, the present invention provides for methods of treating a subject with cervical cancer as described herein with a particular dose of an antibody-drug conjugate or antigen-binding fragment thereof as described herein, wherein the subject is administered the antibody-drug conjugate or antigen-binding fragment thereof as described herein with a particular frequency until disease progression or unacceptable toxicity.

In one embodiment of the methods or uses provided herein, an antibody-drug conjugate or antigen-binding fragment thereof as described herein is administered to the subject at a dose ranging from about 1.5 mg/kg to about 2.1 mg/kg of the subject's body weight. In certain embodiments, the dose is about 1.5 mg/kg, about 1.6 mg/kg, about 1.7 mg/kg, about 1.8 mg/kg, about 1.9 mg/kg, about 2.0 mg/kg or about 2.1 mg/kg. In one embodiment, the dose is about 2.0 mg/kg. In one embodiment, the dose is 2.0 mg/kg. In some embodiments, the dose is 2.0 mg/kg and the antibody-drug conjugate is tisotumab vedotin.

In one embodiment of the methods or uses or product for uses provided herein, an anti-TF antibody-drug conjugate or antigen-binding fragment thereof as described herein is administered to the subject at a dose ranging from about 0.65 mg/kg to about 2.1 mg/kg of the subject's body weight. In certain embodiments, the dose is about 0.65 mg/kg, about 0.7 mg/kg, about 0.75 mg/kg, about 0.8 mg/kg, about 0.85 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg or about 2.1 mg/kg. In one embodiment, the dose is about 0.65 mg/kg. In one embodiment, the dose is about 0.9 mg/kg. In one embodiment, the dose is about 1.3 mg/kg. In one embodiment, the dose is about 2.0 mg/kg. In certain embodiments, the dose is 0.65 mg/kg, 0.7 mg/kg, 0.75 mg/kg, 0.8 mg/kg, 0.85 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg or 2.1 mg/kg. In one embodiment, the dose is 0.65 mg/kg. In one embodiment, the dose is 0.9 mg/kg. In one embodiment, the dose is 1.3 mg/kg. In one embodiment, the dose is 2.0 mg/kg. In some embodiments, the dose is 0.65 mg/kg and the anti-TF antibody-drug conjugate is tisotumab vedotin. In some embodiments, the dose is 0.9 mg/kg and the anti-TF antibody-drug conjugate is tisotumab vedotin. In some embodiments, the dose is 1.3 mg/kg and the anti-TF antibody-drug conjugate is tisotumab vedotin. In some embodiments, the dose is 2.0 mg/kg and the anti-TF antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, 130 mg, or 200 mg.

In one embodiment of the methods or uses provided herein, an antibody-drug conjugate or antigen-binding fragment thereof as described herein is administered to the subject once about every 1 to 4 weeks. In certain embodiments, the an antibody-drug conjugate or antigen-binding fragment thereof as described herein is administered once about every 1 week, once about every 2 weeks, once about every 3 weeks or once about every 4 weeks. In one embodiment, an antibody-drug conjugate or antigen-binding fragment thereof as described herein is administered once about every 3 weeks. In one embodiment, an antibody-drug conjugate or antigen-binding fragment thereof as described herein is administered once every 3 weeks. In some embodiments, the dose is about 0.65 mg/kg and is administered once about every 1 week. In some embodiments, the dose is about 0.65 mg/kg and is administered once about every 2 weeks. In some embodiments, the dose is about 0.65 mg/kg and is administered once about every 3 weeks. In some embodiments, the dose is about 0.65 mg/kg and is administered once about every 4 weeks. In some embodiments, the dose is about 0.7 mg/kg and is administered once about every 1 week. In some embodiments, the dose is about 0.7 mg/kg and is administered once about every 2 weeks. In some embodiments, the dose is about 0.7 mg/kg and is administered once about every 3 weeks. In some embodiments, the dose is about 0.7 mg/kg and is administered once about every 4 weeks. In some embodiments, the dose is about 0.75 mg/kg and is administered once about every 1 week. In some embodiments, the dose is about 0.75 mg/kg and is administered once about every 2 weeks. In some embodiments, the dose is about 0.75 mg/kg and is administered once about every 3 weeks. In some embodiments, the dose is about 0.75 mg/kg and is administered once about every 4 weeks. In some embodiments, the dose is about 0.8 mg/kg and is administered once about every 1 week. In some embodiments, the dose is about 0.8 mg/kg and is administered once about every 2 weeks. In some embodiments, the dose is about 0.8 mg/kg and is administered once about every 3 weeks. In some embodiments, the dose is about 0.8 mg/kg and is administered once about every 4 weeks. In some embodiments, the dose is about 0.85 mg/kg and is administered once about every 1 week. In some embodiments, the dose is about 0.85 mg/kg and is administered once about every 2 weeks. In some embodiments, the dose is about 0.85 mg/kg and is administered once about every 3 weeks. In some embodiments, the dose is about 0.85 mg/kg and is administered once about every 4 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is 0.65 mg/kg and is administered once about every 1 week. In some embodiments, the dose is 0.65 mg/kg and is administered once about every 2 weeks. In some embodiments, the dose is 0.65 mg/kg and is administered once about every 3 weeks. In some embodiments, the dose is 0.65 mg/kg and is administered once about every 4 weeks. In some embodiments, the dose is 0.7 mg/kg and is administered once about every 1 week. In some embodiments, the dose is 0.7 mg/kg and is administered once about every 2 weeks. In some embodiments, the dose is 0.7 mg/kg and is administered once about every 3 weeks. In some embodiments, the dose is 0.7 mg/kg and is administered once about every 4 weeks. In some embodiments, the dose is 0.75 mg/kg and is administered once about every 1 week. In some embodiments, the dose is 0.75 mg/kg and is administered once about every 2 weeks. In some embodiments, the dose is 0.75 mg/kg and is administered once about every 3 weeks. In some embodiments, the dose is 0.75 mg/kg and is administered once about every 4 weeks. In some embodiments, the dose is 0.8 mg/kg and is administered once about every 1 week. In some embodiments, the dose is 0.8 mg/kg and is administered once about every 2 weeks. In some embodiments, the dose is 0.8 mg/kg and is administered once about every 3 weeks. In some embodiments, the dose is 0.8 mg/kg and is administered once about every 4 weeks. In some embodiments, the dose is 0.85 mg/kg and is administered once about every 1 week. In some embodiments, the dose is 0.85 mg/kg and is administered once about every 2 weeks. In some embodiments, the dose is 0.85 mg/kg and is administered once about every 3 weeks. In some embodiments, the dose is 0.85 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.0 mg/kg and is administered once about every 3 weeks (e.g., ±3 days). In some embodiments, the dose is 2.0 mg/kg and is administered once every 3 weeks. In some embodiments, the dose is 2.0 mg/kg and is administered once every 3 weeks and the antibody-drug conjugate is tisotumab vedotin. In some embodiments, the dose is 2.0 mg/kg and is administered once every 3 weeks and the antibody-drug conjugate is tisotumab vedotin and the dose is decreased to 1.3 mg/kg if one or more adverse events occur. In some embodiments, the dose is 2.0 mg/kg and is administered once every 3 weeks and the antibody-drug conjugate is tisotumab vedotin and the dose is decreased to 1.3 mg/kg if one or more adverse events occur and the one or more adverse events is an ocular adverse event. In some embodiments, the dose is 2.0 mg/kg and is administered once every 3 weeks and the antibody-drug conjugate is tisotumab vedotin and the dose is decreased to 1.3 mg/kg if one or more adverse events occur and the one or more adverse events is peripheral neuropathy. In some embodiments, the dose is 1.3 mg/kg and is administered once every 3 weeks. In some embodiments, the dose is 1.3 mg/kg and is administered once every 3 weeks and the antibody-drug conjugate is tisotumab vedotin. In some embodiments, the dose is 1.3 mg/kg and is administered once every 3 weeks and the antibody-drug conjugate is tisotumab vedotin and the dose is decreased to 0.9 mg/kg if one or more adverse events occur. In some embodiments, the dose is 1.3 mg/kg and is administered once every 3 weeks and the antibody-drug conjugate is tisotumab vedotin and the dose is decreased to 0.9 mg/kg if one or more adverse events occur and the one or more adverse events is an ocular adverse event. In some embodiments, the dose is 1.3 mg/kg and is administered once every 3 weeks and the antibody-drug conjugate is tisotumab vedotin and the dose is decreased to 0.9 mg/kg if one or more adverse events occur and the one or more adverse events is peripheral neuropathy. In some embodiments, the dose is about 0.9 mg/kg and is administered once about every week and the antibody-drug conjugate is tisotumab vedotin. In some embodiments, the dose is 0.9 mg/kg and is administered once every week and the antibody-drug conjugate is tisotumab vedotin. In some embodiments, the dose is about 0.65 mg/kg and is administered once about every week and the antibody-drug conjugate is tisotumab vedotin. In some embodiments, the dose is 0.65 mg/kg and is administered once every week and the antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, 130 mg, or 200 mg.

In one embodiment of the methods or uses provided herein, an antibody-drug conjugate or antigen-binding fragment thereof as described herein is administered to the subject at a flat dose of between about 50 mg and about 200 mg such as at a dose of about 50 mg or a dose of about 60 mg or a dose of about 70 mg or a dose of about 80 mg or a dose of about 90 mg or a dose of about 100 mg or a dose of about 110 mg or a dose of about 120 mg or a dose of about 130 mg or a dose of about 140 mg or a dose of about 150 mg or a dose of about 160 mg or a dose of about 170 mg or a dose of about 180 mg or a dose of about 190 mg or a dose of about 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., 3 days). In some embodiments, the flat dose is administered to the subject once every 3 weeks. In some embodiments, the flat dose is administered to the subject once every 3 weeks and the antibody-drug conjugate is tisotumab vedotin.

In one embodiment of the methods or uses provided herein, an antibody-drug conjugate or antigen-binding fragment thereof as described herein is administered to the subject at a flat dose of between 50 mg and 200 mg such as at a dose of 50 mg or a dose of 60 mg or a dose of 70 mg or a dose of 80 mg or a dose of 90 mg or a dose of 100 mg or a dose of 110 mg or a dose of 120 mg or a dose of 130 mg or a dose of 140 mg or a dose of 150 mg or a dose of 160 mg or a dose of 170 mg or a dose of 180 mg or a dose of 190 mg or a dose of 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., ±3 days). In some embodiments, the flat dose is administered to the subject once every 3 weeks. In some embodiments, the flat dose is administered to the subject once every 3 weeks and the antibody-drug conjugate is tisotumab vedotin.

In some embodiments, a method of treatment or use described herein further comprises the administration of one or more additional therapeutic agents. In some embodiments, the one or more additional therapeutic agents are administered simultaneously with an antibody-drug conjugate or antigen-binding fragment thereof as described herein, such as tisotumab vedotin. In some embodiments, the one or more additional therapeutic agents and an antibody-drug conjugate or antigen-binding fragment thereof as described herein are administered sequentially.

D. Treatment Outcome

In one aspect, a method of treating cervical cancer with an antibody-drug conjugates or antigen-binding fragments thereof described herein results in an improvement in one or more therapeutic effects in the subject after administration of the antibody-drug conjugate relative to a baseline. In some embodiments, the one or more therapeutic effects is the size of the tumor derived from the cervical cancer, the objective response rate, the duration of response, the time to response, progression free survival, overall survival, or any combination thereof. In one embodiment, the one or more therapeutic effects is the size of the tumor derived from the cervical cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses provided herein, response to treatment with an antibody-drug conjugate or antigen-binding fragment thereof described herein may include the following criteria (RECIST Criteria 1.1):

| | Category | Criteria |
|---|---|---|
| Based on | Complete | Disappearance of all target lesions. Any |
| target | Response | pathological lymph nodes must have reduction |
| lesions | (CR) | in short axis to <10 mm. |
| | Partial | ≥30% decrease in the sum of the longest |
| | Response | diameter (LD) of target lesions, taking as |
| | (PR) | reference the baseline sum of LDs. |
| | Stable | Neither sufficient shrinkage to qualify for |
| | Disease | PR nor sufficient increase to qualify for PD, |
| | (SD) | taking as reference the smallest sum of LDs |
| | | while in trial. |
| | Progressive | ≥20% (and ≥5 mm) increase in the sum of the |
| | Disease | LDs of target lesions, taking as reference the |
| | (PD) | smallest sum of the target LDs recorded while |
| | | in trial or the appearance of one or more |
| | | new lesions. |
| Based on | CR | Disappearance of all non-target lesions and |
| non-target | | normalization of tumor marker level. All lymph |
| lesions | | nodes must be non-pathological in size (<10 |
| | | mm short axis). |
| | SD | Persistence of one or more non-target lesion(s) |
| | | or/and maintenance of tumor marker level |
| | | above the normal limits. |
| | PD | Appearance of one or more new lesions and/or |
| | | unequivocal progression of existing non-target |
| | | lesions. |

In one embodiment of the methods or uses provided herein, the effectiveness of treatment with an antibody-drug conjugate or antigen-binding fragment thereof described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 300%-80%. In one embodiment, the objective response rate is at least about 400%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is 100%. In one embodiment, the objective response rate is between about 13% and about 35%. In one embodiment, the objective response rate is at least about 14%. In one embodiment, the objective response rate is at least about 19%. In one embodiment, the objective response rate is at least about 21%. In one embodiment, the objective response rate is at least about 23.8%. In one embodiment, the objective response rate is 23.8%. In one embodiment, the objective response rate is at least about 24%. In one embodiment, the objective response rate is at least about 25%. In one embodiment, the objective response rate is at least about 26%. In one embodiment, the objective response rate is at least about 28%. In one embodiment, the objective response rate is at least about 30%. In one embodiment, the objective response rate is at least about 33%. In some embodiments, the objective response rate is between about 13% and about 35% and the patient received prior radiation to the pelvis. In some embodiments, the objective response rate is between about 13% and about 35%, the patient received prior radiation to the pelvis, and the patient experienced disease progression after the prior radiation to the pelvis. In some embodiments, the objective response rate is between about 13% and about 35% and the patient has not received prior radiation to the pelvis. In some embodiments, the objective response rate is at least about 28.2% and the patient received 1 prior systemic treatment regimen. In some embodiments, the objective response rate is at least about 13.3% and the patient received 2 prior systemic treatment regimens. In some embodiments, the objective response rate is at least about 28.2%, the patient received 1 prior systemic treatment regimen, and the patient experienced disease progression after the prior systemic treatment regimen. In some embodiments, the objective response rate is at least about 13.3%, the patient received 2 prior systemic treatment regimens, and the patient experienced disease progression after the 2 prior systemic treatment regimens. In some embodiments, the prior systemic therapy was bevacizumab. In some embodiments, the prior systemic therapy was a chemotherapy. In some embodiments, the prior systemic therapy was a combination of a chemotherapy and bevacizumab. In some embodiments, the prior systemic therapy was a combination of doublet chemotherapy and bevacizumab. In some embodiments the doublet chemotherapy is a combination of paclitaxel and cisplatin. In some embodiments the doublet chemotherapy is a combination of paclitaxel and carboplatin. In some embodiments, the doublet chemotherapy is a combination of paclitaxel and topotecan. In some embodiments, the prior systemic therapy was a checkpoint inhibitor. In some embodiments, the prior systemic therapy was a pembrolizumab. In some embodiments, the objective response rate is at least about 25.5% and the subject was previously treated with cisplatin and radiation. In some embodiments, the objective response rate is at least about 21.7% and the subject was not previously treated with cisplatin and radiation. In some embodiments, the objective response rate is at least about 18.8% and the subject was previously treated with bevacizumab in combination with a chemotherapy doublet. In some embodiments, the objective response rate is at least about 32.4% and the subject was not previously treated with bevacizumab in combination with a chemotherapy doublet. In some embodiments, the objective response rate is at least about 26.3 and the cervical cancer responded to the last prior systemic therapy regimen. In some embodiments, the objective response rate is at least about 21.1% and the cervical cancer did not respond to the last prior systemic therapy regimen. In some embodiments, the objective response rate is at least about 23.2% and the cervical cancer is a squamous cell carcinoma. In some embodiments, the objective response rate is at last about 25.0% and the cervical cancer has non-squamous histology. In some embodiments, the objective response rate is at least about 25.0% and the cervical cancer is an adenocarcinoma. In some embodiments, the objective response rate is at least about 25.0% and the cervical cancer is an adenosquamous carcinoma. In some embodiments, the objective response rate is at least about 30.5% and the subject has an Eastern Cooperative Oncology Group (ECOG) score of 0. See e.g., Oken M, Creech R, Tormey D, et al. Toxicity and response criteria of the Eastern Cooperative Oncology Group. Am J Clin Oncol. 1982; 5:649-655. In some embodiments, the objective response rate is at least about 14.3% and the subject has an ECOG score of 1. In some embodiments, the objective response rate is between about 13% and about 35% and the subject has an ECOG score of 2. In some embodiments, the objective response rate is between about 13% and about 35% and the subject has an ECOG score of 3. In some embodiments, the objective response rate is between about 13% and about 35% and the subject has an ECOG score of 4. In some embodiments, the objective response rate is between about 13% and about 35% and the cervical cancer cells are positive for membrane TF expression. In some embodiments, the objective response rate is between about 13% and about 35% and the cervical cancer cells are positive for cytoplasmic TF expression. In some embodiments, positive TF expression is defined as ≥1% of cervical cancer cells expressing TF. In some embodiments, the objective response rate is between about 13% and about 35% and the subject has a TF histology score (H-score) of at least 1. In some embodiments, the objective response rate is at least about 27.3% and the subject is less than 65 years old. In some embodiments, the objective response rate is between about 13% and about 35% and the subject is greater than or equal to 65 years old. In some embodiments, the objective response rate is between about 13% and about 35% and the cervical cancer is a stage 3 cervical cancer. In some embodiments, the objective response rate is between about 13% and about 35% and the cervical cancer is a stage 4 cervical cancer.

In one embodiment of the methods or uses provided herein, response to treatment with an antibody-drug conjugate or antigen-binding fragment thereof described herein is assessed by measuring the size of a tumor derived from the cervical cancer. In one embodiment, the size of a tumor derived from the cervical cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate. In one embodiment, the size of a tumor derived from the cervical cancer is reduced by at least about 10%-80%. In one embodiment, the size of a tumor derived from the cervical cancer is reduced by at least about 20%-80%. In one embodiment, the size of a tumor derived from the cervical cancer is reduced by at least about 30%-80%. In one embodiment, the size of a tumor derived from the cervical cancer is reduced by at least about 40%-80%. In one embodiment, the size of a tumor derived from the cervical cancer is reduced by at least about 50%-80%. In one embodiment, the size of a tumor derived from the cervical cancer is reduced by at least about 60%-80%. In one embodiment, the size of a tumor derived from the cervical cancer is reduced by at least about 70%-80%. In one embodiment, the size of a tumor derived from the cervical cancer is reduced by at least about 80%. In one embodiment, the size of a tumor derived from the cervical cancer is reduced by at least about 85%. In one embodiment, the size of a tumor derived from the cervical cancer is reduced by at least about 90%. In one embodiment, the size of a tumor derived from the cervical cancer is reduced by at least about 95%. In one embodiment, the size of a tumor derived from the cervical cancer is reduced by at least about 98%. In one embodiment, the size of a tumor derived from the cervical cancer is reduced by at least about 99%. In one embodiment, the size of a tumor derived from the cervical cancer is reduced by 100%. In one embodiment, the size of a tumor derived from the cervical cancer is reduced by at least about 30%. In one embodiment, the size of a tumor derived from the cervical cancer is measured by magnetic resonance imaging (MRI). In one embodiment, the size of a tumor derived from the cervical cancer is measured by computed tomography (CT). In some embodiments, the size of a tumor derived from the cervical cancer is measured by pelvic examination. See Choi et al., 2008, J. Gynecol. Oncol. 19(3):205.

In one embodiment of the methods or uses provided described herein, response to treatment with an antibody-drug conjugate or antigen-binding fragment thereof described herein, such as e.g., tisotumab vedotin, promotes regression of a tumor derived from the cervical cancer. In one embodiment, a tumor derived from the cervical cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate. In one embodiment, a tumor derived from the cervical cancer regresses by at least about 10%-80%. In one embodiment, a tumor derived from the cervical cancer regresses by at least about 20%-80%. In one embodiment, a tumor derived from the cervical cancer regresses by at least about 30%-80%. In one embodiment, a tumor derived from the cervical cancer regresses by at least about 40%-80%. In one embodiment, a tumor derived from the cervical cancer regresses by at least about 50%-80%. In one embodiment, a tumor derived from the cervical cancer regresses by at least about 60%-80%. In one embodiment, a tumor derived from the cervical cancer regresses by at least about 70%-80%. In one embodiment, a tumor derived from the cervical cancer regresses by at least about 80%. In one embodiment, a tumor derived from the cervical cancer regresses by at least about 85%. In one embodiment, a tumor derived from the cervical cancer regresses by at least about 90%. In one embodiment, a tumor derived from the cervical cancer regresses by at least about 95%. In one embodiment, a tumor derived from the cervical cancer regresses by at least about 98%. In one embodiment, a tumor derived from the cervical cancer regresses by at least about 99%. In one embodiment, a tumor derived from the cervical cancer regresses by 100%. In one embodiment, a tumor derived from the cervical cancer regresses by at least about 30%. In one embodiment, regression of a tumor is determined by measuring the size of the tumor by magnetic resonance imaging (MRI). In one embodiment, regression of a tumor is determined by measuring the size of the tumor by computed tomography (CT). In some embodiments, regression of a tumor is determined by measuring the size of the tumor by pelvic examination. See Choi et al., 2008, J. Gynecol. Oncol. 19(3):205.

In one embodiment of the methods or uses described herein, response to treatment with an antibody-drug conjugate or antigen-binding fragment thereof described herein is assessed by measuring the time of progression free survival after administration of the antibody-drug conjugate. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the subject exhibits progression-free survival of at least about 6 months after administration of the antibody-drug conjugate. In some embodiments, the subject exhibits progression-free survival of at least about one year after administration of the antibody-drug conjugate. In some embodiments, the subject exhibits progression-free survival of at least about two years after administration of the antibody-drug conjugate. In some embodiments, the subject exhibits progression-free survival of at least about three years after administration of the antibody-drug conjugate. In some embodiments, the subject exhibits progression-free survival of at least about four years after administration of the antibody-drug conjugate. In some embodiments, the subject exhibits progression-free survival of at least about five years after administration of the antibody-drug conjugate. In some embodiments, the subject exhibits progression-free survival for at least about 3 months after administration of the antibody-drug conjugate. In some embodiments, the subject exhibits progression-free survival for at least about 4 months after administration of the antibody-drug conjugate. In some embodiments, the subject exhibits progression-free survival of at least about 4.2 months after administration of the antibody-drug conjugate. In some embodiments, the subject exhibits progression-free survival of at least 4.2 months after administration of the antibody-drug conjugate. In some embodiments, the subject exhibits progression-free survival for at least about 5 months after administration of the antibody-drug conjugate. In some embodiments, the subject exhibits progression-free survival of at least about 3 months after administration of the antibody-drug conjugate and the patient received prior radiation to the pelvis. In some embodiments, the subject exhibits progression-free survival of at least about 3 months after administration of the antibody-drug conjugate, the patient received prior radiation to the pelvis, and the patient experienced disease progression after the prior radiation to the pelvis. In some embodiments, the subject exhibits progression-free survival of at least about 3 months after administration of the antibody-drug conjugate and the patient has not received prior radiation to the pelvis. In some embodiments, the subject exhibits progression-free survival of at least about 3 months after administration of the antibody-drug conjugate and the patient received 1 prior systemic treatment regimen. In some embodiments, the subject exhibits progression-free survival of at least about 3 months after administration of the antibody-drug conjugate and the patient received 2 prior systemic treatment regimens. In some embodiments, the subject exhibits progression-free survival of at least about 3 months after administration of the antibody-drug conjugate, the patient received 1 prior systemic treatment regimen, and the patient experienced disease progression after the prior systemic treatment regimen. In some embodiments, the subject exhibits progression-free survival of at least about 3 months after administration of the antibody-drug conjugate, the patient received 2 prior systemic treatment regimens, and the patient experienced disease progression after the 2 prior systemic treatment regimens. In some embodiments, the prior systemic therapy was bevacizumab. In some embodiments, the prior systemic therapy was a chemotherapy. In some embodiments, the prior systemic therapy was a combination of a chemotherapy and bevacizumab. In some embodiments, the prior systemic therapy was a combination of doublet chemotherapy and bevacizumab. In some embodiments the doublet chemotherapy is a combination of paclitaxel and cisplatin. In some embodiments the doublet chemotherapy is a combination of paclitaxel and carboplatin. In some embodiments, the doublet chemotherapy is a combination of paclitaxel and topotecan. In some embodiments, the prior systemic therapy was a checkpoint inhibitor. In some embodiments, the prior systemic therapy was a pembrolizumab. In some embodiments, the subject exhibits progression-free survival of at least about 3 months after administration of the antibody-drug conjugate and the cervical cancer is a squamous cell carcinoma. In some embodiments, the subject exhibits progression-free survival of at least about 3 months after administration of the antibody-drug conjugate and the cervical cancer is an adenocarcinoma. In some embodiments, the subject exhibits progression-free survival of at least about 3 months after administration of the antibody-drug conjugate and the cervical cancer is an adenosquamous carcinoma. In some embodiments, the subject exhibits progression-free survival of at least about 3 months after administration of the antibody-drug conjugate and the subject has an ECOG score of 0. In some embodiments, the subject exhibits progression-free survival of at least about 3 months after administration of the antibody-drug conjugate and the subject has an ECOG score of 1. In some embodiments, the subject exhibits progression-free survival of at least about 3 months after administration of the antibody-drug conjugate and the subject has an ECOG score of 2. In some embodiments, the subject exhibits progression-free survival of at least about 3 months after administration of the antibody-drug conjugate and the subject has an ECOG score of 3. In some embodiments, the subject exhibits progression-free survival of at least about 3 months after administration of the antibody-drug conjugate and the subject has an ECOG score of 4. In some embodiments, the subject exhibits progression-free survival of at least about 3 months after administration of the antibody-drug conjugate and the cervical cancer cells are positive for membrane TF expression. In some embodiments, the subject exhibits progression-free survival of at least about 3 months after administration of the antibody-drug conjugate and the cervical cancer cells are positive for cytoplasmic TF expression. In some embodiments, positive TF expression is defined as ≥1% of cervical cancer cells expressing TF. In some embodiments, the subject exhibits progression-free survival of at least about 3 months after administration of the antibody-drug conjugate and the subject has a TF histology score (H-score) of at least 1. In some embodiments, the subject exhibits progression-free survival of at least about 3 months after administration of the antibody-drug conjugate and the subject is less than 65 years old. In some embodiments, the subject exhibits progression-free survival of at least about 3 months after administration of the antibody-drug conjugate and the subject is greater than or equal to 65 years old. In some embodiments, the subject exhibits progression-free survival of at least about 3 months after administration of the antibody-drug conjugate and the cervical cancer is a stage 3 cervical cancer. In some embodiments, the subject exhibits progression-free survival of at least about 3 months after administration of the antibody-drug conjugate and the cervical cancer is a stage 4 cervical cancer.

In one embodiment of the methods or uses described herein, response to treatment with an antibody-drug conjugate or antigen-binding fragment thereof described herein is assessed by measuring the time to response to administration of the antibody-drug conjugate. In some embodiments, the time to response is less than about 1 month, about 2 months, about 3 months, about 4 months, about 5 months, about 6 months, about 7 months, about 8 months, about 9 months, about 10 months, about 11 months, or about 12 months after administration of the antibody-drug conjugate. In some embodiments, the time to response is less than about 1 month after administration of the antibody-drug conjugate. In some embodiments, the time to response is less than about 1.2 months after administration of the antibody-drug conjugate. In some embodiments, the time to response is less than about 1.4 months after administration of the antibody-drug conjugate. In some embodiments, the time to response is less than about 2 months after administration of the antibody-drug conjugate. In some embodiments, the time to response is less than about 3 months after administration of the antibody-drug conjugate. In some embodiments, the time to response is less than about 4 months after administration of the antibody-drug conjugate. In some embodiments, the time to response is less than about 5 months after administration of the antibody-drug conjugate. In some embodiments, the time to response is less than about 6 months after administration of the antibody-drug conjugate. In some embodiments, the time to response is less than about 6 months after administration of the antibody-drug conjugate. In some embodiments, the time to response is less than about 6 months after administration of the antibody-drug conjugate and the patient received prior radiation to the pelvis. In some embodiments, the time to response is less than about 6 months after administration of the antibody-drug conjugate, the patient received prior radiation to the pelvis, and the patient experienced disease progression after the prior radiation to the pelvis. In some embodiments, the time to response is less than about 6 months after administration of the antibody-drug conjugate and the patient has not received prior radiation to the pelvis. In some embodiments, the time to response is less than about 6 months after administration of the antibody-drug conjugate and the patient received 1 prior systemic treatment regimen. In some embodiments, the time to response is less than about 6 months after administration of the antibody-drug conjugate and the patient received 2 prior systemic treatment regimens. In some embodiments, the time to response is less than about 6 months after administration of the antibody-drug conjugate, the patient received 1 prior systemic treatment regimen, and the patient experienced disease progression after the prior systemic treatment regimen. In some embodiments, the time to response is less than about 6 months after administration of the antibody-drug conjugate, the patient received 2 prior systemic treatment regimens, and the patient experienced disease progression after the 2 prior systemic treatment regimens. In some embodiments, the prior systemic therapy was bevacizumab. In some embodiments, the prior systemic therapy was a chemotherapy. In some embodiments, the prior systemic therapy was a combination of a chemotherapy and bevacizumab. In some embodiments, the prior systemic therapy was a combination of doublet chemotherapy and bevacizumab. In some embodiments the doublet chemotherapy is a combination of paclitaxel and cisplatin. In some embodiments the doublet chemotherapy is a combination of paclitaxel and carboplatin. In some embodiments, the doublet chemotherapy is a combination of paclitaxel and topotecan. In some embodiments, the prior systemic therapy was a checkpoint inhibitor. In some embodiments, the prior systemic therapy was a pembrolizumab. In some embodiments, the time to response is less than about 6 months after administration of the antibody-drug conjugate and the cervical cancer is a squamous cell carcinoma. In some embodiments, the time to response is less than about 6 months after administration of the antibody-drug conjugate and the cervical cancer is an adenocarcinoma. In some embodiments, the time to response is less than about 6 months after administration of the antibody-drug conjugate and the cervical cancer is an adenosquamous carcinoma. In some embodiments, the time to response is less than about 6 months after administration of the antibody-drug conjugate and the subject has an ECOG score of 0. In some embodiments, the time to response is less than about 6 months after administration of the antibody-drug conjugate and the subject has an ECOG score of 1. In some embodiments, the time to response is less than about 6 months after administration of the antibody-drug conjugate and the subject has an ECOG score of 2. In some embodiments, the time to response is less than about 6 months after administration of the antibody-drug conjugate and the subject has an ECOG score of 3. In some embodiments, the time to response is less than about 6 months after administration of the antibody-drug conjugate and the subject has an ECOG score of 4. In some embodiments, the time to response is less than about 6 months after administration of the antibody-drug conjugate and the cervical cancer cells are positive for membrane TF expression. In some embodiments, the time to response is less than about 6 months after administration of the antibody-drug conjugate and the cervical cancer cells are positive for cytoplasmic TF expression. In some embodiments, positive TF expression is defined as ≥1% of cervical cancer cells expressing TF. In some embodiments, the time to response is less than about 6 months after administration of the antibody-drug conjugate and the subject has a TF histology score (H-score) of at least 1. In some embodiments, the time to response is less than about 6 months after administration of the antibody-drug conjugate and the subject is less than 65 years old. In some embodiments, the time to response is less than about 6 months after administration of the antibody-drug conjugate and the subject is greater than or equal to 65 years old. In some embodiments, the time to response is less than about 6 months after administration of the antibody-drug conjugate and the cervical cancer is a stage 3 cervical cancer. In some embodiments, the time to response is less than about 6 months after administration of the antibody-drug conjugate and the cervical cancer is a stage 4 cervical cancer.

In one embodiment of the methods or uses described herein, response to treatment with an antibody-drug conjugate or antigen-binding fragment thereof described herein is assessed by measuring the time of overall survival after administration of the antibody-drug conjugate. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the subject exhibits overall survival of at least about 6 months after administration of the antibody-drug conjugate. In some embodiments, the subject exhibits overall survival of at least about 10 months after administration of the antibody-drug conjugate. In some embodiments, the subject exhibits overall survival of at least about 11 months after administration of the antibody-drug conjugate. In some embodiments, the subject exhibits overall survival of at least about one year after administration of the antibody-drug conjugate. In some embodiments, the subject exhibits overall survival of at least about 13 months after administration of the antibody-drug conjugate. In some embodiments, the subject exhibits overall survival of at least about two years after administration of the antibody-drug conjugate. In some embodiments, the subject exhibits overall survival of at least about three years after administration of the antibody-drug conjugate. In some embodiments, the subject exhibits overall survival of at least about four years after administration of the antibody-drug conjugate. In some embodiments, the subject exhibits overall survival of at least about five years after administration of the antibody-drug conjugate. In some embodiments, the subject exhibits overall survival of at least about 10 months after administration of the antibody-drug conjugate. In some embodiments, the subject exhibits overall survival of at least about 10 months after administration of the antibody-drug conjugate and the patient received prior radiation to the pelvis. In some embodiments, the subject exhibits overall survival of at least about 10 months after administration of the antibody-drug conjugate and, the patient received prior radiation to the pelvis, and the patient experienced disease progression after the prior radiation to the pelvis. In some embodiments, the subject exhibits overall survival of at least about 10 months after administration of the antibody-drug conjugate and the patient has not received prior radiation to the pelvis. In some embodiments, the subject exhibits overall survival of at least about 10 months after administration of the antibody-drug conjugate and the patient received 1 prior systemic treatment regimen. In some embodiments, the subject exhibits overall survival of at least about 10 months after administration of the antibody-drug conjugate and the patient received 2 prior systemic treatment regimens. In some embodiments, the subject exhibits overall survival of at least about 10 months after administration of the antibody-drug conjugate, the patient received 1 prior systemic treatment regimen, and the patient experienced disease progression after the prior systemic treatment regimen. In some embodiments, the subject exhibits overall survival of at least about 10 months after administration of the antibody-drug conjugate, the patient received 2 prior systemic treatment regimens, and the patient experienced disease progression after the 2 prior systemic treatment regimens. In some embodiments, the prior systemic therapy was bevacizumab. In some embodiments, the prior systemic therapy was a chemotherapy. In some embodiments, the prior systemic therapy was a combination of a chemotherapy and bevacizumab. In some embodiments, the prior systemic therapy was a combination of doublet chemotherapy and bevacizumab. In some embodiments the doublet chemotherapy is a combination of paclitaxel and cisplatin. In some embodiments the doublet chemotherapy is a combination of paclitaxel and carboplatin. In some embodiments, the doublet chemotherapy is a combination of paclitaxel and topotecan. In some embodiments, the prior systemic therapy was a checkpoint inhibitor. In some embodiments, the prior systemic therapy was a pembrolizumab. In some embodiments, the subject exhibits overall survival of at least about 10 months after administration of the antibody-drug conjugate and the cervical cancer is a squamous cell carcinoma. In some embodiments, the subject exhibits overall survival of at least about 10 months after administration of the antibody-drug conjugate and the cervical cancer is an adenocarcinoma. In some embodiments, the subject exhibits overall survival of at least about 10 months after administration of the antibody-drug conjugate and the cervical cancer is an adenosquamous carcinoma. In some embodiments, the subject exhibits overall survival of at least about 10 months after administration of the antibody-drug conjugate and the subject has an ECOG score of 0. In some embodiments, the subject exhibits overall survival of at least about 10 months after administration of the antibody-drug conjugate and the subject has an ECOG score of 1. In some embodiments, the subject exhibits overall survival of at least about 10 months after administration of the antibody-drug conjugate and the subject has an ECOG score of 2. In some embodiments, the subject exhibits overall survival of at least about 10 months after administration of the antibody-drug conjugate and the subject has an ECOG score of 3. In some embodiments, the subject exhibits overall survival of at least about 10 months after administration of the antibody-drug conjugate and the subject has an ECOG score of 4. In some embodiments, the subject exhibits overall survival of at least about 10 months after administration of the antibody-drug conjugate and the cervical cancer cells are positive for membrane TF expression. In some embodiments, the subject exhibits overall survival of at least about 10 months after administration of the antibody-drug conjugate and the cervical cancer cells are positive for cytoplasmic TF expression. In some embodiments, positive TF expression is defined as ≥1% of cervical cancer cells expressing TF. In some embodiments, the subject exhibits overall survival of at least about 10 months after administration of the antibody-drug conjugate and the subject has a TF histology score (H-score) of at least 1. In some embodiments, the subject exhibits overall survival of at least about 10 months after administration of the antibody-drug conjugate and the subject is less than 65 years old. In some embodiments, the subject exhibits overall survival of at least about 10 months after administration of the antibody-drug conjugate and the subject is greater than or equal to 65 years old. In some embodiments, the subject exhibits overall survival of at least about 10 months after administration of the antibody-drug conjugate and the cervical cancer is a stage 3 cervical cancer. In some embodiments, the subject exhibits overall survival of at least about 10 months after administration of the antibody-drug conjugate and the cervical cancer is a stage 4 cervical cancer.

In one embodiment of the methods or uses described herein, response to treatment with an antibody-drug conjugate or antigen-binding fragment thereof described herein is assessed by measuring the duration of response to the antibody-drug conjugate after administration of the antibody-drug conjugate. In some embodiments, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the duration of response to the antibody-drug conjugate is at least about 6 months after administration of the antibody-drug conjugate. In some embodiments, the duration of response to the antibody-drug conjugate is at least about 7 months after administration of the antibody-drug conjugate. In some embodiments, the duration of response to the antibody-drug conjugate is at least about 8 months after administration of the antibody-drug conjugate. In some embodiments, the duration of response to the antibody-drug conjugate is at least about 10 months after administration of the antibody-drug conjugate. In some embodiments, the duration of response to the antibody-drug conjugate is at least about one year after administration of the antibody-drug conjugate. In some embodiments, the duration of response to the antibody-drug conjugate is at least about two years after administration of the antibody-drug conjugate. In some embodiments, the duration of response to the antibody-drug conjugate is at least about three years after administration of the antibody-drug conjugate. In some embodiments, the duration of response to the antibody-drug conjugate is at least about four years after administration of the antibody-drug conjugate. In some embodiments, the duration of response to the antibody-drug conjugate is at least about five years after administration of the antibody-drug conjugate. In some embodiments, the duration of response to the antibody-drug conjugate is at least about 8.3 months after administration of the antibody-drug conjugate. In some embodiments, the duration of response to the antibody-drug conjugate is at least about 6 months after administration of the antibody-drug conjugate and the patient received prior radiation to the pelvis. In some embodiments, the duration of response to the antibody-drug conjugate is at least about 6 months after administration of the antibody-drug conjugate, the patient received prior radiation to the pelvis, and the patient experienced disease progression after the prior radiation to the pelvis. In some embodiments, the duration of response to the antibody-drug conjugate is at least about 6 months after administration of the antibody-drug conjugate and the patient has not received prior radiation to the pelvis. In some embodiments, the duration of response to the antibody-drug conjugate is at least about 6 months after administration of the antibody-drug conjugate and the patient received 1 prior systemic treatment regimen. In some embodiments, the duration of response to the antibody-drug conjugate is at least about 6 months after administration of the antibody-drug conjugate and the patient received 2 prior systemic treatment regimens. In some embodiments, the duration of response to the antibody-drug conjugate is at least about 6 months after administration of the antibody-drug conjugate, the patient received 1 prior systemic treatment regimen, and the patient experienced disease progression after the prior systemic treatment regimen. In some embodiments, the duration of response to the antibody-drug conjugate is at least about 6 months after administration of the antibody-drug conjugate, the patient received 2 prior systemic treatment regimens, and the patient experienced disease progression after the 2 prior systemic treatment regimens. In some embodiments, the prior systemic therapy was bevacizumab. In some embodiments, the prior systemic therapy was a chemotherapy. In some embodiments, the prior systemic therapy was a combination of a chemotherapy and bevacizumab. In some embodiments, the prior systemic therapy was a combination of doublet chemotherapy and bevacizumab. In some embodiments the doublet chemotherapy is a combination of paclitaxel and cisplatin. In some embodiments the doublet chemotherapy is a combination of paclitaxel and carboplatin. In some embodiments, the doublet chemotherapy is a combination of paclitaxel and topotecan. In some embodiments, the prior systemic therapy was a checkpoint inhibitor. In some embodiments, the prior systemic therapy was a pembrolizumab. In some embodiments, the duration of response to the antibody-drug conjugate is at least about 6 months after administration of the antibody-drug conjugate and the cervical cancer is a squamous cell carcinoma. In some embodiments, the duration of response to the antibody-drug conjugate is at least about 6 months after administration of the antibody-drug conjugate and the cervical cancer is an adenocarcinoma. In some embodiments, the duration of response to the antibody-drug conjugate is at least about 6 months after administration of the antibody-drug conjugate and the cervical cancer is an adenosquamous carcinoma. In some embodiments, the duration of response to the antibody-drug conjugate is at least about 6 months after administration of the antibody-drug conjugate and the subject has an ECOG score of 0. In some embodiments, the duration of response to the antibody-drug conjugate is at least about 6 months after administration of the antibody-drug conjugate and the subject has an ECOG score of 1. In some embodiments, the duration of response to the antibody-drug conjugate is at least about 6 months after administration of the antibody-drug conjugate and the subject has an ECOG score of 2. In some embodiments, the duration of response to the antibody-drug conjugate is at least about 6 months after administration of the antibody-drug conjugate and the subject has an ECOG score of 3. In some embodiments, the duration of response to the antibody-drug conjugate is at least about 6 months after administration of the antibody-drug conjugate and the subject has an ECOG score of 4. In some embodiments, the duration of response to the antibody-drug conjugate is at least about 6 months after administration of the antibody-drug conjugate and the cervical cancer cells are positive for membrane TF expression. In some embodiments, the duration of response to the antibody-drug conjugate is at least about 6 months after administration of the antibody-drug conjugate and the cervical cancer cells are positive for cytoplasmic TF expression. In some embodiments, positive TF expression is defined as ≥1% of cervical cancer cells expressing TF. In some embodiments, the duration of response to the antibody-drug conjugate is at least about 6 months after administration of the antibody-drug conjugate and the subject has a TF histology score (H-score) of at least 1. In some embodiments, the duration of response to the antibody-drug conjugate is at least about 6 months after administration of the antibody-drug conjugate and the subject is less than 65 years old. In some embodiments, the duration of response to the antibody-drug conjugate is at least about 6 months after administration of the antibody-drug conjugate and the subject is greater than or equal to 65 years old. In some embodiments, the duration of response to the antibody-drug conjugate is at least about 6 months after administration of the antibody-drug conjugate and the cervical cancer is a stage 3 cervical cancer. In some embodiments, the duration of response to the antibody-drug conjugate is at least about 6 months after administration of the antibody-drug conjugate and the cervical cancer is a stage 4 cervical cancer.

In some embodiments, a method of treating cervical cancer with an antibody-drug conjugates or antigen-binding fragments thereof described herein results in an improvement in one or more therapeutic effects in the subject after administration of the antibody-drug conjugate relative to a baseline. In some embodiments, the one or more therapeutic effects is the size of the tumor derived from the cervical cancer, the objective response rate, the duration of response, the time to response, progression free survival, overall survival, or any combination thereof. In one embodiment, the one or more therapeutic effects is the size of the tumor derived from the cervical cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, and the size of a tumor derived from the cervical cancer in the subject is reduced by at least about 30%. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, and a tumor derived from the cervical cancer in the subject regresses by at least about 30%. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, and the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, and the time to response is less than about is less than about 6 months after administration of the antibody-drug conjugate, optionally, wherein the time to response is less than about 4 months, about 2 months, 1.4 months, or about 1.2 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, and the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, and the duration of response to the antibody-drug conjugate is at least about 6 months after administration of the antibody-drug conjugate, optionally, wherein the duration of response to the antibody-drug conjugate is at least about 7 months, 8 months or about 10 months after administration of the antibody-drug conjugate. In some embodiments, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate, and the time to response is less than about is less than about 6 months after administration of the antibody-drug conjugate, optionally, wherein the time to response is less than about 4 months, about 2 months, 1.4 months, or about 1.2 months after administration of the antibody-drug conjugate. In some embodiments, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate, and the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate, and the duration of response to the antibody-drug conjugate is at least about 6 months after administration of the antibody-drug conjugate, optionally, wherein the duration of response to the antibody-drug conjugate is at least about 7 months, 8 months or about 10 months after administration of the antibody-drug conjugate. In some embodiments, the time to response is less than about 6 months after administration of the antibody-drug conjugate, optionally, wherein the time to response is less than about 4 months, about 2 months, 1.4 months, or about 1.2 months after administration of the antibody-drug conjugate, and the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the time to response is less than about is less than about 6 months after administration of the antibody-drug conjugate, optionally, wherein the time to response is less than about 4 months, about 2 months, 1.4 months, or about 1.2 months after administration of the antibody-drug conjugate, and the duration of response to the antibody-drug conjugate is at least about 6 months after administration of the antibody-drug conjugate, optionally, wherein the duration of response to the antibody-drug conjugate is at least about 7 months, 8 months or about 10 months after administration of the antibody-drug conjugate. In some embodiments, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate, and the duration of response to the antibody-drug conjugate is at least about 6 months after administration of the antibody-drug conjugate, optionally, wherein the duration of response to the antibody-drug conjugate is at least about 7 months, 8 months or about 10 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate, and the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate, and the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate, and the time to response is less than about is less than about 6 months after administration of the antibody-drug conjugate, optionally, wherein the time to response is less than about 4 months, about 2 months, 1.4 months, or about 1.2 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate, and the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate, and the time to response is less than about is less than about 6 months after administration of the antibody-drug conjugate, optionally, wherein the time to response is less than about 4 months, about 2 months, 1.4 months, or about 1.2 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate, and the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate, and the time to response is less than about is less than about 6 months after administration of the antibody-drug conjugate, optionally, wherein the time to response is less than about 4 months, about 2 months, 1.4 months, or about 1.2 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate, and the time to response is less than about is less than about 6 months after administration of the antibody-drug conjugate, optionally, wherein the time to response is less than about 4 months, about 2 months, 1.4 months, or about 1.2 months after administration of the antibody-drug conjugate. In some embodiments, the objective response rate is between about 13% and about 35%, optionally, wherein the objective response rate is at least about 14%, about 19%, about 21%, 23.8%, about 24%, about 25%, about 26%, about 28%, about 30%, or about 33%, the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate, the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate, the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate, and the time to response is less than about 6 months after administration of the antibody-drug conjugate, optionally, wherein the time to response is less than about 4 months, about 2 months, 1.4 months, or about 1.2 months after administration of the antibody-drug conjugate. In some embodiments, the subject has an ECOG score of 0. In some embodiments, the subject has an ECOG score of 1. In some embodiments, the subject is less than 65 years old. In some embodiments, the subject has been previously treated with bevacizumab. In some embodiments, the subject has not been previously treated with bevacizumab. In some embodiments, the subject has been previously treated with paclitaxel and cisplatin. In some embodiments, the subject has been previously treated with paclitaxel and carboplatin. In some embodiments, the subject has been previously treated with paclitaxel and topotecan. In some embodiments, the subject has been previously treated with bevacizumab paclitaxel, and cisplatin. In some embodiments, the subject has been previously treated with bevacizumab, paclitaxel, and carboplatin. In some embodiments, the subject has been previously treated with bevacizumab, paclitaxel, and topotecan.

E. Adverse Events

In one aspect, a method of treating cervical cancer with an antibody-drug conjugates or antigen-binding fragments thereof described herein results in the subject developing one or more adverse events. In some embodiments, the subject is administered an additional therapeutic agent to eliminate or reduce the severity of the adverse event. In some embodiments, the one or more adverse events the subject develops is anemia, abdominal pain, a bleeding-related adverse event, hypokalemia, hyponatremia, epistaxis, fatigue, nausea, alopecia, conjunctivitis, constipation, decreased appetite, diarrhea, vomiting, peripheral neuropathy, general physical health deterioration, or any combination thereof. In some embodiments, the adverse event the subject develops is anemia. In some embodiments, the adverse event the subject develops is abdominal pain. In some embodiments, the adverse event the subject develops is a bleeding-related adverse event. In some embodiments, the adverse event the subject develops is hypokalemia. In some embodiments, the adverse event the subject develops is hyponatremia. In some embodiments, the adverse event the subject develops is epistaxis. In some embodiments, the adverse event the subject develops is fatigue. In some embodiments, the adverse event the subject develops is nausea. In some embodiments, the adverse event the subject develops is alopecia. In some embodiments, the adverse event the subject develops is conjunctivitis. In some embodiments, the adverse event the subject develops is constipation. In some embodiments, the adverse event the subject develops is decreased appetite. In some embodiments, the adverse event the subject develops is diarrhea. In some embodiments, the adverse event the subject develops is vomiting. In some embodiments, the adverse event the subject develops is peripheral neuropathy. In some embodiments, the adverse event the subject develops is general physical health deterioration. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a treatment-emergent adverse event related to treatment. In some embodiments, the one or more adverse events is a serious adverse event. In some embodiments, the one or more adverse events is conjunctivitis and/or keratitis and the additional therapeutic agent is a preservative-free lubricating eye drop, an ocular vasoconstrictor, a steroid eye drop, or any combination thereof. In some embodiments, the one or more adverse events is conjunctivitis and keratitis and the additional therapeutic agent is a preservative-free lubricating eye drop, an ocular vasoconstrictor, a steroid eye drop, or any combination thereof. In some embodiments, the one or more adverse events is conjunctivitis and the additional therapeutic agent is a preservative-free lubricating eye drop, an ocular vasoconstrictor, a steroid eye drop, or any combination thereof. In some embodiments, the one or more adverse events is keratitis and the additional therapeutic agent is a preservative-free lubricating eye drop, an ocular vasoconstrictor, a steroid eye drop, or any combination thereof. In some of any of the embodiments herein, the subject is administered a treatment with the additional therapeutic agent to eliminate or reduce the severity of the adverse event (e.g., conjunctivitis and/or keratitis). In some embodiments, the treatment is an ocular vasoconstrictor. In some embodiments, the ocular vasoconstrictor is brimonidine tartrate 0.2% eye drops. In some embodiments, 3 drops of brimonidine tartrate 0.2% eye drops are administered immediately prior to the start of administration of the antibody-drug conjugate. In some embodiments, the treatment is eye cooling pads (e.g. THERA PEARL Eye Mask or similar). In some embodiments, the eye cooling pads are applied during administration of the antibody-drug conjugate. In some embodiments, the eye cooling pads are applied 5 minutes before the start of administration of the antibody-drug conjugate and remain on during the entire infusion of the antibody-drug conjugate. In some embodiments, the eye cooling pads are applied 5 minutes before the start of administration of the antibody-drug conjugate and remain on during the entire infusion of the antibody-drug conjugate and for at least about 30 minutes after the end of the infusion of the antibody-drug conjugate. In some embodiments, the treatment is steroid eye drops. In some embodiments, the steroid eye drops are dexamethasone 0.1% eye drops. In some embodiments, the steroid eye drops are applied before and after each infusion of the antibody-drug conjugate for a total of 4 days. In some embodiments, the steroid eye drops are applied starting about 24 hours prior to the start of infusion of the antibody-drug conjugate and are applied until about 72 hours after the end of infusion of the antibody-drug conjugate. In some embodiments, the steroid eye drops are administered as 1 drop in each eye 3 times per day. In some embodiments, the treatment is lubricating eye drops. In some embodiments, the lubricating eye drops are administered from the first infusion of the antibody-drug conjugate and continue to be administered until 30 days after the last infusion of the antibody-drug conjugate. In some embodiments, the lubricating eye drops are administered daily. In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

In one aspect, the subject treated with an antibody-drug conjugates or antigen-binding fragments thereof described herein is at risk of developing one or more adverse events. In some embodiments, the subject is administered an additional therapeutic agent to prevent the development of the adverse event or to reduce the severity of the adverse event. In some embodiments, the one or more adverse events the subject is at risk of developing is anemia, a bleeding-related adverse event, abdominal pain, hypokalemia, hyponatremia, epistaxis, fatigue, nausea, alopecia, conjunctivitis, constipation, decreased appetite, diarrhea, vomiting, peripheral neuropathy, general physical health deterioration, or any combination thereof. In some embodiments, the adverse event the subject is at risk of developing is anemia. In some embodiments, the adverse event the subject is at risk of developing is abdominal pain. In some embodiments, the adverse event the subject is at risk of developing is a bleeding-related adverse event. In some embodiments, the adverse event the subject is at risk of developing is hypokalemia. In some embodiments, the adverse event the subject is at risk of developing is hyponatremia. In some embodiments, the adverse event the subject is at risk of developing is epistaxis. In some embodiments, the adverse event the subject is at risk of developing is fatigue. In some embodiments, the adverse event the subject is at risk of developing is nausea. In some embodiments, the adverse event the subject is at risk of developing is alopecia. In some embodiments, the adverse event the subject is at risk of developing is conjunctivitis. In some embodiments, the adverse event the subject is at risk of developing is constipation. In some embodiments, the adverse event the subject is at risk of developing is decreased appetite. In some embodiments, the adverse event the subject is at risk of developing is diarrhea. In some embodiments, the adverse event the subject is at risk of developing is vomiting. In some embodiments, the adverse event the subject is at risk of developing is peripheral neuropathy. In some embodiments, the adverse event the subject is at risk of developing is general physical health deterioration. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a treatment-emergent adverse event related to treatment. In some embodiments, the one or more adverse events is a serious adverse event. In some embodiments, the one or more adverse events is conjunctivitis and/or keratitis and the additional agent is a preservative-free lubricating eye drop, an ocular vasoconstrictor, a steroid eye drop, or any combination thereof. In some embodiments, the one or more adverse events is conjunctivitis and keratitis and the additional agent is a preservative-free lubricating eye drop, an ocular vasoconstrictor, a steroid eye drop, or any combination thereof. In some embodiments, the one or more adverse events is conjunctivitis and the additional agent is a preservative-free lubricating eye drop, an ocular vasoconstrictor, a steroid eye drop, or any combination thereof. In some embodiments, the one or more adverse events is keratitis and the additional agent is a preservative-free lubricating eye drop, an ocular vasoconstrictor, a steroid eye drop, or any combination thereof. In some of any of the embodiments herein, the subject is administered a treatment with the additional therapeutic agent to prevent the development of the adverse event or to reduce the severity of the adverse event (e.g., conjunctivitis and/or keratitis). In some embodiments, the treatment is an ocular vasoconstrictor. In some embodiments, the ocular vasoconstrictor is brimonidine tartrate 0.2% eye drops. In some embodiments, 3 drops of brimonidine tartrate 0.2% eye drops are administered immediately prior to the start of administration of the antibody-drug conjugate. In some embodiments, the treatment is eye cooling pads (e.g. THERA PEARL Eye Mask or similar). In some embodiments, the eye cooling pads are applied during administration of the antibody-drug conjugate. In some embodiments, the eye cooling pads are applied 5 minutes before the start of administration of the antibody-drug conjugate and remain on during the entire infusion of the antibody-drug conjugate. In some embodiments, the eye cooling pads are applied 5 minutes before the start of administration of the antibody-drug conjugate and remain on during the entire infusion of the antibody-drug conjugate and for at least about 30 minutes after the end of the infusion of the antibody-drug conjugate. In some embodiments, the treatment is steroid eye drops. In some embodiments, the steroid eye drops are dexamethasone 0.1% eye drops. In some embodiments, the steroid eye drops are applied before and after each infusion of the antibody-drug conjugate for a total of 4 days. In some embodiments, the steroid eye drops are applied starting about 24 hours prior to the start of infusion of the antibody-drug conjugate and are applied until about 72 hours after the end of infusion of the antibody-drug conjugate. In some embodiments, the steroid eye drops are administered as 1 drop in each eye 3 times per day. In some embodiments, the treatment is lubricating eye drops. In some embodiments, the lubricating eye drops are administered from the first infusion of the antibody-drug conjugate and continue to be administered until 30 days after the last infusion of the antibody-drug conjugate. In some embodiments, the lubricating eye drops are administered daily. In some embodiments, the one or more adverse events is a recurrent infusion related reaction and the additional agent is an antihistamine, acetaminophen and/or a corticosteroid. In some embodiments, the one or more adverse events is neutropenia and the additional agent is growth factor support (G-CSF).

### IV. Compositions

In some aspects, also provided herein are compositions (e.g., pharmaceutical composition) comprising any of the anti-TF antibody-drug conjugates described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1 to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

Formulations comprising an anti-TF antibody-conjugate described herein for use in methods of treatment provided herein are described in WO2015/075201. In some embodiments, an anti-TF antibody-drug conjugate described herein is in a formulation comprising the anti-TF antibody drug conjugate, histidine, sucrose, and D-mannitol, wherein the formulation has a pH of about 6.0. In some embodiments, an anti-TF antibody-drug conjugate described herein is in a formulation comprising the anti-TF antibody drug conjugate at a concentration of about 10 mg/ml, histidine at a concentration of about 30 mM, sucrose at a concentration of about 88 mM, D-mannitol at a concentration of about 165 mM, wherein the formulation has a pH of about 6.0. In some embodiments, an anti-TF antibody-drug conjugate described herein is in a formulation comprising the anti-TF antibody drug conjugate at a concentration of 10 mg/ml, histidine at a concentration of 30 mM, sucrose at a concentration of 88 mM, D-mannitol at a concentration of 165 mM, wherein the formulation has a pH of 6.0. In some embodiments, the formulation comprises tisotumab vedotin at a concentration of 10 mg/ml, histidine at a concentration of 30 mM, sucrose at a concentration of 88 mM, D-mannitol at a concentration of 165 mM, wherein the formulation has a pH of 6.0.

In some embodiments provided herein, a formulation comprising the anti-TF antibody-conjugate described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-TF antibody-drug conjugates or antigen-binding fragments thereof as described herein for use in a method of treating cervical cancer as described herein. In some aspects, provided herein are compositions comprising a population of antibody-drug conjugates, wherein the antibody-drug conjugates comprise a linker attached to MMAE, wherein the antibody-drug conjugate has the following structure:

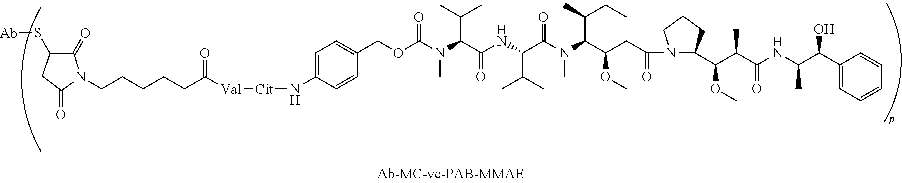

wherein p denotes a number from 1 to 8, S represents a sulphydryl residue of the anti-TF antibody or antigen-binding fragment thereof, and Ab designates the anti-TF antibody or antigen-binding fragment thereof as described herein, such as tisotumab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for p.

In some embodiments, a composition comprising an antibody-drug conjugate as described herein is coadministered with one or additional therapeutic agents. In some embodiments the coadministration is simultaneous or sequential. In some embodiments, the antibody-drug conjugate as described herein is administered simultaneously with the one or more additional therapeutic agents. In some embodiments, simultaneous means that the antibody-drug conjugate and the one or more therapeutic agents are administered to the subject less than one hour apart, such as less than about 30 minutes apart, less than about 15 minutes apart, less than about 10 minutes apart or less than about 5 minutes apart. In some embodiments, the antibody-drug conjugate as described herein is administered sequentially with the one or more additional therapeutic agents. In some embodiments, sequential administration means that the antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, a composition comprising an antibody-drug conjugate as described herein is coadministered with one or more therapeutic agents to eliminate or reduce the severity of one or more adverse events. In some embodiments, a composition comprising an antibody-drug conjugate as described herein is coadministered with one or more therapeutic agents to prevent the development of the adverse event or to reduce the severity of the adverse event.

In some embodiments, a composition comprising an antibody-drug conjugate as described herein is coadministered with one or more therapeutic agents to eliminate or reduce the severity of one or more adverse events. In some embodiments the coadministration is simultaneous or sequential. In some embodiments, the antibody-drug conjugate as described herein is administered simultaneously with the one or more therapeutic agents to eliminate or reduce the severity of one or more adverse events. In some embodiments, simultaneous means that the antibody-drug conjugate and the one or more therapeutic agents to eliminate or reduce the severity of one or more adverse events are administered to the subject less than one hour apart, such as less than about 30 minutes apart, less than about 15 minutes apart, less than about 10 minutes apart or less than about 5 minutes apart. In some embodiments, the antibody-drug conjugate as described herein is administered sequentially with the one or more therapeutic agents to eliminate or reduce the severity of one or more adverse events. In some embodiments, sequential administration means that the antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the antibody-drug conjugate is administered prior to the one or more therapeutic agents to eliminate or reduce the severity of one or more adverse events. In some embodiments, the one or more therapeutic agents to eliminate or reduce the severity of one or more adverse events is administered prior to the antibody-drug conjugate.

### V. Articles of Manufacture and Kits

In another aspect, an article of manufacture or kit is provided which comprises an anti-TF antibody-drug conjugate described herein. The article of manufacture or kit may further comprise instructions for use of the antibody in the methods of the invention. Thus, in certain embodiments, the article of manufacture or kit comprises instructions for the use of an anti-TF antibody-drug conjugate in methods for treating cervical cancer in a subject comprising administering to the subject an effective amount of an anti-TF antibody-drug conjugate. In some embodiments, the cervical cancer is advanced cervical cancer, such as grade 3 cervical cancer or grade 4 cervical cancer. In some embodiments, the advanced cervical cancer is metastatic cancer. In some embodiments, the cervical cancer is metastatic cancer and recurrent cancer. In some embodiments the cervical cancer is recurrent cancer. In some embodiments, the subject has been previously treated with one or more therapeutic agents and did not respond to the treatment, relapsed after treatment, or experienced disease progression during treatment. In some embodiments herein of the previous treatment, the one or more therapeutic agents is not the antibody-drug conjugate. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cervical cancer in a subject such as cervical cancer described herein (e.g., advanced cervical cancer such as grade 3 or grade 4 or metastatic cervical cancer). The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-TF antibody-drug conjugate is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein.

The article of manufacture or kit herein optionally further comprises a container comprising a second medicament, wherein the second medicament is for eliminating or reducing the severity of one or more adverse events, wherein the anti-TF antibody-drug conjugate is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein, for example wherein the label or package insert indicates that the anti-TF antibody-drug conjugate is to be administered first, followed by administration of the second medicament.

In some embodiments, the anti-TF antibody-drug conjugate is present in the container as a lyophilized powder. In some embodiments, the lyophilized powder is in a hermetically sealed container, such as a vial, an ampoule or sachet, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

### VI. Exemplary Embodiments

### Methods of Treatment

1A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has been previously treated with bevacizumab.
2A. The method of embodiment 1A, wherein the subject has an ECOG score of 0.
3A. The method of embodiment 1A, wherein the subject has an ECOG score of 1.
4A. The method of any one of embodiments 1A-3A, wherein the subject is less than 65 years old.
5A. The method of any one of embodiments 1A-4A, wherein the subject has been previously treated with:
a) paclitaxel and cisplatin,
b) paclitaxel and carboplatin, or
c) paclitaxel and topotecan.
   6A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has not been previously treated with bevacizumab.
   7A. The method of embodiment 6A, wherein the subject has an ECOG score of 0.
   8A. The method of embodiment 6A, wherein the subject has an ECOG score of 1.
   9A. The method of any one of embodiments 6A-8A, wherein the subject is less than 65 years old.
   10A. The method of any one of embodiments 6A-9A, wherein the subject has been previously treated with:
a) paclitaxel and cisplatin,
b) paclitaxel and carboplatin, or
c) paclitaxel and topotecan.
   11A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has an Eastern Cooperative Oncology Group (ECOG) score of 0.
   12A. The method of embodiment 11A, wherein the subject is less than 65 years old.
   13A. The method of any embodiment 11A or 12A, wherein the subject has been previously treated with:
a) paclitaxel and cisplatin,
b) paclitaxel and carboplatin, or
c) paclitaxel and topotecan.
   14A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has an ECOG score of 1.
   15A. The method of embodiment 14A, wherein the subject is less than 65 years old.
   16A. The method of any embodiment 14A or 15A, wherein the subject has been previously treated with:
a) paclitaxel and cisplatin,
b) paclitaxel and carboplatin, or
c) paclitaxel and topotecan.
   17A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject is less than 65 years old.
   18A. The method of embodiment 17A, wherein the subject has an ECOG score of 0.
   19A. The method of embodiment 17A, wherein the subject has an ECOG score of 1.
   20A. The method of any one of embodiments 17A-19A, wherein the subject has been previously treated with:
a) paclitaxel and cisplatin,
b) paclitaxel and carboplatin, or
c) paclitaxel and topotecan.
   21A. The method of any one of embodiments 1A-20A, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   22A. The method of any one of embodiments 1A-21A, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   23A. The method of any one of embodiments 1A-22A, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   24A. The method of any one of embodiments 1A-23A, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   25A. The method of any one of embodiments 1A-24A, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   26A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   27A. The method of embodiment 26A, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   28A. The method of embodiment 26A or 27A, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   29A. The method of any one of embodiments 26A-28A, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   30A. The method of any one of embodiments 26A-29A, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   31A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   32A. The method of embodiment 31A, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   33A. The method of embodiment 31A or 32A, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   34A. The method of any one of embodiments 31A-33A, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   35A. The method of any one of embodiments 31A-34A, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   36A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   37A. The method of embodiment 36A, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   38A. The method of embodiment 36A or 37A, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   39A. The method of any one of embodiments 36A-38A, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   40A. The method of any one of embodiments 36A-39A, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   41A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   42A. The method of embodiment 41A, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   43A. The method of embodiment 41A or 42A, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   44A. The method of any one of embodiments 41A-43A, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   45A. The method of any one of embodiments 41A-44A, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   46A. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   47A. The method of embodiment 46A, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   48A. The method of embodiment 46A or 47A, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   49A. The method of any one of embodiments 46A-48A, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   50A. The method of any one of embodiments 46A-49A, wherein the objective response rate is at least about between about 13% and about 35%, optionally, wherein the objective response rate is at least about 14%, about 19%, about 21%, 23.8%, about 24%, about 25%, about 26%, about 28%, about 30%, or about 33%.
   51A. The method of any one of embodiments 26A-50A, wherein the subject has been previously treated with bevacizumab.
   52A. The method of any one of embodiments 26A-50A, wherein the subject has not been previously treated with bevacizumab.
   53A. The method of any one of embodiments 26A-52A, wherein the subject has experienced disease progression during or after treatment with:
a) paclitaxel and cisplatin,
b) paclitaxel and carboplatin,
c) paclitaxel and topotecan,
d) bevacizumab, paclitaxel, and cisplatin,
e) bevacizumab, paclitaxel, and carboplatin, or
f) bevacizumab, paclitaxel, and topotecan.
   54A. The method of any one of embodiments 26A-53A, wherein the subject is less than 65 years old.
   55A. The method of any one of embodiments 26A-54A, wherein the subject has an ECOG score of 0.
   56A. The method of any one of embodiments 26A-54A, wherein the subject has an ECOG score of 1.
   57A. The method of any one of embodiments 1A-56A, wherein the cervical cancer is an adenocarcinoma.
   58A. The method of any one of embodiments 1A-56A, wherein the cervical cancer is an adenosquamous carcinoma.
   59A. The method of any one of embodiments 1A-56A, wherein the cervical cancer is a squamous cell carcinoma.
   60A. The method of any one of embodiments 1A-56A, wherein the cervical cancer is a non-squamous cell carcinoma.
   61A. The method of any one of embodiments 1A-60A, wherein the dose is about 2.0 mg/kg.
   62A. The method of any one of embodiments 1A-60A, wherein the dose is 2.0 mg/kg.
   63A. The method of any one of embodiments 1A-62A, wherein the antibody-drug conjugate is administered once about every 1 week, 2 weeks, 3 weeks or 4 weeks.
   64A. The method of any one of embodiments 1A-62A, wherein the antibody-drug conjugate is administered once about every 3 weeks.
   65A. The method of any one of embodiments 1A-64A, wherein the antibody-drug conjugate is administered once every 3 weeks.
   66A. The method of any one of embodiments 1A-65A, wherein the cervical cancer is recurrent or metastatic cervical cancer.
   67A. The method of any one of embodiments 1A-66A, wherein the subject has been previously treated with one or more therapeutic agents and did not respond to the treatment, wherein the one or more therapeutic agents is not the antibody-drug conjugate.
   68A. The method of any one of embodiments 1A-66A, wherein the subject has been previously treated with one or more therapeutic agents and relapsed after the treatment, wherein the one or more therapeutic agents is not the antibody-drug conjugate.
   69A. The method of any one of embodiments 1A-66A, wherein the subject has been previously treated with one or more therapeutic agents and has experienced disease progression during treatment, wherein the one or more therapeutic agents is not the antibody-drug conjugate.
   70A. The method of any one of embodiments 67A-69A, wherein the one or more therapeutic agents is a platinum-based therapeutic agent.
   71A. The method of any one of embodiments 67A-69A, wherein the one or more therapeutic agents is selected from the group consisting of: paclitaxel, cisplatin, carboplatin, topotecan, gemcitabine, fluorouracil, ixabepilone, imatinib mesylate, docetaxel, gefitinib, nab-paclitaxel, pemetrexed, vinorelbine, doxil, cetuximab, pembrolizumab, nivolumab and bevacizumab.
   72A. The method of any one of embodiments 1A-71A, wherein the subject is not a candidate for curative therapy.
   73A. The method of embodiment 72A, wherein the curative therapy comprises radiotherapy and/or exenterative surgery.
   74A. The method of any one of embodiments 1A-71A, wherein the subject has received prior radiation to the pelvis.
   75A. The method of any one of embodiments 1A-71A, wherein the subject has not received prior radiation to the pelvis.
   76A. The method of any one of embodiments 1A-75A, wherein the subject has received one prior line of systemic therapy for relapsed, recurrent or metastatic cancer.
   77A. The method of any one of embodiments 1A-75A, wherein the subject has received two prior lines of systemic therapy for relapsed, recurrent or metastatic cancer.
   78A. The method of any one of embodiments 76A or 77A, wherein the subject did not respond to treatment with the prior systemic therapy.
   79A. The method of any one of embodiments 76A or 77A, wherein the subject relapsed after treatment with the prior systemic therapy.
   80A. The method of any one of embodiments 1A-79A, wherein the cervical cancer is an advanced stage cervical cancer, such as a stage 3 or stage 4 cervical cancer, such as metastatic cervical cancer.
   81A. The method of any one of embodiments 1A-80A, wherein the cervical cancer is recurrent cervical cancer.
   82A. The method of any one of embodiments 1A-81A, wherein the monomethyl auristatin is monomethyl auristatin E (MMAE).
   83A. The method of any one of embodiments 1A-82A, wherein the anti-TF antibody or antigen-binding fragment thereof of the antibody-drug conjugate is a monoclonal antibody or a monoclonal antigen-binding fragment thereof.
   84A. The method of any one of embodiments 1A-83A, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
   wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6, wherein the CDRs of the anti-TF antibody or antigen-binding fragment thereof of the antibody-drug conjugate are defined by the IMGT numbering scheme.
   85A. The method of any one of embodiments 1A-84A, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
   86A. The method of any one of embodiments 1A-85A, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
   87A. The method of any one of embodiments 1A-86A, wherein the anti-TF antibody of the antibody-drug conjugate is tisotumab.
   88A. The method of any one of embodiments 1A-87A, wherein the antibody-drug conjugate further comprises a linker between the anti-TF antibody or antigen-binding fragment thereof and the monomethyl auristatin.
   89A. The method of embodiment 88A, wherein the linker is a cleavable peptide linker.
   90A. The method of embodiment 89A, wherein the cleavable peptide linker has a formula: -MC-vc-PAB-, wherein:
a) MC is:

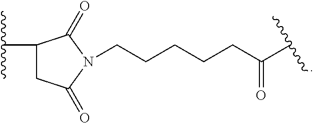 [SMILES: *C(=O)CCCCCN1C(=O)CC(*)C1=O]

b) vc is the dipeptide valine-citrulline, and
c) PAB is:

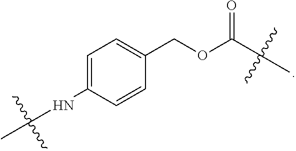 [SMILES: *Nc1ccc(COC(*)=O)cc1]

91A. The method of any one of embodiments 88A-90A, wherein the linker is attached to sulphydryl residues of the anti-TF antibody obtained by partial reduction or full reduction of the anti-TF antibody or antigen-binding fragment thereof.
92A. The method of embodiment 91A, wherein the linker is attached to MMAE, wherein the antibody-drug conjugate has the following-structure:

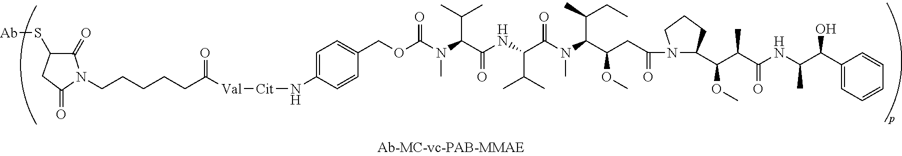

wherein p denotes a number from 1 to 8, S represents a sulphydryl residue of the anti-TF antibody, and Ab designates the anti-TF antibody or antigen-binding fragment thereof.
93A. The method of embodiment 92A, wherein the average value of p in a population of the antibody-drug conjugates is about 4.
94A. The method of any one of embodiments 1A-93A, wherein the antibody-drug conjugate is tisotumab vedotin or a biosimilar thereof.
95A. The method of any one of embodiments 1A-94A, wherein the route of administration for the antibody-drug conjugate is intravenous.
96A. The method of any one of embodiments 1A-95A, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF.
97A. The method of any one of embodiments 1A-96A, wherein the subject has a TF histology score (H-score) of at least 1.
98A. The method of any one of embodiments 1A-97A, wherein the subject has one or more adverse events and is further administered an additional therapeutic agent to eliminate or reduce the severity of the one or more adverse events.
99A. The method of any one of embodiments 1A-97A, wherein the subject is at risk of developing one or more adverse events and is further administered an additional therapeutic agent to prevent or reduce the severity of the one or more adverse events.
100A. The method of any one of embodiments 1A-98A, wherein the subject has one or more adverse events and the dose of the antibody drug conjugate is reduced following the one or more adverse events.
101A. The method of embodiment 100A, wherein the dose is reduced from 2.0 mg/kg to 1.3 mg/kg.
102A. The method of embodiment 100A or 102A, wherein the dose is reduced from 1.3 mg/kg to 0.9 mg/kg.
103A. The method of any one of embodiments 98A-102A, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.
104A. The method of any one of embodiments 98A-103A, wherein the one or more adverse events is a grade 3 or greater adverse event.
105A. The method of any one of embodiments 98A-103A, wherein the one or more adverse events is a serious adverse event.
106A. The method of embodiment 98A or embodiment 98A, wherein the one or more adverse events is conjunctivitis and/or keratitis and the additional agent is a preservative-free lubricating eye drop, an ocular vasoconstrictor and/or a steroid eye drop.
107A. The method of any one of embodiments 1A-106A, wherein the antibody-drug conjugate is administered as a monotherapy.
108A. The method of any one of embodiments 1A-107A, wherein the subject is a human.
109A. The method of any one of embodiments 1A-108A, wherein the antibody-drug conjugate is in a pharmaceutical composition comprising the antibody-drug conjugate and a pharmaceutical acceptable carrier.

### Antibody-Drug Conjugate for Use

1B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has been previously treated with bevacizumab.
2B. The antibody-drug conjugate for use of embodiment 1B, wherein the subject has an ECOG score of 0.
3B. The antibody-drug conjugate for use of embodiment 1B, wherein the subject has an ECOG score of 1.
4B. The antibody-drug conjugate for use of any one of embodiments 1B-3B, wherein the subject is less than 65 years old.
5B. The antibody-drug conjugate for use of any one of embodiments 1B-4B, wherein the subject has been previously treated with:
a) paclitaxel and cisplatin,
b) paclitaxel and carboplatin, or
c) paclitaxel and topotecan.
   6B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has not been previously treated with bevacizumab.
   7B. The antibody-drug conjugate for use of embodiment 6B, wherein the subject has an ECOG score of 0.
   8B. The antibody-drug conjugate for use of embodiment 6B, wherein the subject has an ECOG score of 1.
   9B. The antibody-drug conjugate for use of any one of embodiments 6B-8B, wherein the subject is less than 65 years old.
   10B. The antibody-drug conjugate for use of any one of embodiments 6B-9B, wherein the subject has been previously treated with:
a) paclitaxel and cisplatin,
b) paclitaxel and carboplatin, or
c) paclitaxel and topotecan.
   11B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has an Eastern Cooperative Oncology Group (ECOG) score of 0.
   12B. The antibody-drug conjugate for use of embodiment 11B, wherein the subject is less than 65 years old.
   13B. The antibody-drug conjugate for use of any embodiment 11B or 12B, wherein the subject has been previously treated with:
a) paclitaxel and cisplatin,
b) paclitaxel and carboplatin, or
c) paclitaxel and topotecan.
   14B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has an ECOG score of 1.
   15B. The antibody-drug conjugate for use of embodiment 14B, wherein the subject is less than 65 years old.
   16B. The antibody-drug conjugate for use of any embodiment 14B or 15B, wherein the subject has been previously treated with:
a) paclitaxel and cisplatin,
b) paclitaxel and carboplatin, or
c) paclitaxel and topotecan.
   17B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject is less than 65 years old.
   18B. The antibody-drug conjugate for use of embodiment 17B, wherein the subject has an ECOG score of 0.
   19B. The antibody-drug conjugate for use of embodiment 17B, wherein the subject has an ECOG score of 1.
   20B. The antibody-drug conjugate for use of any one of embodiments 17B-19B, wherein the subject has been previously treated with:
a) paclitaxel and cisplatin,
b) paclitaxel and carboplatin, or
c) paclitaxel and topotecan.
   21B. The antibody-drug conjugate for use of any one of embodiments 1B-20B, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   22B. The antibody-drug conjugate for use of any one of embodiments 1B-21B, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   23B. The antibody-drug conjugate for use of any one of embodiments 1B-22B, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   24B. The antibody-drug conjugate for use of any one of embodiments 1B-23B, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   25B. The antibody-drug conjugate for use of any one of embodiments 1B-24B, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   26B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   27B. The antibody-drug conjugate for use of embodiment 26B, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   28B. The antibody-drug conjugate for use of embodiment 26B or 27B, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   29B. The antibody-drug conjugate for use of any one of embodiments 26B-28B, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   30B. The antibody-drug conjugate for use of any one of embodiments 26B-29B, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   31B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   32B. The antibody-drug conjugate for use of embodiment 31B, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   33B. The antibody-drug conjugate for use of embodiment 31B or 32B, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   34B. The antibody-drug conjugate for use of any one of embodiments 31B-33B, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   35B. The antibody-drug conjugate for use of any one of embodiments 31B-34B, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   36B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   37B. The antibody-drug conjugate for use of embodiment 36B, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   38B. The antibody-drug conjugate for use of embodiment 36B or 37B, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   39B. The antibody-drug conjugate for use of any one of embodiments 36B-38B, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   40B. The antibody-drug conjugate for use of any one of embodiments 36B-39B, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   41B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   42B. The antibody-drug conjugate for use of embodiment 41B, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   43B. The antibody-drug conjugate for use of embodiment 41B or 42B, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   44B. The antibody-drug conjugate for use of any one of embodiments 41B-43B, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   45B. The antibody-drug conjugate for use of any one of embodiments 41B-44B, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   46B. An antibody-drug conjugate that binds to tissue factor (TF) for use in the treatment of cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   47B. The antibody-drug conjugate for use of embodiment 46B, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   48B. The antibody-drug conjugate for use of embodiment 46B or 47B, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   49B. The antibody-drug conjugate for use of any one of embodiments 46B-48B, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   50B. The antibody-drug conjugate for use of any one of embodiments 46B-49B, wherein the objective response rate is at least about between about 13% and about 35%, optionally, wherein the objective response rate is at least about 14%, about 19%, about 21%, 23.8%, about 24%, about 25%, about 26%, about 28%, about 30%, or about 33%.
   51B. The antibody-drug conjugate for use of any one of embodiments 26B-50B, wherein the subject has been previously treated with bevacizumab.
   52B. The antibody-drug conjugate for use of any one of embodiments 26B-50B, wherein the subject has not been previously treated with bevacizumab.
   53B. The antibody-drug conjugate for use of any one of embodiments 26B-52B, wherein the subject has experienced disease progression during or after treatment with:
a) paclitaxel and cisplatin,
b) paclitaxel and carboplatin,
c) paclitaxel and topotecan,
d) bevacizumab, paclitaxel, and cisplatin,
e) bevacizumab, paclitaxel, and carboplatin, or
f) bevacizumab, paclitaxel, and topotecan.
   54B. The antibody-drug conjugate for use of any one of embodiments 26B-53B, wherein the subject is less than 65 years old.
   55B. The antibody-drug conjugate for use of any one of embodiments 26B-54B, wherein the subject has an ECOG score of 0.
   56B. The antibody-drug conjugate for use of any one of embodiments 26B-54B, wherein the subject has an ECOG score of 1.
   57B. The antibody-drug conjugate for use of any one of embodiments 1B-56B, wherein the cervical cancer is an adenocarcinoma.
   58B. The antibody-drug conjugate for use of any one of embodiments 1B-56B, wherein the cervical cancer is an adenosquamous carcinoma.
   59B. The antibody-drug conjugate for use of any one of embodiments 1B-56B, wherein the cervical cancer is a squamous cell carcinoma.
   60B. The antibody-drug conjugate for use of any one of embodiments 1B-56B, wherein the cervical cancer is a non-squamous cell carcinoma.
   61B. The antibody-drug conjugate for use of any one of embodiments 1B-60B, wherein the dose is about 2.0 mg/kg.
   62B. The antibody-drug conjugate for use of any one of embodiments 1B-60B, wherein the dose is 2.0 mg/kg.
   63B. The antibody-drug conjugate for use of any one of embodiments 1B-62B, wherein the antibody-drug conjugate is administered once about every 1 week, 2 weeks, 3 weeks or 4 weeks.
   64B. The antibody-drug conjugate for use of any one of embodiments 1B-62B, wherein the antibody-drug conjugate is administered once about every 3 weeks.
   65B. The antibody-drug conjugate for use of any one of embodiments 1B-64B, wherein the antibody-drug conjugate is administered once every 3 weeks.
   66B. The antibody-drug conjugate for use of any one of embodiments 1B-65B, wherein the cervical cancer is recurrent or metastatic cervical cancer.
   67B. The antibody-drug conjugate for use of any one of embodiments 1B-66B, wherein the subject has been previously treated with one or more therapeutic agents and did not respond to the treatment, wherein the one or more therapeutic agents is not the antibody-drug conjugate.
   68B. The antibody-drug conjugate for use of any one of embodiments 1B-66B, wherein the subject has been previously treated with one or more therapeutic agents and relapsed after the treatment, wherein the one or more therapeutic agents is not the antibody-drug conjugate.
   69B. The antibody-drug conjugate for use of any one of embodiments 1B-66B, wherein the subject has been previously treated with one or more therapeutic agents and has experienced disease progression during treatment, wherein the one or more therapeutic agents is not the antibody-drug conjugate.
   70B. The antibody-drug conjugate for use of any one of embodiments 67B-69B, wherein the one or more therapeutic agents is a platinum-based therapeutic agent.
   71B. The antibody-drug conjugate for use of any one of embodiments 67B-69B, wherein the one or more therapeutic agents is selected from the group consisting of: paclitaxel, cisplatin, carboplatin, topotecan, gemcitabine, fluorouracil, ixabepilone, imatinib mesylate, docetaxel, gefitinib, nab-paclitaxel, pemetrexed, vinorelbine, doxil, cetuximab, pembrolizumab, nivolumab and bevacizumab.
   72B. The antibody-drug conjugate for use of any one of embodiments 1B-71B, wherein the subject is not a candidate for curative therapy.
   73B. The antibody-drug conjugate for use of embodiment 72B, wherein the curative therapy comprises radiotherapy and/or exenterative surgery.
   74B. The antibody-drug conjugate for use of any one of embodiments 1B-71B, wherein the subject has received prior radiation to the pelvis.
   75B. The antibody-drug conjugate for use of any one of embodiments 1B-71B, wherein the subject has not received prior radiation to the pelvis.
   76B. The antibody-drug conjugate for use of any one of embodiments 1B-75B, wherein the subject has received one prior line of systemic therapy for relapsed, recurrent or metastatic cancer.
   77B. The antibody-drug conjugate for use of any one of embodiments 1B-75B, wherein the subject has received two prior lines of systemic therapy for relapsed, recurrent or metastatic cancer.
   78B. The antibody-drug conjugate for use of any one of embodiments 76B or 77B, wherein the subject did not respond to treatment with the prior systemic therapy.
   79B. The antibody-drug conjugate for use of any one of embodiments 76B or 77B, wherein the subject relapsed after treatment with the prior systemic therapy.
   80B. The antibody-drug conjugate for use of any one of embodiments 1B-79B, wherein the cervical cancer is an advanced stage cervical cancer, such as a stage 3 or stage 4 cervical cancer, such as metastatic cervical cancer.
   81B. The antibody-drug conjugate for use of any one of embodiments 1B-80B, wherein the cervical cancer is recurrent cervical cancer.
   82B. The antibody-drug conjugate for use of any one of embodiments 1B-81B, wherein the monomethyl auristatin is monomethyl auristatin E (MMAE).
   83B. The antibody-drug conjugate for use of any one of embodiments 1B-82B, wherein the anti-TF antibody or antigen-binding fragment thereof of the antibody-drug conjugate is a monoclonal antibody or a monoclonal antigen-binding fragment thereof.
   84B. The antibody-drug conjugate for use of any one of embodiments 1B-83B, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
   wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6, wherein the CDRs of the anti-TF antibody or antigen-binding fragment thereof of the antibody-drug conjugate are defined by the IMGT numbering scheme.
   85B. The antibody-drug conjugate for use of any one of embodiments 1B-84B, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
   86B. The antibody-drug conjugate for use of any one of embodiments 1B-85B, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
   87B. The antibody-drug conjugate for use of any one of embodiments 1B-86B, wherein the anti-TF antibody of the antibody-drug conjugate is tisotumab.
   88B. The antibody-drug conjugate for use of any one of embodiments 1B-87B, wherein the antibody-drug conjugate further comprises a linker between the anti-TF antibody or antigen-binding fragment thereof and the monomethyl auristatin. 89B. The antibody-drug conjugate for use of embodiment 88B, wherein the linker is a cleavable peptide linker. 90B. The antibody-drug conjugate for use of embodiment 89B, wherein the cleavable peptide linker has a formula: -MC-vc-PAB-, wherein:
a) MC is:

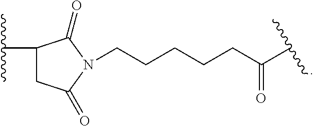 [SMILES: *C(=O)CCCCCN1C(=O)CC(*)C1=O]

b) vc is the dipeptide valine-citrulline, and
c) PAB is:

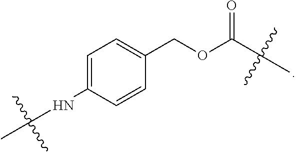 [SMILES: *Nc1ccc(COC(*)=O)cc1]

91B. The antibody-drug conjugate for use of any one of embodiments 88B-90B, wherein the linker is attached to sulphydryl residues of the anti-TF antibody obtained by partial reduction or full reduction of the anti-TF antibody or antigen-binding fragment thereof.
92B. The antibody-drug conjugate for use of embodiment 91B, wherein the linker is attached to MMAE, wherein the antibody-drug conjugate has the following-structure:

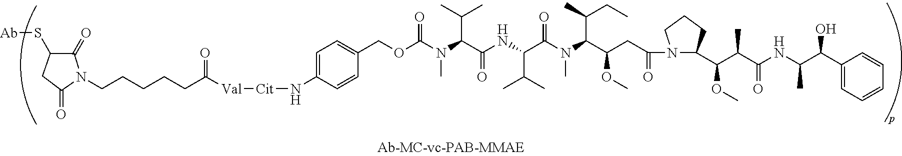

wherein p denotes a number from 1 to 8, S represents a sulphydryl residue of the anti-TF antibody, and Ab designates the anti-TF antibody or antigen-binding fragment thereof.
93B. The antibody-drug conjugate for use of embodiment 92B, wherein the average value of p in a population of the antibody-drug conjugates is about 4.
94B. The antibody-drug conjugate for use of any one of embodiments 1B-93B, wherein the antibody-drug conjugate is tisotumab vedotin or a biosimilar thereof.
95B. The antibody-drug conjugate for use of any one of embodiments 1B-94B, wherein the route of administration for the antibody-drug conjugate is intravenous. 96B. The antibody-drug conjugate for use of any one of embodiments 1B-95B, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF.
97B. The antibody-drug conjugate for use of any one of embodiments 1B-96B, wherein the subject has a TF histology score (H-score) of at least 1.
98B. The antibody-drug conjugate for use of any one of embodiments 1B-97B, wherein the subject has one or more adverse events and is further administered an additional therapeutic agent to eliminate or reduce the severity of the one or more adverse events.
99B. The antibody-drug conjugate for use of any one of embodiments 1B-97B, wherein the subject is at risk of developing one or more adverse events and is further administered an additional therapeutic agent to prevent or reduce the severity of the one or more adverse events.
100B. The antibody-drug conjugate for use of anyone of embodiments 1B-98B, wherein the subject has one or more adverse events and the dose of the antibody drug conjugate is reduced following the one or more adverse events.
101B. The antibody-drug conjugate for use of embodiment 100B, wherein the dose is reduced from 2.0 mg/kg to 1.3 mg/kg.

102B. The antibody-drug conjugate for use of embodiment 100B or 101B, wherein the dose is reduced from 1.3 mg/kg to 0.9 mg/kg.

103B. The antibody-drug conjugate for use of any one of embodiments 98B-102B, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.

104B. The antibody-drug conjugate for use of any one of embodiments 98B-103B, wherein the one or more adverse events is a grade 3 or greater adverse event.
105B. The antibody-drug conjugate for use of any one of embodiments 98B-103B, wherein the one or more adverse events is a serious adverse event.
106B. The antibody-drug conjugate for use of embodiment 98B or embodiment 99B, wherein the one or more adverse events is conjunctivitis and/or keratitis and the additional agent is a preservative-free lubricating eye drop, an ocular vasoconstrictor and/or a steroid eye drop.
107B. The antibody-drug conjugate for use of any one of embodiments 1B-106B, wherein the antibody-drug conjugate is administered as a monotherapy.
108B. The antibody-drug conjugate for use of any one of embodiments 1B-107B, wherein the subject is a human.
109B. The antibody-drug conjugate for use of any one of embodiments 1B-108B, wherein the antibody-drug conjugate is in a pharmaceutical composition comprising the antibody-drug conjugate and a pharmaceutical acceptable carrier.

### Use for the Manufacture of a Medicament

1C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has been previously treated with bevacizumab.
2C. The use of embodiment 1C, wherein the subject has an ECOG score of 0.
3C. The use of embodiment 1C, wherein the subject has an ECOG score of 1.
4C. The use of any one of embodiments 1C-3C, wherein the subject is less than 65 years old.
5C. The use of any one of embodiments 1C-4C, wherein the subject has been previously treated with:
a) paclitaxel and cisplatin,
b) paclitaxel and carboplatin, or
c) paclitaxel and topotecan.
   6C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has not been previously treated with bevacizumab.
   7C. The use of embodiment 6C, wherein the subject has an ECOG score of 0.
   8C. The use of embodiment 6C, wherein the subject has an ECOG score of 1.
   9C. The use of any one of embodiments 6C-8C, wherein the subject is less than 65 years old.
   10C. The use of any one of embodiments 6C-9C, wherein the subject has been previously treated with:
a) paclitaxel and cisplatin,
b) paclitaxel and carboplatin, or
c) paclitaxel and topotecan.
   11C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has an Eastern Cooperative Oncology Group (ECOG) score of 0.
   12C. The use of embodiment 11C, wherein the subject is less than 65 years old.
   13C. The use of any embodiment 11C or 12C, wherein the subject has been previously treated with:
a) paclitaxel and cisplatin,
b) paclitaxel and carboplatin, or
c) paclitaxel and topotecan.
   14C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject has an ECOG score of 1.
   15C. The use of embodiment 14C, wherein the subject is less than 65 years old.
   16C. The use of any embodiment 14C or 15C, wherein the subject has been previously treated with:
a) paclitaxel and cisplatin,
b) paclitaxel and carboplatin, or
c) paclitaxel and topotecan.
   17C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject is less than 65 years old.
   18C. The use of embodiment 17C, wherein the subject has an ECOG score of 0.
   19C. The use of embodiment 17C, wherein the subject has an ECOG score of 1.
   20C. The use of any one of embodiments 17C-19C, wherein the subject has been previously treated with:
a) paclitaxel and cisplatin,
b) paclitaxel and carboplatin, or
c) paclitaxel and topotecan.
   21C. The use of any one of embodiments 1C-20C, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   22C. The use of any one of embodiments 1C-21C, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   23C. The use of any one of embodiments 1C-22C, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   24C. The use of any one of embodiments 1C-23C, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   25C. The use of any one of embodiments 1C-24C, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   26C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   27C. The use of embodiment 26C, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   28C. The use of embodiment 26C or 27C, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   29C. The use of any one of embodiments 26C-28C, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   30C. The use of any one of embodiments 26C-29C, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   31C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   32C. The use of embodiment 31C, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   33C. The use of embodiment 31C or 32C, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   34C. The use of any one of embodiments 31C-33C, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   35C. The use of any one of embodiments 31C-34C, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   36C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   37C. The use of embodiment 36C, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   38C. The use of embodiment 36C or 37C, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   39C. The use of any one of embodiments 36C-38C, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   40C. The use of any one of embodiments 36C-39C, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   41C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   42C. The use of embodiment 41C, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   43C. The use of embodiment 41C or 42C, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   44C. The use of any one of embodiments 41C-43C, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.
   45C. The use of any one of embodiments 41C-44C, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   46C. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 0.9 mg/kg to about 2.1 mg/kg, wherein the time to response is less than about 6 months after administration of the antibody-drug conjugate, optionally, wherein the time to response is less than about 4 months, about 2 months, 1.4 months, or about 1.2 months after administration of the antibody-drug conjugate.
   47C. The use of embodiment 46C, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.
   48C. The use of embodiment 46C or 47C, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.
   49C. The use of any one of embodiments 46C-48C, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.
   50C. The use of any one of embodiments 46C-49C, wherein the objective response rate is at least about between about 13% and about 35%, optionally, wherein the objective response rate is at least about 14%, about 19%, about 21%, 23.8%, about 24%, about 25%, about 26%, about 28%, about 30%, or about 33%.
   51C. The use of any one of embodiments 26C-50C, wherein the subject has been previously treated with bevacizumab.
   52C. The use of any one of embodiments 26C-50C, wherein the subject has not been previously treated with bevacizumab.
   53C. The use of any one of embodiments 26C-52C, wherein the subject has experienced disease progression during or after treatment with:
a) paclitaxel and cisplatin,
b) paclitaxel and carboplatin,
c) paclitaxel and topotecan,
d) bevacizumab, paclitaxel, and cisplatin,
e) bevacizumab, paclitaxel, and carboplatin, or
f) bevacizumab, paclitaxel, and topotecan.
   54C. The use of any one of embodiments 26C-53C, wherein the subject is less than 65 years old.
   55C. The use of any one of embodiments 26C-54C, wherein the subject has an ECOG score of 0.
   56C. The use of any one of embodiments 26C-54C, wherein the subject has an ECOG score of 1.
   57C. The use of any one of embodiments 1C-56C, wherein the cervical cancer is an adenocarcinoma.
   58C. The use of any one of embodiments 1C-56C, wherein the cervical cancer is an adenosquamous carcinoma.
   59C. The use of any one of embodiments 1C-56C, wherein the cervical cancer is a squamous cell carcinoma.
   60C. The use of any one of embodiments 1C-56C, wherein the cervical cancer is a non-squamous cell carcinoma.
   61C. The use of any one of embodiments 1C-60C, wherein the dose is about 2.0 mg/kg.
   62C. The use of any one of embodiments 1C-60C, wherein the dose is 2.0 mg/kg.
   63C. The use of any one of embodiments 1C-62C, wherein the antibody-drug conjugate is administered once about every 1 week, 2 weeks, 3 weeks or 4 weeks.
   64C. The use of any one of embodiments 1C-62C, wherein the antibody-drug conjugate is administered once about every 3 weeks.
   65C. The use of any one of embodiments 1C-64C, wherein the antibody-drug conjugate is administered once every 3 weeks.
   66C. The use of any one of embodiments 1C-65C, wherein the cervical cancer is recurrent or metastatic cervical cancer.
   67C. The use of any one of embodiments 1C-66C, wherein the subject has been previously treated with one or more therapeutic agents and did not respond to the treatment, wherein the one or more therapeutic agents is not the antibody-drug conjugate.
   68C. The use of any one of embodiments 1C-67C, wherein the subject has been previously treated with one or more therapeutic agents and relapsed after the treatment, wherein the one or more therapeutic agents is not the antibody-drug conjugate.
   69C. The use of any one of embodiments 1C-68C, wherein the subject has been previously treated with one or more therapeutic agents and has experienced disease progression during treatment, wherein the one or more therapeutic agents is not the antibody-drug conjugate.
   70C. The use of any one of embodiments 67C-69C, wherein the one or more therapeutic agents is a platinum-based therapeutic agent.
   71C. The use of any one of embodiments 67C-69C, wherein the one or more therapeutic agents is selected from the group consisting of: paclitaxel, cisplatin, carboplatin, topotecan, gemcitabine, fluorouracil, ixabepilone, imatinib mesylate, docetaxel, gefitinib, nab-paclitaxel, pemetrexed, vinorelbine, doxil, cetuximab, pembrolizumab, nivolumab and bevacizumab.
   72C. The use of any one of embodiments 1C-71C, wherein the subject is not a candidate for curative therapy.
   73C. The use of embodiment 72C, wherein the curative therapy comprises radiotherapy and/or exenterative surgery.
   74C. The use of any one of embodiments 1C-71C, wherein the subject has received prior radiation to the pelvis.
   75C. The use of any one of embodiments 1C-71C, wherein the subject has not received prior radiation to the pelvis.
   76C. The use of any one of embodiments 1C-75C, wherein the subject has received one prior line of systemic therapy for relapsed, recurrent or metastatic cancer.
   77C. The use of any one of embodiments 1C-75C, wherein the subject has received two prior lines of systemic therapy for relapsed, recurrent or metastatic cancer.
   78C. The use of any one of embodiments 76C or 77C, wherein the subject did not respond to treatment with the prior systemic therapy.

79C. The use of any one of embodiments 76C or 77C, wherein the subject relapsed after treatment with the prior systemic therapy.

80C. The use of any one of embodiments 1C-79C, wherein the cervical cancer is an advanced stage cervical cancer, such as a stage 3 or stage 4 cervical cancer, such as metastatic cervical cancer.
81C. The use of any one of embodiments 1C-80C, wherein the cervical cancer is recurrent cervical cancer.
82C. The use of any one of embodiments 1C-81C, wherein the monomethyl auristatin is monomethyl auristatin E (MMAE).
83C. The use of any one of embodiments 1C-82C, wherein the anti-TF antibody or antigen-binding fragment thereof of the antibody-drug conjugate is a monoclonal antibody or a monoclonal antigen-binding fragment thereof.
84C. The use of any one of embodiments 1C-83C, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
   wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6, wherein the CDRs of the anti-TF antibody or antigen-binding fragment thereof of the antibody-drug conjugate are defined by the IMGT numbering scheme.
   85C. The use of any one of embodiments 1C-84C, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
   86C. The use of any one of embodiments 1C-85C, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO: 8.
   87C. The use of any one of embodiments 1C-86C, wherein the anti-TF antibody of the antibody-drug conjugate is tisotumab.
   88C. The use of any one of embodiments 1C-87C, wherein the antibody-drug conjugate further comprises a linker between the anti-TF antibody or antigen-binding fragment thereof and the monomethyl auristatin.
   89C. The use of embodiment 88C, wherein the linker is a cleavable peptide linker.
   90C. The use of embodiment 89C, wherein the cleavable peptide linker has a formula: -MC-vc-PAB-, wherein:
a) MC is:

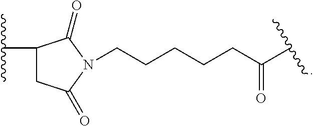 [SMILES: *C(=O)CCCCCN1C(=O)CC(*)C1=O]

b) vc is the dipeptide valine-citrulline, and
c) PAB is:

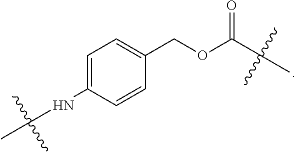 [SMILES: *Nc1ccc(COC(*)=O)cc1]

91C. The use of any one of embodiments 88C-90C, wherein the linker is attached to sulphydryl residues of the anti-TF antibody obtained by partial reduction or full reduction of the anti-TF antibody or antigen-binding fragment thereof.
92C. The use of embodiment 91C, wherein the linker is attached to MMAE, wherein the antibody-drug conjugate has the following-structure:

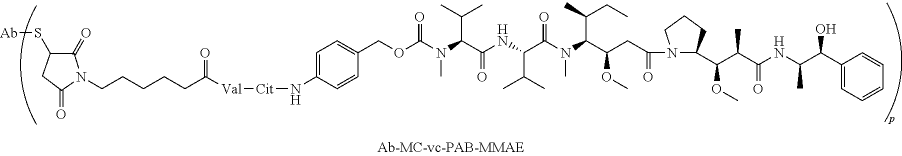

wherein p denotes a number from 1 to 8, S represents a sulphydryl residue of the anti-TF antibody, and Ab designates the anti-TF antibody or antigen-binding fragment thereof.
93C. The use of embodiment 92C, wherein the average value of p in a population of the antibody-drug conjugates is about 4.
94C. The use of any one of embodiments 1C-93C, wherein the antibody-drug conjugate is tisotumab vedotin or a biosimilar thereof.
95C. The use of any one of embodiments 1C-94C, wherein the route of administration for the antibody-drug conjugate is intravenous.
96C. The use of any one of embodiments 1C-95C, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF.
97C. The use of any one of embodiments 1C-96C, wherein the subject has a TF histology score (H-score) of at least 1.
98C. The use of any one of embodiments 1C-97C, wherein the subject has one or more adverse events and is further administered an additional therapeutic agent to eliminate or reduce the severity of the one or more adverse events.
99C. The use of any one of embodiments 1C-97C, wherein the subject is at risk of developing one or more adverse events and is further administered an additional therapeutic agent to prevent or reduce the severity of the one or more adverse events.
100C. The use of any one of embodiments 1C-98C, wherein the subject has one or more adverse events and the dose of the antibody drug conjugate is reduced following the one or more adverse events.
101C. The use of embodiment 100C, wherein the dose is reduced from 2.0 mg/kg to 1.3 mg/kg.
102C. The use of embodiment 100C or 101C, wherein the dose is reduced from 1.3 mg/kg to 0.9 mg/kg.
103C. The use of any one of embodiments 98C-102C, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.
104C. The use of any one of embodiments 98C-103C, wherein the one or more adverse events is a grade 3 or greater adverse event.
105C. The use of any one of embodiments 98C-103C, wherein the one or more adverse events is a serious adverse event.
106C. The use of embodiment 98C or embodiment 99C, wherein the one or more adverse events is conjunctivitis and/or keratitis and the additional agent is a preservative-free lubricating eye drop, an ocular vasoconstrictor and/or a steroid eye drop.
107C. The use of any one of embodiments 1C-106C, wherein the antibody-drug conjugate is administered as a monotherapy.
108C. The use of any one of embodiments 1C-107C, wherein the subject is a human.
109C. The use of any one of embodiments 1C-108C, wherein the antibody-drug conjugate is in a pharmaceutical composition comprising the antibody-drug conjugate and a pharmaceutical acceptable carrier.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: A Phase II Trial of Tisotumab Vedotin in Subjects with Previously Treated, Recurrent or Metastatic Cervical Cancer

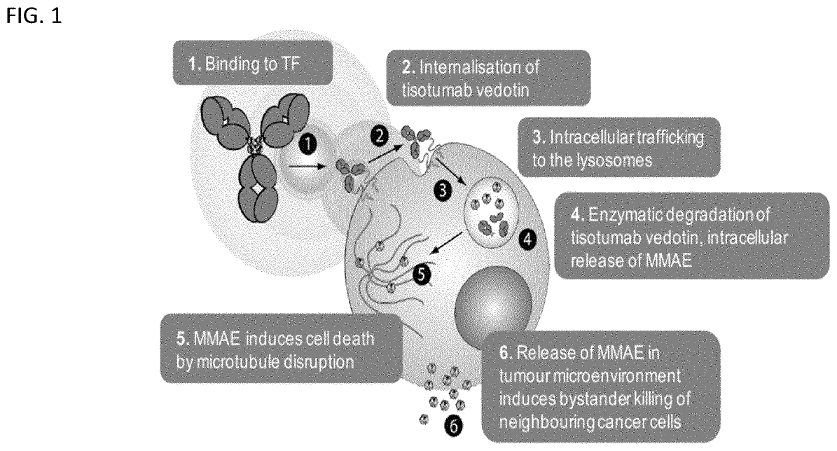

Tisotumab vedotin is an antibody-drug conjugate comprising an antibody that binds to tissue factor (TF), a protease-cleavable linker, and the microtubule disrupting agent MMAE. TF is a protein aberrantly expressed in a wide number of tumors including cervical cancer and is associated with poor prognosis. See Forster Y et al. Clin Chim Acta. 2006; 364(1-2):12-21 and Cocco E et al. BMC Cancer. 2011; 11:263. Tisotumab vedotin selectively targets TF to deliver a clinically validated toxic payload to tumor cells (FIG. 1). See Breij E C et al. Cancer Res. 2014; 74(4):1214-1226 and Chu A J. Int J Inflam. 2011; 2011. doi: 10.4061/2011/367284.

The efficacy, safety and tolerability of 2.0 mg/kg tisotumab vedotin in patients with previously treated, advanced cervical cancer (e.g., recurrent and/or metastatic cancer) was evaluated. Preliminary data observed in a cohort of previously treated cervical cancer patients suggest a positive benefit risk profile for this population of high unmet need.

### Patients

Eligible patients had recurrent or extra-pelvic metastatic cervical cancer with squamous cell, adenocarcinoma or adenosquamous histology; progressive disease (PD) during or after doublet chemotherapy (paclitaxel-platinum or paclitaxel-topotecan) plus bevacizumab, if eligible; measurable disease according to Response Evaluation Criteria In Solid Tumors (RECIST) v1.1; and Eastern Cooperative Oncology Group (ECOG) performance status of 0 or 1. Patients who received >2 prior systemic treatment regimens for r/mCC or who received prior treatment with MMAE-derived drugs were excluded. Patients on anticoagulation therapy were allowed on the study if their activated partial thromboplastin time test was ≤1.25 upper limit of normal, were not on concurrent prophylactic acetylsalicylic acid, and if on anticoagulants that required laboratory assessments for dose titration, were on a steady dose for ≥4 weeks prior to the first TV administration with an international normalized ratio ≤2.5. Patients with neuroendocrine or sarcomatoid tumor histology, known coagulation defects leading to an increased risk of bleeding, diffuse alveolar hemorrhage from vasculitis, known bleeding diathesis, ongoing major bleeding, trauma with increased risk of life-threatening bleeding, history of severe head trauma, intracranial surgery within 8 weeks of study entry, active ocular surface disease, prior episodes of cicatricial conjunctivitis or Steven Johnson syndrome, and Common Toxicity Criteria for Adverse Events (CTCAE) grade ≥2 neuropathy were excluded

### Methods

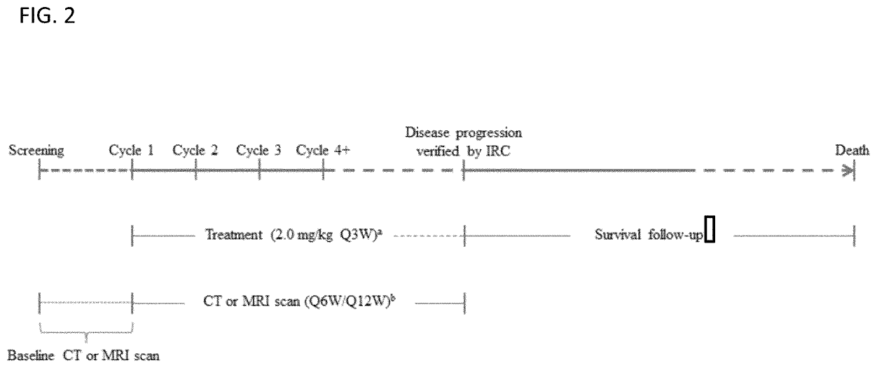

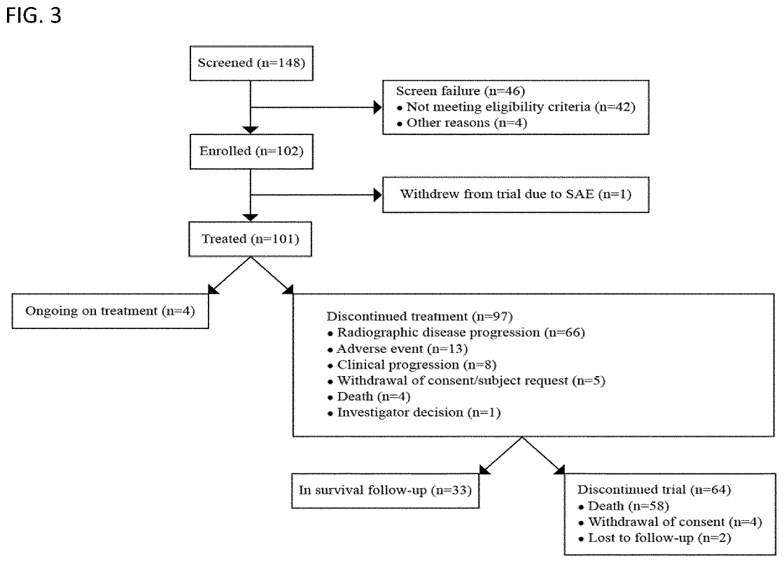

This phase II single arm, multicenter, international trial evaluated the efficacy, safety and tolerability of 2.0 mg/kg tisotumab vedotin in patients with recurrent or metastatic cervical cancer. Eligible patients had experienced disease progression during or after treatment with a chemotherapy doublet in combination with bevacizumab if eligible to receive bevacizumab. Patients had received no more than 2 prior systemic therapies for their metastatic or recurrent disease. Eligible patients were treated with intravenous (IV) tisotumab vedotin 2.0 mg/kg, every 3 weeks (1Q3W) until they met a predefined discontinuation criterion (FIG. 2). Imaging was obtained every six weeks for the first 30 weeks and every 12 weeks thereafter. Responses were confirmed no earlier than 4 weeks (28 days) after the first assessment of response. 102 patients, age ≥18 years, were enrolled into the trial and 101 patients were subsequently treated (FIG. 3).

Inclusion criteria and exclusion criteria for patients enrolled in trial are shown in Table 1.

TABLE 1
List of inclusion and exclusion criteria

| | |
|---|---|
| Inclusion Criteria | Patients with extra-pelvic metastatic or recurrent cervical cancer |
| | including squamous cell, adenocarcinoma or adenosquamous |
| | histology, that: |
| | Have experienced disease progression during or after |
| | treatment with: |
| | chemotherapy doublet including paclitaxel and cisplatin or |
| | carboplatin |
| | OR |
| | paclitaxel and topotecan, |
| | and who have received or are ineligible for treatment with |
| | bevacizumab according to local standards. |
| | Have received no more than 2 prior systemic treatment |
| | regimens for recurrent or metastatic cervical cancer. |
| | Are not candidates for curative therapy, including but not |
| | limited to, radiotherapy or exenterative surgery. |
| | Measurable disease according to RECIST vl.l as assessed by |
| | independent central imaging review. |
| | Age ≥18 years. |
| | Acceptable renal function: Calculated (Cockcroft-Gault) |
| | Glomerular Filtration Rate (GFR) >45 mL/min. |
| | Acceptable liver function: Alanine aminotransferase (ALT) and |
| | aspartate aminotransferase (AST) ≤3 times the upper limit of |
| | normal (ULN) (if liver tumor/metastases are present, then ≤5× |
| | ULN is allowed); bilirubin ≤1.5× ULN, except in patients |
| | diagnosed with Gilbert's syndrome, direct bilirubin ≤2× ULN. |
| | Acceptable hematological status: Hemoglobin ≥5.6 mmol/L |
| | (9.0 g/dL), absolute neutrophil count (ANC) ≥500/μL |
| | (1.5 × 10⁹/L); platelet count ≥100 × 10⁹/L assessed at least 2 weeks |
| | after transfusion with blood products and/or growth factor |
| | support. |
| | Acceptable coagulation status: International normalized ratio |
| | (INR) ≤1.2 (patients not on anti-coagulation therapy), and |
| | activated partial thromboplastin time (aPTT) ≤1.25 ULN; patients |
| | on anti-coagulation therapy (e.g., warfarin) must be on a steady |
| | dose (no active titration) for at least 4 weeks prior to screening and |
| | must have an INR ≤2.5 for eligibility. |
| | Eastern Cooperative Oncology Group (ECOG) performance |
| | status of 0 or 1 assessed within 7 days of cycle 1 day 1. |
| | Life expectancy of at least three months. |
| | A negative serum pregnancy test (in patients 18-55 years of age; |
| | post-menopause must be confirmed in eCRF for patients >55 |
| | years). Women who are pregnant or breast feeding are ineligible. |
| | Patients of reproductive potential must agree to use adequate |
| | contraception during and for 6 months after the last administration |
| | of tisotumab vedotin. Adequate contraception for women is |
| | defined as highly effective methods of contraception. In countries |
| | where two highly effective methods of contraception are required |
| | this will be an inclusion criterion. |
| | All patients must provide biopsy specimen during screening. |
| | Archival or fresh core biopsies are required (aspirates are not |
| | acceptable). FFPE blocks OR at least 10 slides with 5 micron thick |
| | sections are acceptable for eligibility. |
| | Following receipt of verbal and written information about the trial, |
| | patients must provide signed informed consent before any trial- |
| | related activity is carried out. |
| Exclusion Criteria | Hematological: Known past or current coagulation defects leading |
| | to an increased risk of bleeding; diffuse alveolar hemorrhage from |
| | vasculitis; known bleeding diathesis; ongoing major bleeding; |
| | trauma with increased risk of life-threatening bleeding or history |
| | of severe head trauma or intracranial surgery within two months |
| | of trial entry. |
| | Cardiovascular: Clinically significant cardiac disease including |
| | unstable angina, acute myocardial infarction 6 months prior to |
| | screening; known congestive heart failure (Grade I or IV as |
| | classified by the New York Heart Association), and/or a known |
| | decreased cardiac ejection fraction of <45%; a marked baseline |
| | prolongation of QT/QTc interval (e.g., repeated demonstration of |
| | a QTc interval >450 msec), a complete left bundle branch block |
| | (defined as a QRS interval ≥120 msec in left bundle branch block |
| | form) or an incomplete left bundle branch block. |
| | Central nervous system: Any history of intracerebral |
| | arteriovenous malformation, cerebral aneurysm, or stroke |
| | (transient ischemic attack >1 month prior to screening is |
| | allowed). |
| | Ophthalmological: Active ocular surface disease at baseline (as |
| | evaluated by ophthalmologist in case active ocular surface disease |
| | is suspected by the investigator). Patients with any prior episode |
| | of cicatricial conjunctivitis or Steven Johnson syndrome (as |
| | evaluated by the investigator) are ineligible. |
| | Other cancer/metastases: Known past or current malignancy other |
| | than inclusion diagnosis, except for: non-invasive basal cell or |
| | squamous cell skin carcinoma; noninvasive, superficial bladder |
| | cancer; any curable cancer with a complete response (CR) |
| | of >5 years duration. |
| | Brain metastases are allowed if the following criteria are met: |
| | Definitive therapy (for example: surgery or stereotactic brain |
| | radiotherapy) has been completed >28 days before the first |
| | dose of tisotumab vedotin; the patient has no evidence of |
| | clinical or radiologic tumor progression; patients have |
| | completed perioperative corticosteroid therapy or steroid |
| | taper. Chronic steroid therapy is acceptable provided that the |
| | dose is stable for 1 month prior to screening. |
| | Excluded medications or treatment regimens: Therapeutic anti- |
| | coagulation therapy or anti-platelet therapy UNLESS the patient |
| | is no longer being actively titrated for their anti-coagulation (e.g. |
| | warfarin) and is on steady doses for at least 4 weeks prior to |
| | screening; cumulative dose of corticosteroid ≥150 mg |
| | (prednisone or equivalent doses of corticosteroids) within 2 weeks |
| | of the first tisotumab vedotin administration. |
| | Surgery/procedures: Major surgery within 4 weeks or open biopsy |
| | within 7 days prior to the first tisotumab vedotin administration. |
| | Patients who have planned major surgery during the treatment |
| | period must be excluded from the trial. |
| | Peripheral neuropathy grade ≥2 |
| | Prior therapy: |
| | Any prior treatment with MMAE-derived drugs. |
| | Any anti-cancer therapy, including small molecules, |
| | immunotherapy, chemotherapy, monoclonal antibodies, or |
| | any other experimental drug within 28 days prior to first |
| | tisotumab vedotin administration. Patients, who have not |
| | recovered from symptomatic side effects of radiotherapy or |
| | symptoms of autoimmune toxicities related to prior immune |
| | therapy at the time of initiation of screening procedure, are not |
| | eligible. |
| | Other: Ongoing significant, uncontrolled medical condition; |
| | clinically significant active viral, bacterial or fungal infection |
| | requiring IV or oral (PO) treatment with antimicrobial therapy |
| | ending less than 7 days prior to first tisotumab vedotin |
| | administration; known human immunodeficiency virus |
| | seropositivity; known history of hepatitis B or C infection. |
| | Patient has any condition for which, in the opinion of the |
| | investigator, participation would not be in the best interest of the |
| | patient (e.g. compromise the well-being) or that could prevent, |
| | limit, or confound the protocol-specified assessments. |
| | Patient has known allergies, hypersensitivity, or intolerance to |
| | tisotumab vedotin or its excipients. |

Lyophilized vials containing 40 mg of tisotumab vedotin were stored in a refrigerator at 2° C. to 8° C. Tisotumab vedotin was reconstituted in 4 ml of water leading to a reconstituted solution comprising 10 mg/mL tisotumab vedotin, 30 mM histidine, 88 mM sucrose, and 165 mM D-mannitol. The reconstituted antibody drug-conjugate solution had a pH of 6.0. The reconstituted tisotumab vedotin was diluted into a 0.9% NaCl 100 mL infusion bag according to the dose calculated for the patient to receive 2.0 mg/kg tisotumab vedotin. Intravenous infusion was completed within 24 hours after the tisotumab vedotin vial was reconstituted. A 0.2 μm in-line filter was used for the intravenous infusion. The entire 100 mL volume from the prepared infusion bag was administered. No dead volume was provided. For patients that did not tolerate the protocol-specified dosing schedule, dose reductions were permitted in order to allow the patient to continue treatment with tisotumab vedotin (Table 2).

TABLE 2
Dose Modification Scheme

| Previous dose of tisotumab vedotin | Reduced dose of tisotumab vedotin |
|---|---|
| 2.0 mg/kg | 1.3 mg/kg |
| 1.3 mg/kg | 0.9 mg/kg |
| 0.9 mg/kg | 0.9 mg/kg* |

*If the patient was already being treated with tisotumab vedotin 0.9 mg/kg 1Q3W, the dose of tisotumab vedotin is not reduced further.

Objectives and endpoints are described in Table 3. The confirmed objective response rate (ORR) and a 2-sided 95% exact confidence interval was calculated 27 weeks after the last patient had received the first dose of tisotumab vedotin. Assuming a true confirmed ORR of 25% for tisotumab vedotin, 100 patients provides 96% power to exclude an ORR of 11% or less (one-sided P-value of 2.5%).

TABLE 3
Objectives and endpoints

| | |
|---|---|
| OBJECTIVES | ENDPOINTS |

Primary

| | |
|---|---|
| Determine the anti-tumor | Confirmed objective response rate (ORR) |
| efficacy in patients with | based upon RECIST v1.1 assessed by the |
| cervical cancer. | independent review committee. |

Secondary

| | |
|---|---|
| Evaluate durability and | Duration of response (DOR). |
| time to response. | Time to response (TTR). |
| Evaluate other clinical | Confirmed ORR by RECIST v1.1, |
| outcomes. | investigator assessment. |
| | Progression free survival (PFS) by |
| | RECIST v1.1 byIRC. |
| | Overall survival (OS). |
| Assess safety and | Adverse events and safety laboratory |
| tolerability. | parameters. |
| | Pharmacokinetics (PK). |
| | Immunogenicity (Anti-Drug Antibodies |
| | [ADAs]) of tisotumab vedotin. |

Exploratory

| | |
|---|---|
| Assess biomarkers related | TF expression in pre-treatment and post- |
| to clinical response. | progression tumor biopsies, circulating |
| | TF, proteomic analyses and genetic |
| | variations. |
| Assess potential | Circulating TF and proteomic analyses. |
| pharmacodynamic biomarkers | |
| of tisotumab vedotin. | |
| Assess Health Related | HRQL relevant questionnaires. |
| Quality of Life (HRQL) in | |
| cervical cancer patients | |
| treated with tisotumab | |
| vedotin. | |

If a patient's trial treatment was discontinued before the end of the treatment regimen, this did not result in automatic withdrawal of the patient from the trial. A patient's trial treatment was discontinued if: radiographic disease progression was verified by independent committee review; safety stopping rules were fulfilled; unacceptable toxicity required treatment discontinuation; the investigator believed that for safety reasons (e.g., adverse event) it was in the best interest of the patient to stop treatment; pregnancy; patient choice; and/or a new anti-cancer therapy was initiated. When treatment was discontinued, investigators performed a safety follow-up visit. The safety follow-up visit was performed 15 days±5 days after the last dose of tisotumab vedotin and prior to initiation of new anti-cancer treatment and included most assessments performed at screening and response assessments. Upon treatment discontinuation, patients continued to be followed for post-treatment assessments until death or withdrawal from the trial. Safety stopping rules for discontinuation of treatment included the following in case of ocular toxicity: first recurrence of CTCAE grade ≥3 conjunctivitis (despite dose reduction); third recurrence of CTCAE grade ≤2 keratitis (despite dose reductions); first occurrence of CTCAE grade ≥3 keratitis; ophthalmological evaluation reveals conjunctival/corneal scarring; any grade of symblepharon; any grade of fluorescent patches or conjunctival ulceration that did not stabilize or improve after dose reduction; or any dose delay related to ocular toxicity exceeding 12 weeks. Safety stopping rules for discontinuation of treatment included the following in case of other adverse events besides ocular toxicity: second occurrence of a grade 3 infusion related reaction (despite pre-medication); first occurrence of a ≥grade 4 infusion related reaction; first occurrence of mucositis ≥grade 4; first occurrence of peripheral neuropathy ≥grade 4; any event of pulmonary or CNS hemorrhage ≥grade 2; or any event of hemorrhage ≥grade 3 for patients on anti-coagulation therapy.

Three adverse events of special interest were ocular adverse events, adverse events of peripheral neuropathy, and adverse events of bleeding. For ocular AEs: AEs of grade 1-2 conjunctivitis were frequently reported in relation to treatment with tisotumab vedotin. Severe cases (CTCAE ≥grade 3) of conjunctivitis and keratitis were observed, however implementation of a comprehensive mitigation plan and preventive measures substantially reduced both the frequency and severity of ocular adverse reactions. Mitigation strategies for the prevention of ocular AEs included the use of preservative-free lubricating eye drops from the first dose of TV until 30 days after the last dose, application of steroid eye drops given prior to the start of TV infusion and continued for 72 hours thereafter, administration of local ocular vasoconstrictor eye drops before TV infusion, use of cooling eye pads during infusion, and avoidance of contact lens use while on treatment. In order to prevent ocular AEs, all patients must adhere to the following ocular premedication and preventative eye therapy guidelines:
Administration of local ocular vasoconstrictor before infusion (brimonidine tartrate 0.2% eye drops or similar, 3 drops in each eye immediately prior to start of infusion; otherwise to be used in accordance with the product prescribing information). If the patient does not tolerate ocular vasoconstrictors due to adverse reactions, continued treatment with these may be stopped at the discretion of the investigator and following discussion with the sponsor's medical monitor.
Use of eye cooling pads during infusion, e.g., Cardinal Health cold packs, refrigerator-based THERA PEARL Eye Mask, or similar. To be applied 5 minutes prior to start of infusion in accordance with the instructions provided with the eye cooling pads. The cooling pads must remain on the patient's eyes during the entire 30-60 minute infusion and for as long as 30 minutes afterwards.
Application of steroid eye drops (dexamethasone 0.1% eye drops or equivalent) before and after each infusion for a total of 4 days. The first drops are to be given 24 hours prior to start of infusion. Continue treatment for 72 hours thereafter. Steroid eye drops should be administered as 1 drop in each eye, 3 times daily, or used in accordance with the product prescribing information.
Use of lubricating eye drops during the whole treatment phase of the trial (i.e., from first dose of TV until 30 days after the last dose of TV). Frequent use of lubricating eye drops as per standard of care for patients receiving chemotherapy is recommended. Lubricating eyedrops should be self-administered daily or as needed according to the package insert or prescribed instructions from the ophthalmologist.
It is recommended that patients not wear contact lenses while being treated with tisotumab vedotin from the first dose until 30 days after the last dose of study drug.

The ocular treatment guidelines are shown in Table 4.

TABLE 4
Ocular treatment guidelines

| | Treatment guideline |
|---|---|
| Ocular symptom | (The length of treatment is decided |
| (CTCAE grading) | by the local ophthalmologist) |
| Conjunctivitis | The local ophthalmologist prescribes frequent |
| grade 1 | dosing of preservative-free topical steroid |
| | drops. |
| Conjunctivitis | The local ophthalmologist prescribes frequent |
| grade 2 | dosing (every second hour) of preservative-free |
| | topical steroid drops in conjunction with |
| | preservative free antibiotic prophylaxis |
| | such as chloramphenicol. |
| Conjunctivitis | The local ophthalmologist prescribes frequent |
| grade 3 | dosing (every second hour) of preservative-free |
| | topical steroid drops in conjunction with |
| | preservative free antibiotic prophylaxis |
| | such as chloramphenicol. |
| Keratitis | The local ophthalmologist prescribes frequent |
| grade 1 | dosing of preservative-free topical steroid |
| | drops. |
| Keratitis | The local ophthalmologist prescribes frequent |
| grade 2 | dosing (every second hour) of preservative-free |
| | topical steroid drops in conjunction with |
| | preservative free antibiotic prophylaxis |
| | such as chloramphenicol. |
| Conjunctival | The local ophthalmologist prescribes frequent |
| ulceration: | dosing (every second hour) of preservative-free |
| Any grade | topical steroid drops in conjunction with |
| | preservative free antibiotic prophylaxis |
| | such as chloramphenicol. |

For AEs of peripheral neuropathy (including neuropathy peripheral; peripheral sensory neuropathy; peripheral motor neuropathy; polyneuropathy): Peripheral neuropathy is a well-known adverse reaction to treatment with chemotherapeutics (including cisplatin and taxanes) as well as MMAE-based ADCs and is frequently reported in relation to treatment with tisotumab vedotin. The majority of the reported cases are grade 1-2; however peripheral neuropathy is the leading cause of permanently discontinuation of tisotumab vedotin treatment. A mitigation plan, including dose reduction (see Table 2) and dose delay (i.e., hold dosing until event has improved to ≤grade 1), was in place to prevent onset of peripheral neuropathy as well as deterioration of pre-existing conditions. For AEs of bleeding: Bleeding events were considered of special interest due to the mode of action of tisotumab vedotin. In line with preclinical findings, no major impact on activated partial thromboplastin time (aPTT) or prothrombin time (PT) has until now been found for tisotumab vedotin treated patients. Epistaxis was the most common reported AE, however, nearly all of the cases are grade 1. Excluding epistaxis, no causal relation has been established for the majority of the reported bleeding events and treatment with tisotumab vedotin.

Tumor biopsies were required prior to first TV administration, and an optional tumor biopsy was requested after IRC-assessed PD. Fresh pretreatment biopsies were preferred, but the most recent archived sample could be used. If no archived biopsies were available, a fresh biopsy was taken prior to dosing. Biopsies were retrospectively analyzed for membrane and cytoplasmic TF expression in a central laboratory using an analytically validated immunohistochemistry assay. TF histology-score (H-score) was calculated based on the percentage of tumor tissue that had membrane or cytoplasmic TF expression intensity of low (1+), intermediate (2+), and high (3+) on evaluable samples using the following equation: H-score=(1×[% cells 1+])+(2×[% cells 2+])+(3×[% cells 3+]).

Patient demographics and baseline characteristics are shown in Table 5. A total of 101 patients continued with therapy (99%) and 1 patient withdrew from treatment due to AE.

TABLE 5
Patient Demographics and Baseline Characteristics

| Baseline Demographics and Disease Characteristics | N = 101 | |
|---|---|---|
| Age (years) | | |
| Mean (SD) | 50.7 | (10.7) |
| Median (Range) | 50.0 | (31-78) |
| Race, n (%) | | |
| White | 96 | (95.0%) |
| Asian | 2 | (2.0%) |
| Black or African American | 1 | (1.0%) |
| Other | 2 | (2.0%) |
| ECOG performance status, n (%) | | |
| 0 | 59 | (58.4%) |
| 1 | 42 | (41.6%) |
| Histology, n (%) | | |
| Squamous cell carcinoma | 69 | (68.3%) |
| Adenocarcinoma | 27 | (26.7%) |
| Adenosquamous carcinoma | 5 | (5.0) |
| Recurrent disease, n (%) | | |
| Yes | 61 60.4%) | |
| No | 40 | (39.6%) |
| Prior radiation to pelvis, n (%) | | |
| Yes | 82 | (81.2%) |
| No | 19 | (18.8%) |
| Prior cisplatin + radiation | | |
| Yes | 55 | (54.5%) |
| No | 46 | (45.5%) |
| Prior lines of systemic therapies for | | |
| recurrent/metastatic disease, n (%) | | |
| 1 | 71 | (70.3%) |
| 2 | 30 | (29.7%) |
| Prior bevacizumab plus doublet chemotherapy* as | | |
| first-line therapy, n (%) | | |
| Yes | 64 | (63.4%) |
| No | 37 | (36.6%) |
| Response to last systemic regimen, n (%) | | |
| Yes | 38 | (37.6%) |
| No | 57 | (56.4%) |
| Unknown | 6 | (5.9%) |
| Time from last systemic regimen to start of | | |
| Tisotumab Vedotin (months) | | |
| n | 101 | |
| Mean (SD) | 6.1 | (6.8) |
| Median (Range) | 3.5 | (1; 48) |
| Biopsy evaluable, n (%) | 80 | (79) |
| Positive membrane TF expression**, n (%) | 77 | (96) |
| Extrapelvic metastatic disease at baseline, n (%) | 95 | (94) |

ECOG, Eastern Cooperative Oncology Group; TF, tissue factor.
*Doublet chemotherapy defined as paclitaxel-platinum or paclitaxel-topotecan.
**Positive TF expression defined as any positive membrane staining on tumor cells out of biopsy-evaluable population (n = 80).

### Results

The median treatment duration was 4.2 months (range, 1-16), the median number of doses of TV received was 6.0 (range, 1; 21), the median cumulative dose was 10.7 mg/kg (2; 33) and the median relative dose intensity was 95.9% (44; 114). 23 patients (22.8%) had an AE leading to 1 dose reduction and 1 patient (1.0%) had an AE leading to 2 dose reductions. The median follow-up time was 10.0 months (0.7; 17.9).

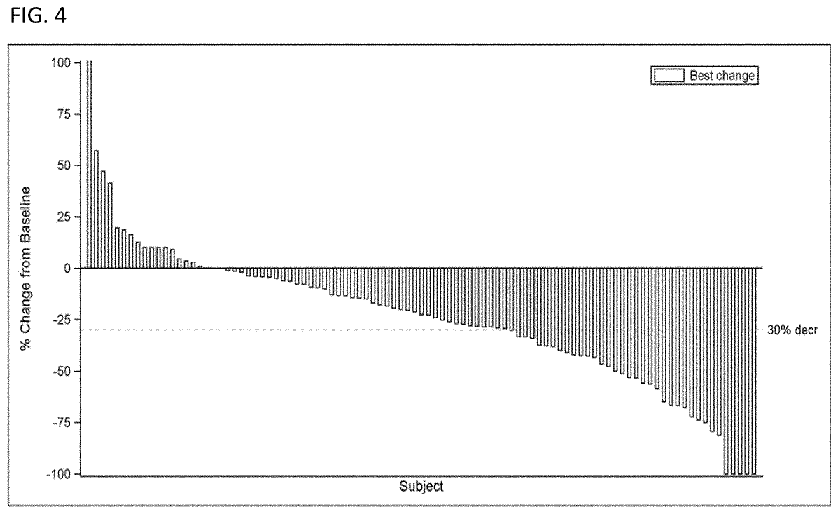

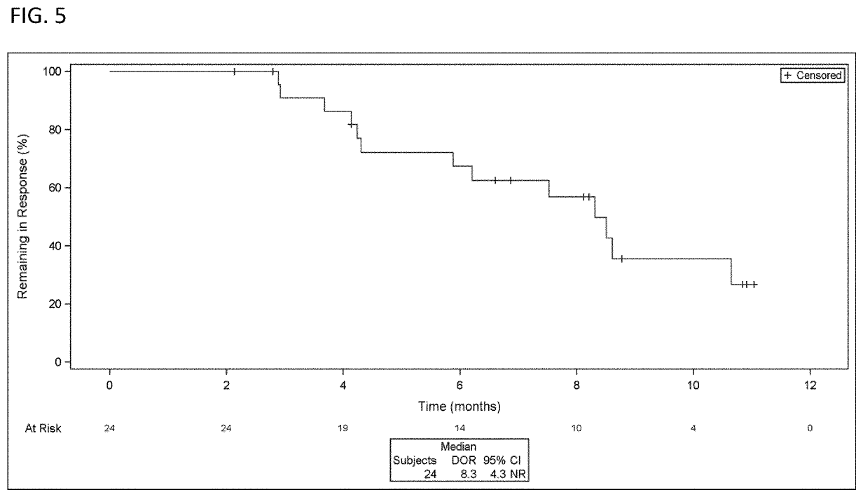

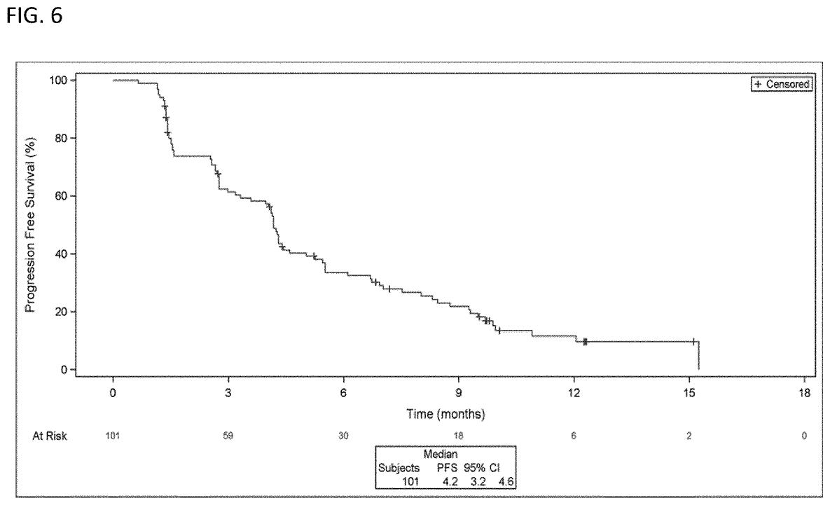

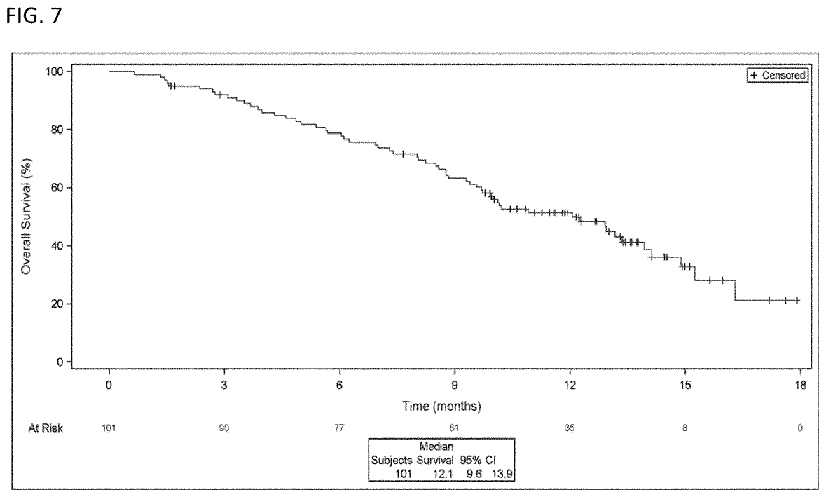

101 patients were evaluated for efficacy (Table 6 and FIG. 4 (target lesion response in each subject shown as the best percent change in lesion size relative to baseline for each subject), FIG. 5 (percent of subjects remaining in response over time), FIG. 6 (percent of subjects exhibiting progression-free survival over time), and FIG. 7 (percent of subjects surviving over time)). 4 patients continued to undergo treatment.

TABLE 6
Efficacy measurement

| Antitumor Activity Endpoints | N = 101 | |
|---|---|---|
| ORR* (95% CI), % | 23.8 | (15.9-33.3) |
| CR, n (%) | 7 | (6.9) |
| PR, n (%) | 17 | (16.8) |
| SD, n (%) | 49 | (48.5) |
| PD, n (%) | 24 | (23.8) |
| Not evaluable, n (%) | 4 | (4.0) |
| Median TTR* (range), months | 1.4 | (1.1-5.1) |
| Median DOR* (range), months | 8.3 | (2.1-11.0) |
| Median PFS* (95% CI), months | 4.2 | (3.2-4.6) |
| 3-month PFS rate (95% CI), % | 61.4 | (51.0-70.2) |
| 6-month PFS rate (95% CI), % | 33.6 | (24.3-13.1) |
| Median OS (95% CI), months | 12.1 | (9.6-13.9) |
| 6-month OS rate (95% CI), % | 78.7 | (69.3-85.6) |
| 12-month OS rate (95% CI), % | 51.4 | (40.9-60.8) |

CI, confidence interval; CR, complete response; DOR, duration of response; IRC, independent review committee; ORR, objective response rate; OS, overall survival; PD, progressive disease; PFS, progression-free survival; PR, partial response; SD, stable disease; TTR, time to response.
*IRC-assessed confirmed ORR, TTR, DOR, and PFS by Response Evaluation Criteria In Solid Tumors v1.1 criteria.
† Patients were not evaluable.

A total of 101 patients were evaluated for efficacy (Table 7).

TABLE 7
Efficacy measurement

| | | ORR^{a}, | | ORR 95% |
|---|---|---|---|---|
| | N | N (%)^{b} | | CI, % |
| All efficacy-evaluable patients | 101 | 24 | (23.8) | 15.9-33.3 |
| Histology | | | | |
| Non-squamos | 32 | 8 | (25) | 11.5-43.4 |
| Squamos | 69 | 16 | (23.2) | 13.9-34.9 |
| Prior cisplatin + radiation | | | | |
| Yes | 55 | 14 | (25.5) | 14.7-39.0 |
| No | 46 | 10 | (21.7) | 10.9-36.4 |
| Prior lines of systemic regimen | | | | |
| 1 | 71 | 20 | (28.2) | 18.1-40.1 |
| 2 | 30 | 4 | (13.3) | 3.8-30.7 |
| Rse to last systemic | | | | |
| regimen^{c} | | | | |
| Yes | 38 | 10 | (26.3) | 13.4-43.1 |
| No | 57 | 12 | (21.1) | 11.4-33.9 |
| Prior bevacizumab in | | | | |
| combination with chemotherapy | | | | |
| as doublet 1 L therapy^{d} | | | | |
| Yes | 64 | 12 | (18.8) | 10.1-30.5 |
| No | 37 | 12 | (32.4) | 18.0-49.8 |
| ECOG | | | | |
| 0 | 59 | 18 | (30.5) | 19.2-43.9 |
| 1 | 42 | 6 | (14.3) | 5.4-28.5 |
| Age | | | | |
| <65 years | 88 | 24 | (27.3) | 18.3-37.8 |
| ≥65 years | 13 | 0 | (0.0) | 0.0-24.7 |

^{a}Indicates Objective Response Rate.
^{b}combined uncomfirmed + confirmed ORR.
^{c}response to last systemic regimen was not available for 6 subjects.
^{d}chemotherapy doublet includes either: paclitaxel + cisplatin/carboplatin or paclitaxel + topotecan.

### Statistical Analyses

Study size was calculated assuming a confirmed ORR of 21%-25% with TV and to provide ≥80% power to exclude an ORR of ≤11%. All patients who received at least one dose of TV were included in the efficacy and safety analyses. ORR was tested using an exact test at a one-sided 2.5% alpha level. An exact 95% two-sided confidence interval (CI) for the ORR was calculated using the Clopper-Pearson method. Patients with missing response data were counted as non-responders. Median DOR, PFS, and OS were estimated using the Kaplan-Meier method and presented with a two-sided 95% CI. Prespecified subgroup analyses were performed, including histology, number of prior lines of therapy, prior radiation therapy for localized disease, prior bevacizumab, response to last therapy, and TF expression by immunohistochemistry.

### CONCLUSION

R/mCC is a devastating disease with a poor prognosis and no current 2L+ SOC therapy. The high risk of relapse after 1L therapy and low ORR and survival with existing treatment options in the 2L+ setting highlight the need for new, safe, and effective treatments that improve clinical benefit in this patient population. TV demonstrated a manageable safety profile and a significant ORR with durable responses, thus providing overall clinical benefit in patients with limited treatment options. To our knowledge, TV is the first and only antibody-drug conjugate targeting TF to successfully demonstrate meaningful clinical activity. The safety profile of TV was manageable and generally consistent with other MMAE-based antibody-drug conjugates. See e.g., Prince et al., 2017, Lancet, 390:555-56 and Bendell et al., 2014, Official Journal of the Am. Soc. of Clin. Oncol., 32: 3619-25.

101 patients were treated with TV. ORR was 23.8% (24/101 patients; 95% confidence interval [CI], 15.9%-33.3%; p=0.0002), with 7 patients having a complete response. Median duration of follow-up was 10.0 months (range, 0.7-17.9). Median DOR was 8.3 months (range, 2.1-11.0). Median PFS was 4.2 months (95% CI, 3.2-4.6), with a 6-month PFS rate of 33.6%. Median OS was 12.1 months (95% CI, 9.6-13.9), with a 12-month OS rate of 51.4%. One treatment-related death occurred.

IRC-assessed ORR was observed across histologic types (squamous cell carcinoma, 23.2% [16/69 patients]; non-squamous, 25.0% [8/32]); number of prior lines of therapy (one prior line, 28.2% [20/71]); two prior lines, 13.3% [4/3]); prior cisplatin+radiation (yes, 25.5% [14/55]; no, 21.7% [10/46]); prior bevacizumab in combination with chemotherapy doublet (yes, 18.8% [12/64]; no, 32.4 [12/37]); ECOG performance status (0, 30.5% [18/59]; 1, 14.3% (6/42); and age (less than 65 years old, 27.3% [24/88]; greater than or equal to 65 years old, 0% [0/13]).

Treatment with TV in patients with r/mCC demonstrated a significant ORR in the 2L/3L setting:
Responses with TV were observed across subgroups of interest, including histology, line of therapy, and prior doublet chemotherapy plus bevacizumab.
Responses were durable with some patients having a response for >11 months, including some patients who discontinued treatment.
Prolonged responses with TV led to meaningful PFS and OS benefit for patients.
The durability of response with TV may be indicative of its multiple mechanisms of action, including MMAE-induced direct cytotoxicity, bystander killing, and ICD, as well as Fcγ receptor-mediated effector functions and inhibition of TF/FVIIa signaling (see de Goeij et al., 2015 and Mol. Cancer. Ther., 14:1130-40, Breij et al., 2014, Cancer Res., 74:1214-26).
Considering the poor response rates observed with other treatment options (<15%) and limited addressable patient populations for some therapies (see e.g., Miller et al., 2008, Gynecol. Oncol. 110:65-70; Monk et al., 2009, J. Clin. Oncol. 27:1069-1074; Muggia et al., 2004, Gynecol. Oncol. 92:639-643; Garcia et. al., 2007, Am. J. Clin. Oncol. 30-428-431; Schilder et al., 2005, Gynecol. Oncol. 96:103-107; and Corp. MSD. KEYTRUDA® (pembrolizumab) for injection, for intravenous use. Whitehouse Station, NJ: Merck & Co., Inc.; 06/2018), the broad antitumor activity observed with TV supports its continued clinical development in patients with previously treated r/mCC.

Overall, the results of this study demonstrated that TV provides clinical benefit to patients with r/mCC. The benefit: risk profile of tisotumab vedotin is considered favorable in patients with recurrent or metastatic cervical cancer who have received at least one prior systemic therapy, which is a patient population with high unmet medical need.

What is claimed is:

1. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject has been previously treated with bevacizumab.

2. The method of claim 1, wherein the subject has an ECOG score of 0.

3. The method of claim 1, wherein the subject has an ECOG score of 1.

4. The method of any one of claims 1-3, wherein the subject is less than 65 years old.

5. The method of any one of claims 1-4, wherein the subject has been previously treated with:
a) paclitaxel and cisplatin,
b) paclitaxel and carboplatin, or
c) paclitaxel and topotecan.

6. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject has not been previously treated with bevacizumab.

7. The method of claim 6, wherein the subject has an ECOG score of 0.

8. The method of claim 6, wherein the subject has an ECOG score of 1.

9. The method of any one of claims 6-8, wherein the subject is less than 65 years old.

10. The method of any one of claims 6-9, wherein the subject has been previously treated with:
a) paclitaxel and cisplatin,
b) paclitaxel and carboplatin, or
c) paclitaxel and topotecan.

11. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject has an Eastern Cooperative Oncology Group (ECOG) score of 0.

12. The method of claim 11, wherein the subject is less than 65 years old.

13. The method of any claim 11 or 12, wherein the subject has been previously treated with:
a) paclitaxel and cisplatin,
b) paclitaxel and carboplatin, or
c) paclitaxel and topotecan.

14. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject has an ECOG score of 1.

15. The method of claim 14, wherein the subject is less than 65 years old.

16. The method of any claim 14 or 15, wherein the subject has been previously treated with:
a) paclitaxel and cisplatin,
b) paclitaxel and carboplatin, or
c) paclitaxel and topotecan.

17. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject is less than 65 years old.

18. The method of claim 17, wherein the subject has an ECOG score of 0.

19. The method of claim 17, wherein the subject has an ECOG score of 1.

20. The method of any one of claims 17-19, wherein the subject has been previously treated with:
a) paclitaxel and cisplatin,
b) paclitaxel and carboplatin, or
c) paclitaxel and topotecan.

21. The method of any one of claims 1-20, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.

22. The method of any one of claims 1-21, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.

23. The method of any one of claims 1-22, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.

24. The method of any one of claims 1-23, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.

25. The method of any one of claims 1-24, wherein the time to response is less than about 6 months after administration of the antibody-drug conjugate, optionally, wherein the time to response is less than about 4 months, about 2 months, 1.4 months, or about 1.2 months after administration of the antibody-drug conjugate.

26. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.

27. The method of claim 26, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.

28. The method of claim 26 or 27, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.

29. The method of any one of claims 26-28, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.

30. The method of any one of claims 26-29, wherein the time to response is less than about 6 months after administration of the antibody-drug conjugate, optionally, wherein the time to response is less than about 4 months, about 2 months, 1.4 months, or about 1.2 months after administration of the antibody-drug conjugate.

31. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.

32. The method of claim 31, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.

33. The method of claim 31 or 32, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.

34. The method of any one of claims 31-33, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.

35. The method of any one of claims 31-34, wherein the time to response is less than about 6 months after administration of the antibody-drug conjugate, optionally, wherein the time to response is less than about 4 months, about 2 months, 1.4 months, or about 1.2 months after administration of the antibody-drug conjugate.

36. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.

37. The method of claim 36, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.

38. The method of claim 36 or 37, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.

39. The method of any one of claims 36-38, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.

40. The method of any one of claims 36-39, wherein the time to response is less than about 6 months after administration of the antibody-drug conjugate, optionally, wherein the time to response is less than about 4 months, about 2 months, 1.4 months, or about 1.2 months after administration of the antibody-drug conjugate.

41. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.

42. The method of claim 41, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.

43. The method of claim 41 or 42, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.

44. The method of any one of claims 41-43, wherein the objective response rate is between about 13% and about 35%, optionally, wherein the objective response rate is at least about 14%, about 19%, about 21%, 23.8%, about 24%, about 25%, about 26%, about 28%, about 30%, or about 33%.

45. The method of any one of claims 41-44, wherein the time to response is less than about 6 months after administration of the antibody-drug conjugate, optionally, wherein the time to response is less than about 4 months, about 2 months, 1.4 months, or about 1.2 months after administration of the antibody-drug conjugate.

46. A method of treating cervical cancer in a subject, the method comprising administering to the subject an antibody-drug conjugate that binds to tissue factor (TF), wherein the antibody-drug conjugate is tisotumab vedotin or a biosimilar thereof, and wherein the antibody-drug conjugate is administered at a dose of 2.0 mg/kg once every 3 weeks, wherein the time to response is less than about 6 months after administration of the antibody-drug conjugate, optionally, wherein the time to response is less than about 4 months, about 2 months, 1.4 months, or about 1.2 months after administration of the antibody-drug conjugate.

47. The method of claim 46, wherein the subject exhibits progression-free survival of at least about 3 months after administration of the antibody-drug conjugate, optionally, wherein the subject exhibits progression-free survival of at least about 4 months, about 5 months, or about 6 months after administration of the antibody-drug conjugate.

48. The method of claim 46 or 47, wherein the subject exhibits overall survival of at least about 10 months after administration of the antibody-drug conjugate, optionally, wherein the subject exhibits overall survival of at least about 11 months, about 12 months, about 13 months, or 14 months after administration of the antibody-drug conjugate.

49. The method of any one of claims 46-48, wherein the duration of response to the antibody-drug conjugate is at least about 6 months after administration of the antibody-drug conjugate, optionally, wherein the duration of response to the antibody-drug conjugate is at least about 7 months, about 8 months or about 10 months after administration of the antibody-drug conjugate.

50. The method of any one of claims 46-49, wherein the objective response rate is at least about between about 13% and about 35%, optionally, wherein the objective response rate is at least about 14%, about 19%, about 21%, 23.8%, about 24%, about 25%, about 26%, about 28%, about 30%, or about 33%.

51. The method of any one of claims 26-50, wherein the subject has been previously treated with bevacizumab.

52. The method of any one of claims 26-50, wherein the subject has not been previously treated with bevacizumab.

53. The method of any one of claims 26-52, wherein the subject has experienced disease progression during or after treatment with:
a) paclitaxel and cisplatin,
b) paclitaxel and carboplatin,
c) paclitaxel and topotecan,
d) bevacizumab, paclitaxel, and cisplatin,
e) bevacizumab, paclitaxel, and carboplatin, or
f) bevacizumab, paclitaxel, and topotecan.

54. The method of any one of claims 26-53, wherein the subject is less than 65 years old.

55. The method of any one of claims 26-54, wherein the subject has an ECOG score of 0.

56. The method of any one of claims 26-54, wherein the subject has an ECOG score of 1.

57. The method of any one of claims 1-56, wherein the cervical cancer is an adenocarcinoma.

58. The method of any one of claims 1-56, wherein the cervical cancer is an adenosquamous carcinoma.

59. The method of any one of claims 1-56, wherein the cervical cancer is a squamous cell carcinoma.

60. The method of any one of claims 1-56, wherein the cervical cancer is a non-squamous cell carcinoma.

61. The method of any one of claims 1-60, wherein the cervical cancer is recurrent or metastatic cervical cancer.

62. The method of any one of claims 1-61, wherein the subject has been previously treated with one or more therapeutic agents and did not respond to the treatment, wherein the one or more therapeutic agents is not the antibody-drug conjugate.

63. The method of any one of claims 1-61, wherein the subject has been previously treated with one or more therapeutic agents and relapsed after the treatment, wherein the one or more therapeutic agents is not the antibody-drug conjugate.

64. The method of any one of claims 1-61, wherein the subject has been previously treated with one or more therapeutic agents and has experienced disease progression during treatment, wherein the one or more therapeutic agents is not the antibody-drug conjugate.

65. The method of any one of claims 62-64, wherein the one or more therapeutic agents is a platinum-based therapeutic agent.

66. The method of any one of claims 62-64, wherein the one or more therapeutic agents is selected from the group consisting of: paclitaxel, cisplatin, carboplatin, topotecan, gemcitabine, fluorouracil, ixabepilone, imatinib mesylate, docetaxel, gefitinib, nab-paclitaxel, pemetrexed, vinorelbine, doxil, cetuximab, pembrolizumab, nivolumab and bevacizumab.

67. The method of any one of claims 1-66, wherein the subject is not a candidate for curative therapy.

68. The method of claim 67, wherein the curative therapy comprises radiotherapy and/or exenterative surgery.

69. The method of any one of claims 1-66, wherein the subject has received prior radiation to the pelvis.

70. The method of any one of claims 1-66, wherein the subject has not received prior radiation to the pelvis.

71. The method of any one of claims 1-70, wherein the subject has received one prior line of systemic therapy for relapsed, recurrent or metastatic cancer.

72. The method of any one of claims 1-70, wherein the subject has received two prior lines of systemic therapy for relapsed, recurrent or metastatic cancer.

73. The method of any one of claims 71 or 72, wherein the subject did not respond to treatment with the prior systemic therapy.

74. The method of any one of claims 71 or 72, wherein the subject relapsed after treatment with the prior systemic therapy.

75. The method of any one of claims 1-74, wherein the cervical cancer is an advanced stage cervical cancer, such as a stage 3 or stage 4 cervical cancer, such as metastatic cervical cancer.

76. The method of any one of claims 1-75, wherein the cervical cancer is recurrent cervical cancer.

77. The method of any one of claims 1-76, wherein the route of administration for the antibody-drug conjugate is intravenous.

78. The method of any one of claims 1-77, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF.

79. The method of any one of claims 1-78, wherein the subject has a TF histology score (H-score) of at least 1.

80. The method of any one of claims 1-79, wherein the subject has one or more adverse events and is further administered an additional therapeutic agent to eliminate or reduce the severity of the one or more adverse events.

81. The method of any one of claims 1-79, wherein the subject is at risk of developing one or more adverse events and is further administered an additional therapeutic agent to prevent or reduce the severity of the one or more adverse events.

82. The method of any one of claims 1-80, wherein the subject has one or more adverse events and the dose of the antibody drug conjugate is reduced following the one or more adverse events.

83. The method of claim 82, wherein the dose is reduced from 2.0 mg/kg to 1.3 mg/kg.

84. The method of claim 83, wherein the dose is reduced from 1.3 mg/kg to 0.9 mg/kg.

85. The method of any one of claims 80-84, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.

86. The method of any one of claims 80-85, wherein the one or more adverse events is a grade 3 or greater adverse event.

87. The method of any one of claims 80-85, wherein the one or more adverse events is a serious adverse event.

88. The method of claim 80 or claim 81, wherein the one or more adverse events is conjunctivitis and/or keratitis and the additional agent is a preservative-free lubricating eye drop, an ocular vasoconstrictor and/or a steroid eye drop.

89. The method of any one of claims 1-88, wherein the antibody-drug conjugate is administered as a monotherapy.

90. The method of any one of claims 1-89, wherein the subject is a human.

91. The method of any one of claims 1-90, wherein the antibody-drug conjugate is in a pharmaceutical composition comprising the antibody-drug conjugate and a pharmaceutical acceptable carrier.

92. An antibody-drug conjugate that binds to TF for use in the method of any one of claims 1-91.

93. Use of an antibody-drug conjugate that binds to TF for the manufacture of a medicament for use in the method of any one of claims 1-91.
